SEQUENCE LISTING (57 sequences)

SEQ ID NO: 1 - DNA, 745 bp, synthetic construct
  source 1..745: mol_type = other DNA
atggcccaggtgaagctgcagcagtcaggacctgacctggtgaagcctggggcttcagtgaagatatcctgcaaggcgtctggttactcattcactggctactacatgcactgggtgaagcagagccatggaaagagccttgagtggattggacgtgttaatcctaacagtggtggtacaagctacaaccagaagttcaaggacaaggccatattaactgtagacaagtcatccagcacagcctacatggagctccgcagcctgacatctgaggactctgcggtctattactgtgcaagatcgaaaggtaactacttctatgctatggactactggggccaagggaccacggtcaccgtctcctcaagtggaggcggttcaggcggaggtggctctggcggtggcggatcggacatcgagctcactcagtctccagcttctttggctgtgtctctagggcagagggccaccatatcctgcagagccagtgaaagtgttgatagtcatggcactagttttatgcactggtaccagcagaaaccaggacagccacccaaattcctcatctatcgtgcatccaacctagaatctgggatccctgccaggttcagtggcagtgggtctaggacagacttcaccctcaccattaatcctgtggagacagatgatgttgcaatctattactgtcagcaaagtaatgaggatcctccgacgttcggtggaggcaccaagctggaaatcaaac

SEQ ID NO: 2 - DNA, 361 bp, synthetic construct
  source 1..361: mol_type = other DNA
caggtgaagctgcagcagtcaggacctgacctggtgaagcctggggcttcagtgaagatatcctgcaaggcgtctggttactcattcactggctactacatgcactgggtgaagcagagccatggaaagagccttgagtggattggacgtgttaatcctaacagtggtggtacaagctacaaccagaagttcaaggacaaggccatattaactgtagacaagtcatccagcacagcctacatggagctccgcagcctgacatctgaggactctgcggtctattactgtgcaagatcgaaaggtaactacttctatgctatggactactggggccaagggaccacggtcaccgtctcctcaa

SEQ ID NO: 3 - protein, 120 aa, synthetic construct
QVKLQQSGPDLVKPGASVKISCKASGYSFTGYYMHWVKQSHGKSLEWIGRVNPNSGGTSYNQKFKDKAILTVDKSSSTAYMELRSLTSEDSAVYYCARSKGNYFYAMDYWGQGTTVTVSS

SEQ ID NO: 4 - DNA, 334 bp, synthetic construct
  source 1..334: mol_type = other DNA
gacatcgagctcactcagtctccagcttctttggctgtgtctctagggcagagggccaccatatcctgcagagccagtgaaagtgttgatagtcatggcactagttttatgcactggtaccagcagaaaccaggacagccacccaaattcctcatctatcgtgcatccaacctagaatctgggatccctgccaggttcagtggcagtgggtctaggacagacttcaccctcaccattaatcctgtggagacagatgatgttgcaatctattactgtcagcaaagtaatgaggatcctccgacgttcggtggaggcaccaagctggaaatcaaac

SEQ ID NO: 5 - protein, 111 aa, synthetic construct
DIELTQSPASLAVSLGQRATISCRASESVDSHGTSFMHWYQQKPGQPPKFLIYRASNLESGIPARFSGSGSRTDFTLTINPVETDDVAIYYCQQSNEDPPTFGGGTKLEIK

SEQ ID NO: 6 - protein, 120 aa, synthetic construct
EVQLVESGGGLVQPGGSLRLSCKASGYSFTGYYMHWVRQAPGKGLEWIGRVNPNSGGTSYNQKFKDRATLSVDNSKNTAYLQMNSLRAEDTAVYYCARSKGNYFYAMDYWGQGTLVTVSS

SEQ ID NO: 7 - protein, 120 aa, synthetic construct
EVQLVESGGGLVQPGGSLKVSCKASGYSFTGYYMHWVRQASGKGLEWIGRVNPNSGGTSYNQKFKDRFTISRDKSISTLYLQMSSLRSEDTAVYYCARSKGNYFYAMDYWGQGTLVTVSS

SEQ ID NO: 8 - protein, 120 aa, synthetic construct; identical to SEQ ID NO: 3

SEQ ID NO: 9 - protein, 111 aa, synthetic construct
DIQMTQSPSSLSASVGDRVTITCRASESVDSHGTSFMHWYQQKPGKAPKFLIYRASNLESGIPSRFSGSGSGTDFTLTISSVQPEDFAVYYCQQSNEDPPTFGGGTKVEIK

SEQ ID NO: 10 - protein, 111 aa, synthetic construct
DIQMTQSPSSLSASVGDRVTISCRASESVDSHGTSFMHWYQQKPGKSPKFLIYRASNLESGIPSRFSGSGSGTDFTLTISSLQPEDFAIYYCQQSNEDPPTFGGGTKVEIK

SEQ ID NO: 11 - protein, 111 aa, synthetic construct; identical to SEQ ID NO: 5

SEQ ID NO: 12 - protein, 246 aa, synthetic construct
EVQLVESGGGLVQPGGSLRLSCKASGYSFTGYYMHWVRQAPGKGLEWIGRVNPNSGGTSYNQKFKDRATLSVDNSKNTAYLQMNSLRAEDTAVYYCARSKGNYFYAMDYWGQGTLVTVSSGGGGSGGGGSGGGGSDIQMTQSPSSLSASVGDRVTITCRASESVDSHGTSFMHWYQQKPGKAPKFLIYRASNLESGIPSRFSGSGSGTDFTLTISSVQPEDFAVYYCQQSNEDPPTFGGGTKVEIK

SEQ ID NO: 13 - protein, 246 aa, synthetic construct
EVQLVESGGGLVQPGGSLKVSCKASGYSFTGYYMHWVRQASGKGLEWIGRVNPNSGGTSYNQKFKDRFTISRDKSISTLYLQMSSLRSEDTAVYYCARSKGNYFYAMDYWGQGTLVTVSSGGGGSGGGGSGGGGSDIQMTQSPSSLSASVGDRVTISCRASESVDSHGTSFMHWYQQKPGKSPKFLIYRASNLESGIPSRFSGSGSGTDFTLTISSLQPEDFAIYYCQQSNEDPPTFGGGTKVEIK

SEQ ID NO: 14 - protein, 17 aa, synthetic construct
SSGGGGSGGGGSGGGGS

SEQ ID NO: 15 - protein, 246 aa, synthetic construct; identical to SEQ ID NO: 12

SEQ ID NO: 16 - DNA, 738 bp, synthetic construct
  source 1..738: mol_type = other DNA
gaggtgcagctggtggagagcggcggcggcctggtgcagcccggcggcagcctgaggctgagctgcaaggccagcggctacagcttcaccggctactacatgcactgggtgaggcaggcccccggcaagggcctggagtggatcggcagggtgaaccccaacagcggcggcaccagctacaaccagaagttcaaggacagggccaccctgagcgtggacaacagcaagaacaccgcctacctgcagatgaacagcctgagggccgaggacaccgccgtgtactactgcgccaggagcaagggcaactacttctacgccatggactactggggccagggcaccctggtgaccgtgagcagcggcggcggcggcagcggcggcggcggcagcggcggcggcggcagcgacatccagatgacccagagccccagcagcctgagcgccagcgtgggcgacagggtgaccatcacctgcagggccagcgagagcgtggacagccacggcaccagcttcatgcactggtaccagcagaagcccggcaaggcccccaagttcctgatctacagggccagcaacctggagagcggcatccccagcaggttcagcggcagcggcagcggcaccgacttcaccctgaccatcagcagcgtgcagcccgaggacttcgccgtgtactactgccagcagagcaacgaggacccccccaccttcggcggcggcaccaaggtggagatcaag

SEQ ID NO: 17 - protein, 15 aa, synthetic construct
SLDKHKHKKLQSFYP

SEQ ID NO: 18 - protein, 246 aa, synthetic construct; identical to SEQ ID NO: 13

SEQ ID NO: 19 - DNA, 738 bp, synthetic construct
  source 1..738: mol_type = other DNA
gaggtgcagctggtggagagcggcggcggcctggtgcagcccggcggcagcctgaaggtgagctgcaaggccagcggctacagcttcaccggctactacatgcactgggtgaggcaggccagcggcaagggcctggagtggatcggcagggtgaaccccaacagcggcggcaccagctacaaccagaagttcaaggacaggttcaccatcagcagggacaagagcatcagcaccctgtacctgcagatgagcagcctgaggagcgaggacaccgccgtgtactactgcgccaggagcaagggcaactacttctacgccatggactactggggccagggcaccctggtgaccgtgagcagcggcggcggcggcagcggcggcggcggcagcggcggcggcggcagcgacatccagatgacccagagccccagcagcctgagcgccagcgtgggcgacagggtgaccatcagctgcagggccagcgagagcgtggacagccacggcaccagcttcatgcactggtaccagcagaagcccggcaagagccccaagttcctgatctacagggccagcaacctggagagcggcatccccagcaggttcagcggcagcggcagcggcaccgacttcaccctgaccatcagcagcctgcagcccgaggacttcgccatctactactgccagcagagcaacgaggacccccccaccttcggcggcggcaccaaggtggagatcaag

SEQ ID NO: 20 - DNA, 336 bp, synthetic construct
  source 1..336: mol_type = other DNA
tttgaggtccagctgcagcagtctggggcagagcttgtgaagccaggggcctcagtcaagttgtcctgcacagcttctggcttcaacattaaagacacctatatacacttcgtgaggcagaggcctgaacagggcctggagtggattggaaggattgatcctgcgaatgataatactttatatgcctcaaagttccagggcaaggccactataacagcagacacatcatccaacacagcctacatgcacctctgcagcctgacatctggggacactgccgtctattactgtggtagaggttatggttactacgtatttgaccacttgggccaaggc

SEQ ID NO: 21 - protein, 112 aa, synthetic construct
FEVQLQQSGAELVKPGASVKLSCTASGFNIKDTYIHFVRQRPEQGLEWIGRIDPANDNTLYASKFQGKATITADTSSNTAYMHLCSLTSGDTAVYYCGRGYGYYVFDHLGQG

SEQ ID NO: 22 - DNA, 348 bp, synthetic construct
  source 1..348: mol_type = other DNA
tntcaggtgcagctgaagcagtctggggcagagcttgtgaagccaggggcctcagtcaagttgtcctgcacagcttctggcttcaacattaaagacacctatatacacttcgtgaggcagaggcctgaacagggcctggagtggattggaaggattgatcctgcgaatgataatactttatatgcctcaaagttccagggcaaggccactataacagcagacacatcatccaacacagcctacatgcacctctgcagcctgacatctggggacactgccgtctattactgtggtagaggttatggttactacgtatttgaccactggggccaaggcaccactctcaca

SEQ ID NO: 23 - protein, 116 aa, synthetic construct
XQVQLKQSGAELVKPGASVKLSCTASGFNIKDTYIHFVRQRPEQGLEWIGRIDPANDNTLYASKFQGKATITADTSSNTAYMHLCSLTSGDTAVYYCGRGYGYYVFDHWGQGTTLT

SEQ ID NO: 24 - DNA, 348 bp, synthetic construct
  source 1..348: mol_type = other DNA
tttgaggtccagctgcagcagtctggggcagagcttgtgaagccaggggcctcagtcaagttgtcctgcacagcttctggcttcaacattaaagacacctatatacacttcgtgaggcagaggcctgaacagggcctggagtggattggaaggattgatcctgcgaatgataatactttatatgcctcaaagttccagggcaaggccactataacagcagacacatcatccaacacagcctacatgcacctctgcagcctgacatctggggacactgccgtctattactgtggtagaggttatggttactacgtatttgaccactggggccaaggcaccactctcaca

SEQ ID NO: 25 - protein, 116 aa, synthetic construct
FEVQLQQSGAELVKPGASVKLSCTASGFNIKDTYIHFVRQRPEQGLEWIGRIDPANDNTLYASKFQGKATITADTSSNTAYMHLCSLTSGDTAVYYCGRGYGYYVFDHWGQGTTLT

SEQ ID NO: 26 - DNA, 347 bp, synthetic construct
  source 1..347: mol_type = other DNA
tcaggtccaactgcagcagcctggggcagagcttgtgaagccaggggcctcagtcaagttgtcctgcacagcttctggcttcaacattaaagacacctatatacacttcgtgaggcagaggcctgaacagggcctggagtggattggaaggattgatcctgcgaatgataatactttatatgcctcaaagttccagggcaaggccactataacagcagacacatcatccaacacagcctacatgcacctctgcagcctgacatctggggacactgccgtctattactgtggtagaggttatggttactacgtatttgaccactggggccaaggcaccactctcacat

SEQ ID NO: 27 - protein, 115 aa, synthetic construct
QVQLQQPGAELVKPGASVKLSCTASGFNIKDTYIHFVRQRPEQGLEWIGRIDPANDNTLYASKFQGKATITADTSSNTAYMHLCSLTSGDTAVYYCGRGYGYYVFDHWGQGTTLT

SEQ ID NO: 28 - DNA, 317 bp, synthetic construct
  source 1..317: mol_type = other DNA
gacattgtgctgacccaatctcctgcttccttagctgtatctctggggcagagggccaccatctcatacagggccagcaaaagtgtcagtacatctggctatagttatatgcactggaaccaacagaaaccaggacagccacccagactcctcatctatcttgtatccaacctagaatctggggtccctgccaggttcagtggcagtgggtctgggacagacttcaccctcaacatccatcctgtggaggaggaggatgctgcaacctattactgtcagcacattagggagcttacacgttcggagggggaccaagc

SEQ ID NO: 29 - protein, 105 aa, synthetic construct
DIVLTQSPASLAVSLGQRATISYRASKSVSTSGYSYMHWNQQKPGQPPRLLIYLVSNLESGVPARFSGSGSGTDFTLNIHPVEEEDAATYYCQHIRELTRSEGDQ

SEQ ID NO: 30 - DNA, 299 bp, synthetic construct
  source 1..299: mol_type = other DNA
gatattgtgctaactcagtctcctgcttccttagctgtatctctggggcagagggccaccatctcatacagggccagcaaaagtgtcagtacatctggctatagttatatgcactggaaccaacagaaaccaggacagccacccagactcctcatctatcttgtatccaacctagaatctggggtccctgccaggttcagtggcagtgggtctgggacagacttcaccctcaacatccatcctgtggaggaggaggatgctgcaacctattactgtcagcacattagggagcttacacg

SEQ ID NO: 31 - protein, 99 aa, synthetic construct
DIVLTQSPASLAVSLGQRATISYRASKSVSTSGYSYMHWNQQKPGQPPRLLIYLVSNLESGVPARFSGSGSGTDFTLNIHPVEEEDAATYYCQHIRELT

SEQ ID NO: 32 - DNA, 307 bp, synthetic construct
  source 1..307: mol_type = other DNA
gacatccagctgactcagtctcctgcttccttagctgtatctctggggcagagggccaccatctcatacagggccagcaaaagtgtcagtacatctggctatagttatatgcactggaaccaacagaaaccaggacagccacccagactcctcatctatcttgtatccaacctagaatctggggtccctgccaggttcagtggcagtgggtctgggacagacttcaccctcaacatccatcctgtggaggaggaggatgctgctgacctattagctgtcagcacattatgggagcttacacgttcgg

SEQ ID NO: 33 - protein, 102 aa, synthetic construct
DIQLTQSPASLAVSLGQRATISYRASKSVSTSGYSYMHWNQQKPGQPPRLLIYLVSNLESGVPARFSGSGSGTDFTLNIHPVEEEDAADLLAVSTLWELTRS

SEQ ID NO: 34 - DNA, 1542 bp, synthetic construct
  source 1..1542: mol_type = other DNA
accggtatggatatcgagctgacccagagccctagcagcctggccgtgtcactgggccagagagccaccatcagctgcagagcctccgagagcgtggatagccacggcaccagcctgatgcactggtatcagcagaagcccggccagccccccaagttcctgatctaccgggccagcaacctggaaagcggcatccccgccagattttccggcagcggcagcagaaccgacttcaccctgaccatcaaccccgtggagacagacgacgtggccatctactactgccagcagagcaacgaggaccctcccacctttggcggaggcaccaagctggaactgaaggagggcggaggaagcggagggggaggatctggcggaggcggcagcgcccaggtgaagctgcagcagagcggccctgatctggtgaagcctggcgccagcgtgaagatcagctgcaaggccagcggctacagcttcaccggctactacatgcactgggtgaaacagagccacggcaagagcctggaatggatcggcagagtgaaccccaatagcggcggcaccagctacaaccagaagttcaaggacaaggccatcctgaccgtggacaagagcagcagcaccgcctacatggaactgcggagcctgaccagcgaggacagcgccgtgtactactgcgcccggtccaagggcaactacttctacgccatggactactggggccagggcaccaccgtgaccgtgtctagcagcggcggcggcggaagcgaagtgcagctgcagcagtccggagcagaactggtgaaacccggagccagtgtgaagctgagctgtacagcctccggctttaatatcaaggacacctacatccacttcgtgcgccaacggccagaacagggtctggagtggattggcaggatcgatccagcaaatgataacaccctgtacgcaagcaaatttcagggcaaagccacgataaccgccgatacatctagtaatacggcttacatgcacctctgctccctgacttccggggacaccgccgtgtattattgcgggcgcggatacggttacgtgttcgatcattggggtcagggcaccctcacagtctccagtgcaggaggcggaggtagtggaggcggaggctctggcggtggcgggtcagacatagtgctgacacagtctcccgccagtctggcagtgagccttggccagagggctacaatttcctatagggcgtctaaatccgttagcacttcaggttactcttatatgcactggaaccagaagcctggtcagcccaggcttcttatttacctggtcagcaatctcgagtctggcgtgcccgctagattttccggcagcgggagtgggactgacttcactctgaacatccacccagtggaggaagaggatgccgcaacctactattgtcagcatatccgggaattgaccagatcagagggcggaccttcctggaaggagcagaagctgattagcgaggaagatctggattataaggacgatgacaaatgagaattc

SEQ ID NO: 35 - protein, 518 aa, synthetic construct
TGMDIELTQSPSSLAVSLGQRATISCRASESVDSHGTSLMHWYQQKPGQPPKFLIYRASNLESGIPARFSGSGSRTDFTLTINPVETDDVAIYYCQQSNEDPPTFGGGTKLELKEGGGSGGGGSGGGGSAQVKLQQSGPDLVKPGASVKISCKASGYSFTGYYMHWVKQSHGKSLEWIGRVNPNSGGTSYNQKFKDKAILTVDKSSSTAYMELRSLTSEDSAVYYCARSKGNYFYAMDYWGQGTTVTVSSSGGGGSEVQLQQSGAELVKPGASVKLSCTASGFNIKDTYIHFVRQRPEQGLEWIGRIDPANDNTLYASKFQGKATITADTSSNTAYMHLCSLTSGDTAVYYCGRGYGYYVFDHWGQGTTLTVSSAGGGGSGGGGSGGGGSDIVLTQSPASLAVSLGQRATISYRASKSVSTSGYSYMHWNQQKPGQPPRLLIYLVSNLESGVPARFSGSGSGTDFTLNIHPVEEEDAATYYCQHIRELTRSEGGPSWKEQKLISEEDLDYKDDDDKEF

SEQ ID NO: 36 - DNA, 1560 bp, synthetic construct
  source 1..1560: mol_type = other DNA
accggtatggatatcgagctgacccagagccctagcctggccgtgtcactgggccagagagccaccatcagctgcagagcctccgagagcgtggatagccacggcaccagcctgatgcactggtatcagcagaagcccggccagccccccaagttcctgatctaccgggccagcaacctggaaagcggcatccccgccagattttccggcagcggcagcagaaccgacttcaccctgaccatcaaccccgtggagacagacgacgtggccatctactactgccagcagagcaacgaggaccctcccacctttggcggaggcaccaagctggaactgaaggagggcggaggaagcggagggggaggatctggcggaggcggcagcgcccaggtgaagctgcagcagagcggccctgatctggtgaagcctggcgccagcgtgaagatcagctgcaaggccagcggctacagcttcaccggctactacatgcactgggtgaaacagagccacggcaagagcctggaatggatcggcagagtgaaccccaatagcggcggcaccagctacaaccagaagttcaaggacaaggccatcctgaccgtggacaagagcagcagcaccgcctacatggaactgcggagcctgaccagcgaggacagcgccgtgtactactgcgcccggtccaagggcaactacttctacgccatggactactggggccagggcaccaccgtgaccgtgtctagcagcggcggcggcggaagcgaggttcagctggtggagtctggcggtggcctggtgcagccagggggctcactccgtttgtcctgtgcagcttctggctactcctttaccggctacactatgaactgggtgcgtcaggccccaggtaagggcctggaatgggttgcactgattaatccttataaaggtgtttccacctataaccagaaattcaaggatcgtttcacgatatccgtagataaatccaaaaacacagcctacctgcaaatgaacagcctgcgtgctgaggacactgccgtctattattgtgctagaagcggatactacggcgatagcgactggtattttgacgtctggggtcaaggaaccctggtcaccgtctcctcgggtggaggcggttcaggcggaggtggctctggcggtggcggatcggatatccagatgacccagtccccgagctccctgtccgcctctgtgggcgatagggtcaccatcacctgtcgtgccagtcaggacatccgtaattatctcaactggtatcaacagaaaccaggaaaagctccgaaactactgatttactatacctcccgcctggagtctggagtcccttctcgcttctctggttctggttctgggacggattacactctgaccatcagcagtctgcaaccggaggacttcgcaacttattactgtcagcaaggtaatactctgccgtggacgttcggacagggcaccaaggtggagatcaaagagcagaagctgattagcgaggaagatctggattataaggacgacgatgacaaatgagaattc

SEQ ID NO: 37 - protein, 520 aa, synthetic construct
TGMDIELTQSPSSLAVSLGQRATISCRASESVDSHGTSLMHWYQQKPGQPPKFLIYRASNLESGIPARFSGSGSRTDFTLTINPVETDDVAIYYCQQSNEDPPTFGGGTKLELKEGGGSGGGGSGGGGSAQVKLQQSGPDLVKPGASVKISCKASGYSFTGYYMHWVKQSHGKSLEWIGRVNPNSGGTSYNQKFKDKAILTVDKSSSTAYMELRSLTSEDSAVYYCARSKGNYFYAMDYWGQGTTVTVSSSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKEQKLISEEDLDYKDDDDKEF

SEQ ID NO: 38 - protein, 8 aa, synthetic construct
DYKDDDDK

SEQ ID NO: 39 - protein, 10 aa, synthetic construct
EQKLISEEDL

SEQ ID NO: 40 - DNA, 1452 bp, synthetic construct
  source 1..1452: mol_type = other DNA
gcggccgcaaactacaagacagacttgcaaaagaaggcatgcacagctcagcactgctctgttgcctggtcctcctgactggggtgagggccgaggtgcagctggtggagagcggggggggactggtgcagccaggaggaagcctgagactgagctgtaaggcttcaggatacagcttcacagggtactatatgcactgggtgagacaggcccccggaaaaggactggagtggatcggcagagtgaaccccaacagcggaggcaccagctacaaccagaaattcaaggaccgggccaccctgagcgtggacaacagcaaaaacacagcctacctgcagatgaacagcctgcgggccgaggacaccgccgtgtactattgtgccagaagcaagggcaactacttctacgccatggactattggggccagggaacactggtgaccgtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgattctaga

SEQ ID NO: 41 - protein, 450 aa, synthetic construct
EVQLVESGGGLVQPGGSLRLSCKASGYSFTGYYMHWVRQAPGKGLEWIGRVNPNSGGTSYNQKFKDRATLSVDNSKNTAYLQMNSLRAEDTAVYYCARSKGNYFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - DNA, 756 bp, synthetic construct
  source 1..756: mol_type = other DNA
gcggccgcaaactacaagacagacttgcaaaagaaggcatgcacagctcagcactgctctgttgcctggtcctcctgactggggtgagggccgacattcagatgacccagagccccagcagcctgagcgccagcgtgggagacagagtgaccatcacctgcagagccagcgagagcgtggacagccatggcacctcctttatgcactggtaccagcagaagcccggcaaggcccccaaattcctgatttacagagccagcaacctggagagcggcatcccctcaagattctcaggcagcggaagcggaaccgacttcacactgactatcagcagcgtgcagcccgaagattttgctgtgtactactgccagcagagcaacgaagatcctcctaccttcgggggcggcactaaggtggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagttcgcccgtcacaaagagcttcaacaggggagagtgttgattctaga

SEQ ID NO: 43 - protein, 218 aa, synthetic construct
DIQMTQSPSSLSASVGDRVTITCRASESVDSHGTSFMHWYQQKPGKAPKFLIYRASNLESGIPSRFSGSGSGTDFTLTISSVQPEDFAVYYCQQSNEDPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - DNA, 766 bp, synthetic construct
  source 1..766: mol_type = other DNA
atggcccaggtgcaactgcagcagtcaggggctgagcttgtgaagcctggggcttcagtgaagatgtcctgcaaggcttctggctacaccttcaccagctactggataacctgggtgaagcagaggcctggacaaggccttgagtggattggagatatttatcctggtagtggtagtactaactacaatgagaagttcaagagcaaggccacactgactgtagacacatcctccagcacagcctacatgcagctcagcagcctgacatctgaggactctgcggtctattactgtgcaagatggcgggacgggtactatgctatggactactggggccaagggaccacggtcaccgtctcctcaagtgaaggcggttcaggcggaggttgctctggcggtggcggatcggacatcgagctcactcagtctccaaccaccatggctgcatctcccggggagaagatcactatcacctgcagtgccagctcaagtataagttccaattacttgcattggtatcagcagaagccaggattctcccctaaactcttgatttataggacatccaatctggcttctggagtcccagctcgcttcagtggcagtgggtctgggacctcttactctctcacaattggcaccatggaagctgaagatgttgccacttactactgccagcagggtagtagtataccattcacgttcggctcggggacgaagttggagctgaaacgggcgggccgcaggtgcgccggtgccgtatccg

SEQ ID NO: 45 - protein, 255 aa, synthetic construct
MAQVQLQQSGAELVKPGASVKMSCKASGYTFTSYWITWVKQRPGQGLEWIGDIYPGSGSTNYNEKFKSKATLTVDTSSSTAYMQLSSLTSEDSAVYYCARWRDGYYAMDYWGQGTTVTVSSSEGGSGGGCSGGGGSDIELTQSPTTMAASPGEKITITCSASSSISSNYLHWYQQKPGFSPKLLIYRTSNLASGVPARFSGSGSGTSYSLTIGTMEAEDVATYYCQQGSSIPFTFGSGTKLELKRAGRRCAGAVS

SEQ ID NO: 46 - protein, 11 aa, synthetic construct
VRQAPGKGLEW

SEQ ID NO: 47 - protein, 16 aa, synthetic construct
LQMNSLRAEDTAVYYC

SEQ ID NO: 48 - DNA, 336 bp, synthetic construct
  source 1..336: mol_type = other DNA
gaggtccagctggtacagtctggggctgaggtgaagaagcctggggctacagtgaaaatctcctgcaaggtttctggcttcaacattaaagacacctatatgcactgggtgcaacaggcccctggaaaagggcttgagtggatgggaaggattgatcctgcgaatgataatactttatacgcagagaagttccagggcagagtcaccataaccgcggacacgtctacagacacagcctacatggagctgagcagcctgagatctgaggacacggccgtgtattactgtggtagaggttatggttactacgtatttgaccactggggccaaggcacc

SEQ ID NO: 49 - protein, 112 aa, synthetic construct
EVQLVQSGAEVKKPGATVKISCKVSGFNIKDTYMHWVQQAPGKGLEWMGRIDPANDNTLYAEKFQGRVTITADTSTDTAYMELSSLRSEDTAVYYCGRGYGYYVFDHWGQGT

SEQ ID NO: 50 - DNA, 306 bp, synthetic construct
  source 1..306: mol_type = other DNA
gacattgtgctgacccagtctccagcctccttggccgtgtctccaggacagagggccaccatcacctgcagagccagcaaaagtgtcagtacatctggctatagttatattcactggtatcagcagaaaccaggacaacctcctaaactcctgatttaccttgtatccaataaagacactggggtcccagccaggttcagcggcagtgggtctgggaccgatttcaccctcacaattaatcctgtggaagctaatgatactgctaaattattagctgtcagcacattatgggagcttacacgtcgg

SEQ ID NO: 51 - protein, 102 aa, synthetic construct
DIVLTQSPASLAVSPGQRATITCRASKSVSTSGYSYIHWYQQKPGQPPKLLIYLVSNKDTGVPARFSGSGSGTDFTLTINPVEANDTAKLLAVSTLWELTRR

SEQ ID NO: 52 - protein, 8 aa, synthetic construct
GYSFTGYY

SEQ ID NO: 53 - protein, 8 aa, synthetic construct
VNPNSGGT

SEQ ID NO: 54 - protein, 13 aa, synthetic construct
ARSKGNYFYAMDY

SEQ ID NO: 55 - protein, 12 aa, synthetic construct
ASESVDSHGTSF

SEQ ID NO: 56 - protein, 4 aa, synthetic construct
RASN

SEQ ID NO: 57 - protein, 9 aa, synthetic construct
QQSNEDPPT

(19) United States
(12) Patent Application Publication
(10) Pub. No.: US 2024/0374644 A1
(43) Pub. Date: Nov. 14, 2024

(54) HERV-K ANTIBODY THERAPEUTICS

(71) Applicant: SunnyBay BioTech, Inc., FREMONT, CA (US)

(72) Inventors: Feng WANG-JOHANNING, FREMONT, CA (US); Gary JOHANNING, FREMONT, CA (US)

(21) Appl. No.: 18/692,754

(22) PCT Filed: Sep. 16, 2022
(86) PCT No.: PCT/US2022/076624

§ 371 Date: Mar. 15, 2024

Related U.S. Application Data
(63) Continuation-in-part of application No. PCT/US2021/071505, filed on Sep. 18, 2021.

(51) Int. Cl. A61K 35/17; A61K 39/00; A61K 47/68; A61P 35/00; C07K 16/10; C07K 16/28; C12N 15/86; G01N 33/569

(52) U.S. Cl. CPC A61K 35/17; A61K 39/4611; A61K 39/4631; A61K 39/4632; A61K 39/464838; A61K 47/6841; A61P 35/00; C07K 16/1036; C07K 16/2809; C12N 15/86; G01N 33/56972; A61K 2039/505; C07K 2317/24; C07K 2317/31; C07K 2317/565; C07K 2317/622; C07K 2317/76; C12N 2740/15043; G01N 2333/15; G01N 2333/7051

(57) ABSTRACT

The invention provides therapeutic humanized anti-HERV-K antibodies, CAR, or a fusion thereof consisting of a bispecific T cell engager (BiTE) FOR CD3 and CDS, a DNA-encoded BiTE (DBiTE), or an antibody-drug conjugate (ADC). The invention also relates to peptides, proteins, nucleic acids, and cells for use in immunotherapeutic methods. In particular, the invention relates to the immunotherapy of cancer peptides bound to molecules of the MHC, or peptides as such, which can also be targets of antibodies and other binding molecules.

### FIELD OF THE INVENTION

This invention relates generally to cancer antigens.

### BACKGROUND OF THE INVENTION

Human endogenous retroviruses (HERVs) are well-known as genomic repeat sequences, with many copies in the genome. Approximately 8% of the human genome is of retroviral origin. See, Lander et al. Nature. 409, 860-921 (2001). Retroviruses typically lose infectivity because of the accumulation of genetic mutations. These genes are predominantly silent and not expressed in normal adult human tissues, except during pathologic conditions such as cancer.

The most biologically active HERVs are members of the HERV-K family. HERV-K has a complete sequence capable of expressing all the elements needed for a replication-competent retrovirus but remain silent in normal cells. Larsson, Kato, & Cohen, Current Topics Microbiol. Immunol., 148, 115-132 (1989); Ono, Yasunaga, Miyata, & Ushikubo, J. Virol. 60, 589-598 (1986). The inventors and others have reported that, sometimes, such as in tumors, expression of HERV-K is activated, and its envelope protein can be detected in several types of tumors at much higher levels than in normal tissues. See International Pat. Publ. WO 2010/138803 (Board of Regents, the University of Texas System); Wang-Johanning et al., Cancer Res., 77, Abstract nr LB-221 (2017), Johanning et al., Expression of human endogenous retrovirus-K is strongly associated with the basal-like breast cancer phenotype. Sci. Rep., 7, 41960 (2017); and Li et al., Clinical Cancer Research (2017). This association indicates that HERV-K could be an excellent tumor associated antigen and an ideal target for cancer immunotherapy. HERV-K is expressed in tumors and is absent in normal tissues, which minimizes off-target effects.

An important consideration in developing a cancer therapeutic is the expression profile of the tumor associated antigen. HERV-K is transcriptionally active in cancer tissues and cell lines. The inventors specifically identified HERV proteins and sequences in cancer cell lines and patient tumors. The inventors observed the expression of HERVs, especially HERV-K sequences, in breast, lung, prostate, ovarian, colon, pancreatic, and other solid tumors. They also found that the expression of HERV-K env transcripts in breast cancer was specifically associated with basal breast cancer, an aggressive subtype. Johanning et al., Expression of human endogenous retrovirus-K is strongly associated with the basal-like breast cancer phenotype. Sci. Rep., 7, 41960 (2017).

Several diagnostic products can be used as companion diagnostics for patient selection. One strategy targets endogenous viral antigens found only on cancer cells—not on normal tissues. The inventors' group discovered that both HERV-K RNAs (env or gag) and anti-HERV-K antibodies appear in the circulation of cancer patients.

An improved understanding of the tumor microenvironment of breast cancer is important for the design of rational and efficient therapy. One problem that has limited the success of therapy against solid tumors is the absence of tumor antigens highly expressed in tumor cells but not normal cells.

In the inventors' previous work, they showed that the HERV-K Env protein is commonly expressed on the surface of breast cancer cells. Wang-Johanning et al., J. Natl. Cancer Inst., 104, 189-210 (2012). Epithelial-mesenchymal transition (EMT) lowers infiltration of CD4 or CD8 T cells in some tumors. Chae et al., Science Reports, 8, 2918 (2018). HERV-K expression was demonstrated to induce epithelial-mesenchymal transition, leading to an increase in cell motility, both of which favor tumor dissemination. See Lemaitre et al., PLoS Pathog., 13, e1006451 (2017). Overexpression of HERV-K leads to cancer onset and contributes to cancer progression.

### SUMMARY OF THE INVENTION

The inventors found that checkpoint molecule levels in serum and tumor-infiltrating lymphocytes (TILs) are highly correlated to HERV-K antibody titers, especially in aggressive breast cancer patients, e.g., patients with invasive ductal carcinoma (IDC) or invasive mammary carcinoma (IMC). The phenotypic and functional characteristics of tumor-infiltrating lymphocytes in breast cancer are related to HERV-K status. The combination of checkpoint inhibition and HERV-K antibody therapy should result in better killing efficacy.

In a first embodiment, the invention provides therapeutic humanized anti-HERV-K antibodies The invention also provides a fusion therapeutic humanized anti-HERV-K antibody of a bispecific T cell engager (BiTE) for CD3 or CD8, a DNA-encoded BiTE (DBiTE), or an antibody-drug conjugate (ADC). Cancer cells overexpressing HERV-K can be good targets and good models for the anti-HERV-K humanized antibodies and antibody-drug conjugates of the invention, because more antibodies may be bound per cell.

In a second embodiment, the invention provides a humanized antibody clones (HUM1) generated from bacteria. The invention also provides humanized antibody generated from mammalian cells (hu6H5). Antibodies from both these groups bind antigens produced from recombinant HERV-K Env surface fusion protein (KSU) and lysates from MDA-MB-231 breast cancer cells. The hu6H5 generated from mammalian cells was compared with our other forms of anti-HERV-K antibodies. The hu6H5 has binding affinity to HERV-K antigen that is similar to murine antibodies (m6H5), chimeric antibodies (cAb), or humanized antibody (HUM1). The hu6H5 antibody induces cancer cells to undergo apoptosis, inhibits cancer cell proliferation, and kills cancer cells that express HERV-K antigen. The hu6H5 antibody was demonstrated to reduce tumor viability in mouse MDA-MB-231 xenografts, and notably reduced cancer cell metastasis to lung and lymph nodes. Mice bearing human breast cancer tumors treated with these humanized antibodies had prolonged survival compared to control mice that did not receive antibody treatment.

In a third embodiment, the invention provides HERV-K env gene generated from a breast cancer patient as an oncogene which can induce cancer cell proliferation, tumor growth, and metastasis to lungs and lymph nodes. Cells expressing HERV-K showed reduced expression of genes associated with tumor suppression, including Caspase 3, Caspase 9, pRB, SIRT-1, and CIDEA. Cells expressing HERV-K showed increased expression of genes associated tumor formation, including Ras, p-ERK, P-P-38, and beta Catenin.

In a fourth embodiment, the invention provides BiTEs directed against T cell CD3 or CD8 and the tumor-associated antigen HERV-K. The inventors produced such a BiTE, which comprised antibodies targeting either CD3 or CD8 and HERV-K (VL-VH 6H5scFv-VH-VLhuCD3 or CD8+c-myc+FLAG) or (VL-VH hu6H5scFv-VH-VLhuCD3 or huCD8+c-myc+FLAG). FLAG-tag, a peptide recognized by an antibody (DYKDDDDK) (SEQ ID NO: 38) and Myc-tag, a short peptide recognized by an antibody (EQKLISEEDL) (SEQ ID NO: 39).

In a fifth embodiment, the invention provides T cells expressing a lentiviral CAR expression vector that bears a humanized or fully human HERV-K scFv. See FIG. 1. These T-cells effectively lyse and kill tumor cells from several cancers. Humanized K-CARs expressed from lentiviral vectors are pan-cancer CAR-Ts.

In a sixth embodiment, the invention provides humanized single chain variable fragment (scFv) antibody. This antibody can bind antigens produced from recombinant HERV-K Env surface fusion protein (KSU) and lysates from MDA-MB-231 breast cancer cells. A CAR produced from this humanized scFv can be cloned into a lentiviral vector. This recombinant vector can be used in combination with therapies, including but are not limited to K-CAR T cells plus checkpoint inhibitors, proinflammatory cytokines such as interleukin (IL)-12 and IL-18, oncolytic viruses, and kinase inhibitors. The kinase inhibitors include but not limited to p-RSK and p-ERK.

In a seventh embodiment, the invention provides HERV-K staining that overlaps in many cases with staining of the serum tumor marker CK. HERV-K can be a CTC marker and a target for HERV-K antibody therapy.

In an eighth embodiment, the invention provides HERV-K as a stem cell marker. Targeting of HERV-K can block tumor progression by slowing or preventing growth of cancer stem cells. Targeting of HERV-K with circulating therapeutic antibodies or other therapies can also kill CTCs and prevent metastasis of these circulating cells to distant sites.

In a ninth embodiment, the invention provides that forced overexpression of HERV-K with agents that induce expression of HERV-K by innate immune response (such as Poly I:C treatment) or LTR hypomethylation (such as by 5-Aza) provokes cancer cells to increase production of a target that would make them more susceptible to targeted therapy to include targeted immunotherapy.

In a tenth embodiment, the invention improves an in vivo enrichment technique (IVE: ≈20-fold enhancement) in SCID/beige mice, allowing for rapid expansion and B cell activation. This improved technique can produce many antigen-specific plasmablasts. For donors with cancer with a higher titer of antibodies, the improved technique uses a protocol with humanized mice (HM) or human tumor mice (HTM) instead of the standard SCID/beige mice. For normal donors without cancer and who have no memory B cells, the improved technique uses a protocol with modifications: Mice are treated with cytokine cocktails (days 1, 7, and 14). Mice are then boosted by antigens on days 14 and 21. Sera are collected from mice and binding affinity is tested by ELISA. After increased antibody titers are detected, spleens are harvested, analyzed, and used to make hybridomas. Higher antibody titers were detected in mice using an in vivo enrichment protocol.

In an eleventh embodiment, the invention provides a method to determine cells that not only produce antibodies but can also bind antigen and kill cancer cells. This method can efficiently stimulate and expand CD40-B cells to large numbers in high purity (>90%) and induce secretion of their antibodies.

In a twelfth embodiment, the invention provides a method of post-incubation of treated B cells. Glass cover slips are washed and tagged with fluorescent anti-human IgG antibody and read using a microengraving technology to reveal discrete spots that correspond to secretion of antigen-specific antibodies by single B cells.

In a thirteenth embodiment, the invention provides for the development of a platform to determine the binding kinetics and cell-to-cell interactions of every cell in a microwell slab.

In a fourteenth embodiment, the invention strikingly provides significantly enhanced expression of six circulating immune checkpoint proteins in the plasma of breast cancer patients. The invention also provides a marked drop in immune checkpoint protein levels in patients at 6 months or 18 months post-surgery vs. pre-surgery. A positive association between soluble immune checkpoint protein molecule levels and HERV-K antibody titers induced by HERV-K expression in the tumor results. HERV-K antibody titers can influence immune checkpoint protein levels in breast cancer. Thus, the expression of HERV-K can control the immune responses of breast cancer patients.

In another aspect, these findings collectively show that the immunosuppressive domain (ISD) of HERV-K is a yet unrecognized immune checkpoint on cancer cells, analogous to the PD-L1 immune checkpoint. In a fifteenth embodiment, the invention provides that blockade of the ISD with immune checkpoint inhibitors of HERV-K, including but not limited to monoclonal antibodies and drugs targeting the ISD of HERV-K, is a cancer immunomodulator therapy that will allow T cells to continue working and unleash immune responses against cancer and enhance existing responses, to promote elimination of cancer cells.

In a sixteenth embodiment, the invention provides humanized and fully human (hTab) antibodies targeting HERV-K. These antibodies enhance checkpoint blockade antibody treatment efficacy. Effective combined cancer therapies include but are not limited to combinations of (a) HERV-K humanized or hTAb (1.5 mg/kg), (b) K-CAR, (c) K-BiTE, (d) HERV-K shRNAs or CRISPR/Cas9 genome editing technology to knock down HERV-K gene expression, (e) or preventive or therapeutic HERV-K vaccines, including full-length and truncated HERV-K Env proteins and HERV-K Env peptides. Effective combined cancer therapies include full-length and truncated HERV-K Env proteins and HERV-K Env peptides, combined with factors including but not limited to (a) anti-ICP antibody, (b) cancer chemotherapy, (c) 5-Azacytidine, 5-aza-2′-deoxycytidine, or other epigenetic modulating agents, such as DNA methyltransferase inhibitors (DNMTi) and histone deacetylase inhibitors (HDACi), (d) EMT inhibitors, (e) inhibitors of cell migration or invasion,(f) induction of S or G2 phase cell cycle arrest, (g) inhibitors of PI3K/AKT/mTOR or MAPK/ERK signaling pathways, or (f) signal transduction to HIF1α.

In a seventeenth embodiment, the invention provides humanized antibodies targeting HERV-K that can be used for antibody-drug conjugates to deliver the drugs into cancer cells and tumors.

In an eighteenth embodiment, the invention provides antibodies targeting HERV-K that can be used for tumor imaging.

In a nineteenth embodiment, the invention provides a new CAR using hu6H5 scFv.

In a twenty embodiment, the invention provides a new BITE using hu6H5 scFv including CD3 BiTEs and CD8 BiTEs.

### BRIEF DESCRIPTION OF THE DRAWINGS

FIG. 1 is a diagram of a lentiviral CAR expression vector (pWPT-GFP vector with psPAX2 and pMD2g) that bears a humanized or fully human HERV-K scFv.

FIG. 2 shows an ELISA used for comparing binding to the HERV-K env target of chimeric 6H5, HUM1 (generated from bacteria), and new hu6H5 (new humanized anti-HERV-K antibody generated from mammalian cells). 1000, 1:1000 dilution; 2000, 1:2000 dilution; 4000, 1:4000 dilution; 8000, 1:8000 dilution.

FIG. 3 shows an MTS assay used to determine inhibition of proliferation of cells treated with hu6H5. Significantly reduced cell proliferation was observed in cells treated with either of the 6H5 antibodies (human or mouse). Inhibition was more prominent in 231K cells that express higher levels of HERV-K than 231C cells that did not express higher HERV-K levels. 231K cells are MDA-MB-231 human breast cancer cells stably transduced with HERV-K full length viral envelope gene protein by a retroviral vector and were evaluated in comparison with control non-transduced cells (231C) regarding cell proliferation, cell killing, and apoptosis.

FIG. 4. CD3BiTE mediated secretion of IFNγ from normal donor peripheral blood mononuclear cells (PBMCs) in the presence of MDA-MB-231 luc cells. 5×10³ cells/well were seeded in 96-well plate. PBMCs from ND #230341 (positive control) and four normal donors were used as effector cells. The ratio of the effector/tumor cells was 10/1. 140 μg/ml of CD3BiTE was used. Seventy-two hours after plate set-up, the supernatant was harvested for IFNγ assay.

FIG. 5 is a pair of bar graphs. FIG. 5A shows significantly increased killing of cancer cells was demonstrated by an LDH release assay in the supernatant of effector: tumor cells (10:1) treated with K3Bi at 0 ng/ml+PBMC or 100 ng/ml+PBMC for seventy-two hours. FIG. 5B shows that IFNγ secretion was significantly increased in three breast cancer cell lines treated with K3Bi (100 ng/ml) for seventy-two hours. Untreated cells, peripheral blood mononuclear cell only, or BiTE only were used as controls.

FIG. 6 is a set of line graphs showing the efficacy of a bispecific T cell engager (BiTE) targeting HERV-K. NOD/SCID/IL-2Rγnull (NSG) mice were inoculated with MDA-MB-231 HERV-K-positive breast cancer cells on day zero and dosed with peripheral blood mononuclear cells or BiTE on the days indicated. Tumor volumes were calculated throughout the assay by measuring tumor volumes using a caliper.

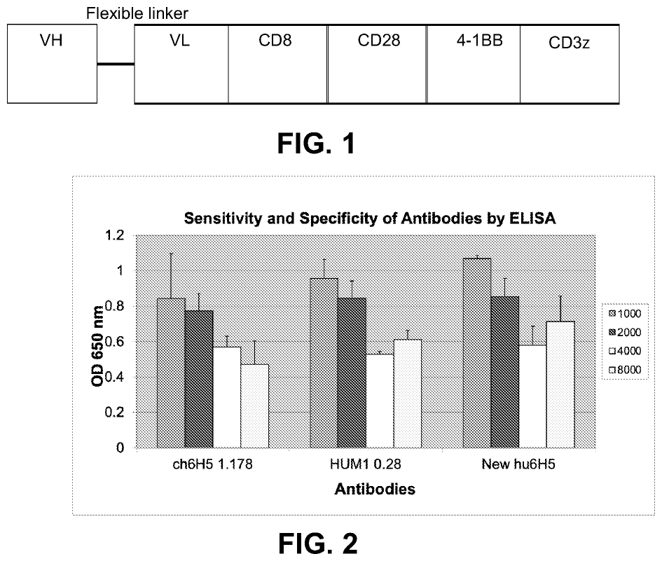

### DETAILED DESCRIPTION OF THE INVENTION

### Utility of the Invention

This specification provides methods for generated a humanized anti-HERV-K antibody. Anti-tumor effects of hu6H5 were demonstrated in vitro and in vivo.

This invention provides methods for treating patients suffering from cancer. In a twentieth embodiment, the invention provides to a method of treating cancer comprising administering a therapeutic humanized anti-HERV-K antibody or a fusion thereof consisting of a CAR, a BiTE or an antibody-drug conjugate, and optionally combine with one or more immune checkpoint blockers. Each therapeutics individually target the immune system. In a twenty-first embodiment, the methods of the invention inhibit metastases. In a twenty-second embodiment, the methods of the invention reduce tumor size. In a twenty-third embodiment, the methods of the invention inhibit the growth of tumor cells. In a twenty-fourth embodiment, the methods of the invention detect cancer and cancer metastasis.

### Definitions

For convenience, the meaning of some terms and phrases used in the specification, examples, and appended claims, are listed below. Unless stated otherwise or implicit from context, these terms and phrases have the meanings below. These definitions are to aid in describing particular embodiments and are not intended to limit the claimed invention. Unless otherwise defined, all technical and scientific terms have the same meaning as commonly understood by one of ordinary skill in the molecular biology art. For any apparent discrepancy between the meaning of a term in the art and a definition provided in this specification, the meaning provided in this specification shall prevail.

5-Aza has the biotechnological art-recognized meaning of 5-azacytidine.

6H5 has the biotechnological art-recognized meaning of a murine anti-HERV-K monoclonal antibody developed in our laboratory.

About has the biotechnological art-recognized meaning and varies depending on the context in which the term is used. If there are uses of the term which are not clear to persons of ordinary skill in the biotechnological art given the context in which it is used, about will mean up to plus or minus 10% of the value.

Antibody-drug conjugate (ADC) has the biotechnological art-recognized meaning of highly potent biological drugs built by attaching a small molecule anticancer drug or another therapeutic agent to an antibody, with either a permanent or a labile linker. The antibody targets a specific antigen only found on target cells.

B7 family has the biotechnological art-recognized meaning of inhibitory ligands with undefined receptors. The B7 family encompasses B7-H3 and B7-H4, both upregulated on tumor cells and tumor infiltrating cells. The complete hB7-H3 and hB7-H4 sequence can be found under GenBank Accession Nos. Q5ZPR3 and AAZ17406, respectively.

BiTE has the biotechnological art-recognized meaning of a bispecific T cell engager. A BiTE means a recombinant bispecific protein with two linked scFvs from two antibodies, one targeting a cell-surface molecule on T cells (for example, CD3ε) and the other targeting antigens on the surface of malignant cells. The two scFvs are linked by a short flexible linker. The term DNA-encoded BiTE (DBiTE) includes any BiTE-encoding DNA plasmid that can be expressed in vivo.

Cancer antigen or tumor antigen has the biotechnological art-recognized meaning of (i) tumor-specific antigens, (ii) tumor-associated antigens, (iii) cells that express tumor-specific antigens, (iv) cells that express tumor-associated antigens, (v) embryonic antigens on tumors, (vi) autologous tumor cells, (vii) tumor-specific membrane antigens, (viii) tumor-associated membrane antigens, (ix) growth factor receptors, (x) growth factor ligands, and (xi) any other type of antigen or antigen-presenting cell or material associated with a cancer.

Combination therapy has the medical art-recognized meaning and embraces administration of each agent or therapy in a sequential manner in a regimen that will provide beneficial effects of the combination, and co-administration of these agents or therapies in a substantially simultaneous manner, such as in a single capsule having a fixed ratio of these active agents or in multiple, separate capsules for each agent. Combination therapy also includes combinations where individual elements can be administered at separate times and/or by different routes, but which act in combination to provide a beneficial effect by co-action or pharmacokinetic and pharmacodynamics effect of each agent or tumor treatment approaches of the combination therapy.

CTL has the biotechnological art-recognized meaning of cytolytic T cell or cytotoxic T cell.

Cytotoxic T Lymphocyte Associated Antigen-4 (CTLA-4) has the biotechnological art-recognized meaning of a T cell surface molecule that is a member of the immunoglobulin superfamily. This protein downregulates the immune system by binding to CD80 and CD86. The term CTLA-4 includes human CTLA-4 (hCTLA-4), variants, isoforms, and species homologs of hCTLA-4, and analogs having at least one common epitope with hCTLA-4. The complete hCTLA-4 sequence can be found under GenBank Accession No. P16410.

DCIS has the biotechnological art-recognized meaning of ductal carcinoma in situ.

Derived from a designated polypeptide or protein has the biotechnological art-recognized meaning of the origin of the polypeptide. Preferably, the polypeptide or amino acid sequence derived from a particular sequence has an amino acid sequence essentially identical to that sequence or a portion thereof, wherein the portion consists of at least 10-20 amino acids, preferably at least 20-30 amino acids, more preferably at least 30-50 amino acids, or which is otherwise identifiable to one of ordinary skill in the in the molecular biological art as having its origin in the sequence. Polypeptides derived from another peptide can have one or more mutations relative to the starting polypeptide, e.g., one or more amino acid residues substituted with another amino acid residue, or which has one or more amino acid residue insertions or deletions. A polypeptide can comprise an amino acid sequence which is not naturally occurring. Such variants have less than 100% sequence identity or similarity with the starting molecule. In some embodiments, the peptides are encoded by a nucleotide sequence. Nucleotide sequences of the invention can be useful for several applications, including cloning, gene therapy, protein expression and purification, mutation introduction, DNA vaccination of a host in need thereof, antibody generation for, e.g., passive immunization, PCR, primer and probe generation, and the like.

Effector cell has the biotechnological art-recognized meaning of an immune cell involved in the effector phase of an immune response, as opposed to the cognitive and activation phases of an immune response. Exemplary immune cells include a cell of a myeloid or lymphoid origin, for instance lymphocytes (such as B cells and T cells including cytolytic T cells (CTLs)), killer cells, natural killer cells, macrophages, monocytes, eosinophils, polymorphonuclear cells, such as neutrophils, granulocytes, mast cells, and basophils. Some effector cells express specific Fc receptors (FcRs) and carry out specific immune functions.

Env has the biotechnological art-recognized meaning of the viral envelope protein. env has the biotechnological art-recognized meaning of the viral envelope RNA.

Epitope has the biotechnological art-recognized meaning of a protein determinant capable of specific binding to an antibody. Epitopes usually consist of surface groupings of molecules such as amino acids or sugar side chains and usually have specific three-dimensional structural characteristics and specific charge characteristics. Conformational and non-conformational epitopes are distinguished because the binding to the former but not the latter is lost in the presence of denaturing solvents. The epitope can comprise amino acid residues directly involved in the binding (also called immunodominant component of the epitope) and other amino acid residues, which are not directly involved in the binding, such as amino acid residues which are effectively blocked by the specifically antigen binding peptide (the amino acid residue is within the footprint of the specifically antigen binding peptide).

FACS has the biotechnological art-recognized meaning of fluorescence activated cell sorting.

Framework region has the biotechnological art-recognized meaning of a subdivision of the variable region (Fab) of an antibody. The variable region of an antibody is composed of seven amino acid regions, four of which are framework regions and three of which are hypervariable regions.

HERV has the biotechnological art-recognized meaning of human endogenous retrovirus. The human endogenous retrovirus (HERV) is a retrovirus present in the form of proviral DNA integrated into the genome of all normal cells and is transmitted by Mendelian inheritance patterns. HERV-X, where X is an English letter, has the biotechnological art-recognized meaning of other families of HERVs.

HERV-K has the biotechnological art-recognized meaning of the HERV-K family of endogenous retroviruses. HERV-K is expressed on many tumor types, including, but not limited to, melanoma, breast cancer (Wang-Johanning et al. (2003)), ovarian cancer (Wang-Johanning et al. (2007)), lymphoma, and teratocarcinoma. Infected cells, including those infected by HIV, also express HERV-K. This provides an attractive opportunity that one CAR design targeting HERV-K may be used to treat a variety of cancers and infections.

HM has the biotechnological art-recognized meaning of humanized mice.

HTM has the biotechnological art-recognized meaning of human tumor mice.

hTAb has the biotechnological art-recognized meaning of a fully human tumor antibody.

Human endogenous retrovirus-K, HERV-K, HERV, human endogenous retrovirus, endogenous retrovirus, and ERV include any variants, isoforms and species homologs of endogenous retroviruses naturally expressed by cells or are expressed on cells transfected with endogenous retroviral genes.

ICP has the biotechnological art-recognized meaning of immune checkpoint.

IDC has the biotechnological art-recognized meaning of invasive ductal carcinoma.

IHC has the biotechnological art-recognized meaning of immunohistochemistry.

ILC has the biotechnological art-recognized meaning of invasive lobular carcinoma.

Immune cell is a cell of hematopoietic origin and that plays a role in the immune response. Immune cells include lymphocytes (e.g., B cells and T cells), natural killer cells, and myeloid cells (e.g., monocytes, macrophages, eosinophils, mast cells, basophils, and granulocytes).

Immune checkpoint blocker has the biotechnological art-recognized meaning of a molecule that totally or partially reduces, inhibits, interferes with, or modulates one or more checkpoint proteins. In some embodiments, the immune checkpoint blocker prevents inhibitory signals associated with the immune checkpoint. In some embodiments, the immune checkpoint blocker is an antibody, or fragment thereof that disrupts inhibitory signaling associated with the immune checkpoint. In some embodiments, the immune checkpoint blocker is a small molecule that disrupts inhibitory signaling. In some embodiments, the immune checkpoint blocker is an antibody, fragment thereof, or antibody mimic, that prevents the interaction between checkpoint blocker proteins, e.g., an antibody, or fragment thereof, that prevents the interaction between PD-1 and PD-L1. In some embodiments, the immune checkpoint blocker is an antibody, or fragment thereof, that prevents the interaction between CTLA-4 and CD80 or CD86. In some embodiments, the immune checkpoint blocker is an antibody, or fragment thereof, that prevents the interaction between LAG3 and its ligands, or TIM-3 and its ligands. The checkpoint blocker can also be in the soluble form of the molecules (or variants thereof) themselves, e.g., a soluble PD-L1 or PD-L1 fusion.

Immune checkpoint has the biotechnological art-recognized meaning of co-stimulatory and inhibitory signals that regulate the amplitude and quality of T cell receptor recognition of an antigen. In some embodiments, the immune checkpoint is an inhibitory signal. In some embodiments, the inhibitory signal is the interaction between PD-1 and PD-L1. In some embodiments, the inhibitory signal is the interaction between CTLA-4 and CD80 or CD86 to displace CD28 binding. In some embodiments the inhibitory signal is the interaction between LAG3 and MHC class II molecules. In some embodiments, the inhibitory signal is the interaction between TIM3 and galectin 9.

In vivo has the biotechnological art-recognized meaning of processes that occur in a living organism. The term mammal or subject or patient includes both humans and non-humans and includes, but is not limited to, humans, non-human primates, canines, felines, rodents, bovines, equines, and pigs.

Inhibits growth (e.g., referring to cells, such as tumor cells) has the biotechnological art-recognized meaning of any measurable decrease in the cell growth when contacted with HERV-K specific therapeutic agents as compared to the growth of the same cells not in contact with the HERV-K specific therapeutic agents, e.g., the inhibition of growth of a cell culture by at least about 10%, 20%, 30%, 40%, 50%, 60%, 70%, 80%, 90%, 99%, or 100%. Such a decrease in cell growth can occur by a variety of mechanisms exerted by the anti-HERV-K agents, either individually or in combination, e.g., apoptosis.

ISD has the biotechnological art-recognized meaning of immunosuppressive domain.

K-CAR or HERV-Kₑₙᵥ CAR has the biotechnological art-recognized meaning of a HERV-K envelope gene (surface or transmembrane) chimeric antigen receptor (CAR) genetic construct. The term HERV-Kenv CAR-T cells or K-CAR-T cells has the biotechnological art-recognized meaning of T cells transduced with a K-CAR or HERV-Kenv CAR lentiviral or Sleeping Beauty expression system.

KD has the biotechnological art-recognized meaning of knockdown, usually by an shRNA.

KSU has the biotechnological art-recognized meaning of HERV-K envelope surface fusion protein.

KTM has the biotechnological art-recognized meaning of HERV-K Env transmembrane protein.

Linked, fused, or fusion, are used interchangeably. These terms has the biotechnological art-recognized meaning of the joining of two more elements or components or domains, by whatever means including chemical conjugation or recombinant means. Methods of chemical conjugation (e.g., using heterobifunctional crosslinking agents) are known in the art.

Linker or linker domain has the biotechnological art-recognized meaning of a sequence which connects two or more domains (e.g., a humanized antibody targeting HERV-K and an antibody targeting a T cell protein) in a linear sequence. The constructs suitable for the methods disclosed can use one or more linker domains, such as polypeptide linkers.

Lymphocyte Activation Gene-3 (LAG3) has the biotechnological art-recognized meaning of an inhibitory receptor associated with inhibition of lymphocyte activity by binding to MHC class II molecules. This receptor enhances the function of Treg cells and inhibits CD8+ effector T cell function. LAG3 includes human LAG3 (hLAG3), variants, isoforms, and species homologs of hLAG3, and analogs having at least one common epitope. The complete hLAG3 sequence can be found under GenBank Accession No. P18627.

Mammosphere has the biotechnological art-recognized meaning of breast or mammary cells cultured under non-adherent non-differentiating conditions that form discrete clusters of cells.

MDA-MB-231 pLVXC or 231-C refer to MDA-MB-231 cells transduced with pLVXC.

MDA-MB-231 pLVXK or 231-K refer to MDA-MB-231 cells transduced with pLVXK.

Nucleic acid has the biotechnological art-recognized meaning of deoxyribonucleotides or ribonucleotides and polymers thereof in either single-stranded or double-stranded form. Unless specifically limited, encompasses nucleic acids containing known analogues of natural nucleotides with similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences and the sequence explicitly indicated. Specifically, degenerate codon substitutions can be achieved by generating sequences in which the third position of one or more selected (or all) codons is substituted with mixed-base and/or deoxyinosine residues. See Batzer et al., Nucleic Acid Res., 19, 5081 (1991); Ohtsuka et al., Biol. Chem., 260, 2605-2608 (1985); and Rossolini et al., Mol. Cell. Probes, 8, 91-98 (1994). For arginine and leucine, modifications at the second base can also be conservative. The term nucleic acid is used interchangeably with gene, cDNA, and mRNA encoded by a gene.

PBMC has the biotechnological art-recognized meaning of peripheral blood mononuclear cell.

PDX has the biotechnological art-recognized meaning of a patient derived xenograft. A PDX is typically produced by transplanting human tumor cells or tumor tissues into an immunodeficient murine model of human cancer.

Percent identity, in the context of two or more nucleic acid or polypeptide sequences, refer to two or more sequences or subsequences with a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent identity can exist over a region of the sequence being compared, e.g., over a functional domain or exist over the full length of the two sequences to be compared. For sequence comparison, typically one sequence acts as a reference sequence to which test sequences are compared. When using a sequence comparison algorithm, test and reference sequences are input into a computer, subsequence coordinates are designated, and sequence algorithm program parameters are designated. The sequence comparison algorithm then calculates the percent sequence identity for the test sequences relative to the reference sequence, based on the designated program parameters. Optimal alignment of sequences for comparison can be conducted, e.g., by the local homology algorithm of Smith & Waterman, Adv. Appl. Math., 2, 482 (1981), by the homology alignment algorithm of Needleman & Wunsch, J. Mol. Biol., 48, 443 (1970), by the search for similarity method of Pearson & Lipman, Proc. Natl. Acad. Sci., U.S.A., 85, 2444 (1988), by computerized implementations of these algorithms (GAP, BESHERV-KIT, FASTA, and HERV-KASTA in the Wisconsin Genetics Software Package, Genetics Computer Group, 575 Science Dr., Madison, WI, USA), or by visual inspection. One example of an algorithm suitable for determining percent sequence identity and sequence similarity is the BLAST algorithm, which is described in Altschul et al., J. Mol. Biol. 215, 403-410 (1990). Software for performing BLAST analyses is publicly available through the National Center for Biotechnology Information website.

Pharmaceutically acceptable generally means those compounds, materials, compositions, and/or dosage forms which are, within the scope of sound medical judgment, suitable for use in contact with the tissues, organs, and/or bodily fluids of humans and animals without excessive toxicity, irritation, allergic response, or other problems or complications commensurate with a reasonable benefit/risk ratio.

pLVXC has the biotechnological art-recognized meaning of a control expression vector only.

pLVXK has the biotechnological art-recognized meaning of an HERV-K expression vector.

Polypeptide linker has the biotechnological art-recognized meaning of a peptide or polypeptide sequence (e.g., a synthetic peptide or polypeptide sequence) which connects two or more domains in a linear amino acid sequence of a polypeptide chain. Such polypeptide linkers can provide flexibility to the polypeptide molecule. The polypeptide linker can connect (e.g., genetically fuse) one or more Fc domains and/or a drug.

Programmed Death Ligand-1 (PD-L1) is one of two cell surface glycoprotein ligands for PD-1 (the other being PD-L2) that downregulates T cell activation and cytokine secretion upon binding to PD-1. PD-L1 includes human PD-L1 (hPD-L1), variants, isoforms, and species homologs of hPD-L1, and analogs having at least one common epitope with hPD-L1. The complete hPD-L1 sequence can be found under GenBank Accession No. Q9NZQ7.

Programmed Death-1 (PD-1) receptor has the biotechnological art-recognized meaning of an immuno-inhibitory receptor belonging to the CD28 family. PD-1 is expressed predominantly on previously activated T cells in vivo, and binds to two ligands, PD-L1 and PD-L2. PD-1 includes human PD-1 (hPD-1), variants, isoforms, and species homologs of hPD-1, and analogs having at least one common epitope with hPD-1. The complete hPD-1 sequence can be found under GenBank Accession No. AAC51773.

Recombinant host cell (or simply host cell) has the biotechnological art-recognized meaning of a cell into which an expression vector was introduced. Such terms refer not only to the particular subject cell, but also to the progeny of such a cell. Because some modifications can occur in succeeding generations due to either mutation or environmental influences, such progeny might not be identical to the parent cell but are still included within the scope of host cell. Recombinant host cells include, for example, transfectomas, such as CHO cells, HEK293 cells, NS/0 cells, and lymphocytic cells.

scFv has the biotechnological art-recognized meaning of single chain variable fragment.

SU has the biotechnological art-recognized meaning of the HERV-K surface fusion protein.

Sufficient amount or amount sufficient to means an amount sufficient to produce a desired effect, e.g., an amount sufficient to reduce the size of a tumor.

Synergy or synergistic effect regarding an effect produced by two or more individual components has the biotechnological art-recognized meaning of a phenomenon in which the total effect produced by these components, when utilized in combination, is greater than the sum of the individual effects of each component acting alone.

T cell has the biotechnological art-recognized meaning of a CD4+ T cell or a CD8+ T cell. The term T cell encompasses TH1 cells, TH2 cells and TH17 cells.

T Cell Membrane Protein-3 (TIM3) is an inhibitory receptor involved in the inhibition of lymphocyte activity by inhibition of TH1 cells responses. Its ligand is galectin 9, which is upregulated in various types of cancers. TIM3 includes human TIM3 (hTIM3), variants, isoforms, and species homologs of hTIM3, and analogs having at least one common epitope. The complete hTIM3 sequence can be found under GenBank Accession No. Q8TDQo.

Therapeutically effective amount is an amount that is effective to ameliorate a symptom of a disease. A therapeutically effective amount can be a prophylactically effective amount as prophylaxis can be considered therapy.

TM has the biotechnological art-recognized meaning of the HERV-K transmembrane protein.

TNBC has the biotechnological art-recognized meaning of triple-negative breast cancer.

Transgenic non-human animal has the biotechnological art-recognized meaning of a non-human animal having a genome comprising one or more human heavy and/or light chain transgenes or transchromosomes (either integrated or non-integrated into the animal's natural genomic DNA) and which can express fully human antibodies. A transgenic mouse can have a human light chain transgene and either a human heavy chain transgene or human heavy chain transchromosome, such that the mouse produces human anti-HERV-K antibodies when immunized with HERV-K antigen and/or cells expressing HERV-K. The human heavy chain transgene can be integrated into the chromosomal DNA of the mouse, as for transgenic mice, for instance HuMAb mice or the human heavy chain transgene can be maintained extrachromosomally, as for transchromosomal KM mice as described in WO02/43478. Such transgenic and transchromosomal mice (collectively referred to herein as transgenic mice) can produce multiple isotypes of human mAbs to an antigen (such as IgG, IgA, IgM, IgD, or IgE) by undergoing V-D-J recombination and isotype switching. Transgenic, nonhuman animal can also be used for production of antibodies against a specific antigen by introducing genes encoding such specific antibody, for example by operatively linking the genes to a gene expressed in the milk of the animal.

Treatment means the administration of an effective amount of a therapeutically active compound of the invention to ease, ameliorating, arresting, or eradicating (curing) symptoms or disease states.

Vector has the biotechnological art-recognized meaning of a nucleic acid molecule capable of transporting another nucleic acid to which it was linked. One type of vector is a plasmid, which has the biotechnological art-recognized meaning of a circular double-stranded DNA loop into which additional DNA segments can be ligated. Another type of vector is a viral vector, wherein additional DNA segments can be ligated into the viral genome. Some vectors are capable of autonomous replication in a host cell into which they are introduced (for instance bacterial vectors having a bacterial origin of replication and episomal mammalian vectors). Other vectors (such as non-episomal mammalian vectors) can be integrated into the genome of a host cell upon introduction into the host cell, and thereby are replicated along with the host genome. Some vectors can direct the expression of genes to which they are operatively linked. Such vectors are referred to herein as recombinant expression vectors (or expression vectors). Expression vectors useful in recombinant DNA techniques are often in plasmids. In this specification, the terms plasmid and vector are used interchangeably as the plasmid is the most commonly used form of vector. However, the invention is intended to include such other forms of expression vectors, such as viral vectors (such as replication-defective retroviruses, adenoviruses, and adeno-associated viruses), which serve equivalent functions.

Unless otherwise defined, scientific and technical terms used with this application shall have the meanings commonly understood by persons having ordinary skill in the biomedical art. This invention is not limited to the particular methodology, protocols, reagents, etc., described herein and can vary.

The disclosure described herein does not concern a process for cloning humans, methods for modifying the germ line genetic identity of humans, uses of human embryos for industrial or commercial purposes, or procedures for modifying the genetic identity of animals likely to cause them suffering with no substantial medical benefit to man or animal and animals resulting from such processes.

### Cancer Therapeutic Antibodies

Developing cancer therapeutic antibodies, such as Herceptin® (trastuzumab), Avastin® (bevacizumab), Erbitux® (cetuximab), and others saved many tens of thousands of lives worldwide. In particular, the treatment of HER2-positive metastatic breast or ovarian cancer using trastuzumab has dramatically changed patient outcomes. Antibody therapeutics offer distinct advantages relative to small molecule drugs, namely: (i) defined mechanisms of action; (ii) higher specificity and fewer-off target effects; and (iii) predictable safety and toxicological profiles. As extensive studies with anti-Her2 and anti-EGFR monoclonals attest, only a few antibodies out of many thousands identified based on their ability to bind to their molecular target with high affinity exhibit properties required for clinically effective cancer cell killing. The efficacy of therapeutic antibodies results primarily from their ability to elicit potent tumor cytotoxicity either via direct induction of apoptosis in target cells or through effector-mediated functions like antibody dependent cell-mediated cytotoxicity (ADCC) and complement dependent cytotoxicity (CDC).

The major methodologies for antibody isolation are: (i) in vitro screening of libraries from immunized animals or from synthetic libraries using phage or microbial display 43-45, and (ii) isolation of antibodies following B cell immortalization or cloning 46-48. These methodologies suffer from either or both of these drawbacks, severely limiting the numbers of unique antibodies that can be isolated: (i) the need for extensive screening to isolate even small numbers of high-affinity antibodies and (ii) immune responses against these antibodies when injected into humans. Thus, regardless of the methodology used for screening/isolation of therapeutic monoclonal antibodies (mAbs), the translation rate from discovery to clinic is inefficient and laborious.

One advance that accelerated the approval of therapeutic mAbs was the generation of humanized antibodies by the complementary-determining region (CDR) grafting technique. In CDR grafting, non-human antibody CDR sequences are transplanted into a human framework sequence to maintain target specificity.

### Humanized Antibody and Antibody-Drug Conjugate (ADC) Pharmaceutical Compositions

Cancer cells overexpressing HERV-K may be good targets for the anti-HERV-K humanized antibodies and antibody-drug conjugates of the invention, because more antibodies may be bound per cell. Thus, in a twenty-fifth embodiment, a cancer patient to be treated with anti-HERV-K humanized antibodies or antibody-drug conjugates of the invention is a patient, e.g., a breast cancer, ovarian cancer, pancreatic cancer, lung cancer or colorectal cancer patient who was diagnosed to have overexpression of HERV-K in their tumor cells.

Upon purifying anti-HERV-K humanized antibodies or antibody-drug conjugates they may be formulated into pharmaceutical compositions using well known pharmaceutical carriers or excipients.

The pharmaceutical compositions may be formulated with pharmaceutically acceptable carriers or diluents as well as any other known adjuvants and excipients under conventional techniques such as those disclosed in Remington: The Science and Practice of Pharmacy, 19th Edition, Gennaro, Ed. (Mack Publishing Co., Easton, Pa., 1995).

The pharmaceutically acceptable carriers or diluents as well as any other known adjuvants and excipients should be suitable for the humanized antibodies or antibody-drug conjugates of the invention and the chosen mode of administration. Suitability for carriers and other components of pharmaceutical compositions is determined based on the lack of significant negative impact on the desired biological properties of the chosen compound or pharmaceutical composition of the invention, e.g., less than a substantial impact (10% or less relative inhibition, 5% or less relative inhibition, etc.) on antigen binding.

A pharmaceutical composition of the invention may also include diluents, fillers, salts, buffers, detergents (e.g., a nonionic detergent, such as Tween-20 or Tween-80), stabilizers (e.g., sugars or protein-free amino acids), preservatives, tissue fixatives, solubilizers, or other materials suitable to include in a pharmaceutical composition.

The actual dosage levels of the humanized antibodies or antibody-drug conjugates in the pharmaceutical compositions of the invention may be varied to obtain an amount of the humanized antibodies or antibody-drug conjugates effective to achieve the desired therapeutic response for a particular patient, composition, and mode of administration, without being toxic to the patient. The selected dosage level will depend upon a variety of pharmacokinetic factors including the activity of the particular compositions of the invention used, the route of administration, the time of administration, the rate of excretion of the particular compound being used, the duration of the treatment, other drugs, compounds and/or materials used combined with the particular compositions used, the age, sex, weight, condition, general health and prior medical history of the patient being treated, and like factors well known in the medical arts.

The pharmaceutical composition may be administered by any suitable route and mode. Suitable routes of administering the humanized antibodies or antibody-drug conjugates of the invention are well known in the art and may be selected by those of ordinary skill in the molecular biological art.

In a twenty-sixth embodiment, the pharmaceutical composition of the invention is administered parenterally.

The phrases parenteral administration and administered parenterally means modes of administration other than enteral and topical administration, usually by injection, and include epidermal, intravenous, intramuscular, intraarterial, intrathecal, intracapsular, intraorbital, intracardiac, intradermal, intraperitoneal, intratendinous, transtracheal, subcutaneous, subcuticular, intraarticular, subcapsular, subarachnoid, intraspinal, intracranial, intrathoracic, epidural and intrasternal injection and infusion.

In a twenty-seventh embodiment, the pharmaceutical composition is administered by intravenous or subcutaneous injection or infusion.

Pharmaceutically acceptable carriers include any suitable solvents, dispersion media, coatings, antibacterial and antifungal agents, isotonicity agents, antioxidants and absorption delaying agents, and the like that are physiologically compatible with humanized antibodies or antibody-drug conjugates of the invention.

Examples of suitable aqueous and nonaqueous carriers which may be used in the pharmaceutical compositions of the invention include water, saline, phosphate buffered saline, ethanol, dextrose, polyols (such as glycerol, propylene glycol, polyethylene glycol, and the like), and suitable mixtures thereof, vegetable oils, such as olive oil, corn oil, peanut oil, cottonseed oil, and sesame oil, carboxymethyl cellulose colloidal solutions, tragacanth gum and injectable organic esters, such as ethyl oleate, and/or various buffers. Other carriers are well known in the pharmaceutical arts.

Pharmaceutically acceptable carriers include sterile aqueous solutions or dispersions and sterile powders for preparing sterile injectable solutions or dispersion. Using such media and agents for pharmaceutically active substances is known in the art. Except as far as any conventional media or agent is incompatible with the anti-HERV-K humanized antibodies or antibody-drug conjugates of the invention, use thereof in the pharmaceutical compositions of the invention is contemplated.

Proper fluidity may be maintained, for example, using coating materials, such as lecithin, by the maintenance of the required particle size for dispersions, and using surfactants.

The pharmaceutical compositions of the invention may also comprise pharmaceutically acceptable antioxidants for instance (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

The pharmaceutical compositions of the invention may also comprise isotonicity agents, such as sugars, polyalcohols such as mannitol, sorbitol, and glycerol, or sodium chloride in the compositions.

The pharmaceutical compositions of the invention may also contain one or more adjuvants for the chosen route of administration such as preservatives, wetting agents, emulsifying agents, dispersing agents, preservatives, or buffers, which may enhance the shelf life or effectiveness of the pharmaceutical composition. The anti-HERV-K humanized antibodies or antibody-drug conjugates of the invention may be prepared with carriers that will protect the compound against rapid release, such as a controlled release formulation, including implants, transdermal patches, and microencapsulated delivery systems. Such carriers may include gelatin, glyceryl monostearate, glyceryl distearate, biodegradable, biocompatible polymers such as ethylene vinyl acetate, polyanhydrides, polyglycolic acid, collagen, polyorthoesters, and polylactic acid alone or with a wax, or other materials well known in the molecular biological art. Methods for preparing such formulations are generally known to those skilled in the molecular biological art. See e.g., Sustained and Controlled Release Drug Delivery Systems, Robinson, ed. (Marcel Dekker, Inc., New York, 1978).

In a twenty-eighth embodiment, the anti-HERV-K humanized antibodies or antibody-drug conjugates of the invention may be formulated to ensure proper distribution in vivo. Pharmaceutically acceptable carriers for parenteral administration include sterile aqueous solutions or dispersions and sterile powders for preparing sterile injectable solutions or dispersion. Using such media and agents for pharmaceutically active substances is known in the art. Except as far as any conventional media or agent is incompatible with the active compound, use thereof in the pharmaceutical compositions of the invention is contemplated. Supplementary active compounds may also be incorporated into the compositions.

Pharmaceutical compositions for injection must typically be sterile and stable under the conditions of manufacture and storage. The composition may be formulated as a solution, micro-emulsion, liposome, or other ordered structure suitable to high drug concentration. The carrier may be an aqueous or nonaqueous solvent or dispersion medium containing for instance water, ethanol, polyols (such as glycerol, propylene glycol, polyethylene glycol, and the like), and suitable mixtures thereof, vegetable oils, such as olive oil, and injectable organic esters, such as ethyl oleate. The proper fluidity may be maintained, for example, using a coating such as lecithin, by the maintenance of the required particle size for dispersion and using surfactants. Often, it will be preferable to include isotonic agents, for example, sugars, polyalcohols such as glycerol, mannitol, sorbitol, or sodium chloride in the composition. Prolonged absorption of the injectable compositions may be brought about by including in the composition an agent that delays absorption, for example, monostearate salts and gelatin. Sterile injectable solutions may be prepared by incorporating the anti-HERV-K humanized antibodies or antibody-drug conjugates in the required amount in an appropriate solvent with one or a combination of ingredients, e.g., as enumerated above, as required, followed by sterilization microfiltration. Generally, dispersions are prepared by incorporating the anti-HERV-K humanized antibodies or antibody-drug conjugates into a sterile vehicle that contains a basic dispersion medium and the required other ingredients e.g., from those enumerated above. For sterile powders for preparing sterile injectable solutions, examples of methods of preparation are vacuum drying and freeze-drying (lyophilization) that yield a powder of the active ingredient plus any additional desired ingredient from a previously sterile-filtered solution thereof.

Sterile injectable solutions may be prepared by incorporating the anti-HERV-K humanized antibodies or antibody-drug conjugates in the required amount in an appropriate solvent with one or a combination of ingredients enumerated above, as required, followed by sterilization microfiltration. Generally, dispersions are prepared by incorporating the anti-HERV-K humanized antibodies or antibody-drug conjugates into a sterile vehicle that contains a basic dispersion medium and the required other ingredients from those enumerated above. For sterile powders for preparing sterile injectable solutions, examples of methods of preparation are vacuum drying and freeze-drying (lyophilization) that yield a powder of the anti-HERV-K humanized antibodies or antibody-drug conjugates plus any additional desired ingredient from a previously sterile-filtered solution thereof.

The pharmaceutical composition of the invention may contain one anti-HERV-K humanized antibodies or antibody-drug conjugates of the invention or a combination of anti-HERV-K humanized antibodies or antibody-drug conjugates of the invention.

The efficient dosages and the dosage regimens for the anti-HERV-K humanized antibodies or antibody-drug conjugates depend on the disease or condition to be treated and may be determined by the persons skilled in the molecular biological art. An exemplary, non-limiting range for a therapeutically effective amount of a compound of the invention is about 0.1-100 mg/kg. An exemplary, non-limiting range for a therapeutically effective amount of an anti-HERV-K humanized antibodies or antibody-drug conjugates of the invention is about 0.02-30 mg/kg, in particular of the antibodies 011, 098, 114 or 111 as disclosed herein. Further guidance regarding the therapeutically effective amount is provided by Hendrikx et al., Fixed dosing of monoclonal antibodies in oncology. Oncologist, 22(10), 1212-1221 (October 2017) and Lu et al., Development of therapeutic antibodies to treat diseases. Journal of Biomedical Science, volume 27, Article number 1 (Jan. 2, 2020).

A physician having ordinary skill in the medical art may readily determine and prescribe the effective amount of the pharmaceutical composition required. For example, the physician could start doses of the anti-HERV-K humanized antibodies or antibody-drug conjugates used in the pharmaceutical composition at levels lower than that required to achieve the desired therapeutic effect and gradually increase the dosage until the desired effect is achieved. A suitable daily dose of a composition of the invention will be that amount of the compound which is the lowest dose effective to produce a therapeutic effect. Such an effective dose will generally depend upon the factors described above. Administration can be intravenous, intramuscular, intraperitoneal, or subcutaneous, and for instance administered proximal to the site of the target. If desired, the effective daily dose of a pharmaceutical composition may be administered as two, three, four, five, six or more sub-doses administered separately at intervals throughout the day, optionally, in unit dosage forms. While it is possible for an anti-HERV-K humanized antibodies or antibody-drug conjugates of the invention to be administered alone, it is preferable to administer the anti-HERV-K humanized antibodies or antibody-drug conjugates as a pharmaceutical composition as described above.

In a twenty-ninth embodiment, the anti-HERV-K humanized antibodies or antibody-drug conjugates may be administered by infusion in a weekly dosage of from 10 to 1500 mg/m², such as from 30 to 1500 mg/m², or such as from 50 to 1000 mg/m², or such as from 10 to 500 mg/m², or such as from 100 to 300 mg/m². Such administration may be repeated, e.g., one time to eight times, such as three times to five times. The administration may be performed by continuous infusion over a period of from two hours to twenty-four hours, such as from two hours to twelve hours.

In a thirtieth embodiment, the anti-HERV-K humanized antibodies or antibody-drug conjugates may be administered by infusion every third week in a dosage of from 30 to 1500 mg/m², such as from 50 to 1000 mg/m² or 100 to 300 mg/m². Such administration may be repeated, e.g., one time to eight times, such as three times to five times. The administration may be performed by continuous infusion over a period of from two hours to twenty-four hours, such as from two hours to twelve hours.

In a thirty-first embodiment, the anti-HERV-K humanized antibodies or antibody-drug conjugates may be administered by slow continuous infusion over a prolonged period, such as more than twenty-four hours, to reduce toxic side effects.

In a thirty-second embodiment the anti-HERV-K humanized antibodies or antibody-drug conjugates may be administered in a weekly dosage of 50 mg to 2000 mg, for up to sixteen times, The administration may be performed by continuous infusion over a period from two to twenty-four hours. Such regimen may be repeated one or more times for example, after six months or twelve months. The dosage may be determined or adjusted by measuring the anti-HERV-K humanized antibodies or antibody-drug conjugates of the invention in the blood upon administration, by for instance taking out a biological sample and using anti-idiotypic antibodies which target the antigen binding region of the anti-HERV-K humanized antibodies or antibody-drug conjugates of the invention. Further guidance regarding the dosage is provided by Hendrikx et al., Fixed dosing of monoclonal antibodies in oncology. Oncologist, 22(10), 1212-1221 (October 2017) and Lu et al., Development of therapeutic antibodies to treat diseases. Journal of Biomedical Science, volume 27, Article number 1 (Jan. 2, 2020).

In a thirty-third embodiment, the anti-HERV-K humanized antibodies or antibody-drug conjugates may be administered by maintenance therapy, such as, e.g., once a week for six months or more.

In a thirty-fourth embodiment, the antibody-drug conjugate may be administered by a regimen including one infusion of an antibody-drug conjugate of the invention followed by an infusion of an anti-HERV-K antibody of the invention, such as antibody 6H5hum.

### Bispecific T Cell Engagers (BiTEs)

In a thirty-fifth embodiment, provided herein is a method of treating a HERV-K-positive cancer in a subject in need thereof, comprising administering to the subject a therapeutically effective amount of a bispecific antibody comprising two antigen-binding regions, one which has a binding specificity for CD3 or CD8 and one which has a binding specificity for HERV-K.

In a thirty-sixth embodiment, the invention relates to a bispecific antibody comprising a first single chain human variable region which binds to HERV-K, in series with a second single chain human variable region which binds to T cell activation ligand CD3 or CD8. T the first and second single chain human variable regions are in amino to carboxy order, wherein a linker sequence intervenes between each segment, and wherein a spacer polypeptide links the first and second single chain variable regions.

In a thirty-seventh embodiment of the method, the administering is intravenous or intraperitoneal.

In a thirty-eighth embodiment of the method, the bispecific binding molecule is not bound to a T cell during said administering step.

In a thirty-ninth embodiment, the method further comprises administering T cells to the subject. In a specific embodiment, the T cells are bound to molecules identical to said bispecific binding molecule.

In a fortieth embodiment, provided herein is a pharmaceutical composition comprising a therapeutically effective amount of the bispecific binding molecule, a pharmaceutically acceptable carrier, and T cells. In a forty-first embodiment, the T cells are bound to the bispecific binding molecule. In a forty-second embodiment, the binding of the T cells to the bispecific binding molecule is noncovalently. In a forty-third embodiment, the administering is performed combined with T cell infusion to a subject for treatment of a HERV-K-positive cancer. In a forty-fourth embodiment, the administering is performed after treating the patient with T cell infusion. In a forty-fifth embodiment, the T cells are autologous to the subject to whom they are administered. In a forty-sixth embodiment, the T cells are allogeneic to the subject to whom they are administered. In a forty-seventh embodiment, the T cells are human T cells.

In a forty-eighth embodiment, the subject is a human.

In a forty-ninth embodiment of the method, the bispecific binding molecule is in a pharmaceutical composition, which pharmaceutical composition further comprises a pharmaceutically acceptable carrier.

In a fiftieth embodiment of the bispecific binding molecule, the bispecific binding molecule does not bind an Fc receptor in its soluble or cell-bound form. In some embodiments of the bispecific binding molecule, the heavy chain was mutated to destroy an N-linked glycosylation site. In a fifty-first embodiment of the bispecific binding molecule, the heavy chain has an amino acid substitution to replace an asparagine that is an N-linked glycosylation site, with an amino acid that does not function as a glycosylation site. In a fifty-second embodiment of the bispecific binding molecule, the heavy chain was mutated to destroy a C1q binding site. In a fifty-third embodiment, the bispecific binding molecule does not activate complement.

In a fifty-fourth embodiment of the bispecific binding molecule, the HERV-K-positive cancer is breast cancer, ovarian cancer, prostate cancer, pancreatic cancer, melanoma, colorectal cancer, small cell lung cancer, non-small cell lung cancer or any other neoplastic tissue that expresses HERV-K. In a fifty-fifth embodiment, the HERV-K-positive cancer is a primary tumor or a metastatic tumor, e.g., brain, bone, or lung metastases.

### DNA-Encoded Bi-Specific T Cell Engagers (DBiTE)

Specific antibody therapy, including mAbs and bispecific T cell engagers (BiTEs), are important tools for cancer immunotherapy. BiTEs are a class of artificial bi-specific monoclonal antibodies with the potential to transform the immunotherapy landscape for cancer. BiTEs direct a host's immune system, more specifically the T cells' cytotoxic activity, against cancer cells. BiTEs have two binding domains. One domain binds to the targeted tumor (like HERV-K-expressing cells) while the other engages the immune system by binding directly to molecules on T cells. This double-binding activity drives T cell activation directly at the tumor resulting in a killing function and tumor destruction. DBiTEs share many advantages of bi-specific monoclonal antibodies. Both comprise engineered DNA sequences which encode two antibody fragments. The patient's own cells become the factory to manufacture functional BiTES encoded by the delivered DBiTE sequences. Delivery of BiTEs and permitting combinations of DBiTEs to be administered at one time as a multi-pronged approach to treat resistant cancer. Synthetic DNA designs for BiTE-like molecules include engineering and encoding them in optimized synthetic plasmid DNA cassettes. DBiTEs are then injected locally into the muscle and muscle cells convert the genetic instructions into protein to allow for direct in vivo launching of the molecule directly into the bloodstream to the seek and destroy tumors. See, Perales-Puchalt et al., JCI Insight, 4(8), e126086 (Apr. 18, 2019). In preclinical studies, DBiTEs demonstrated a unique profile compared to conventional BiTEs, overcoming some of the technical challenges associated with production. For further information, see also, PCT Patent Publications WO 2016/054153 (The Wistar Institute of Anatomy and Biology) and WO 2018/041827 (Psioxus Therapeutics Limited).

### HERV-K CAR-T Therapies

Many formulations of CARs specific for target antigens have been developed. See e.g., International Pat. Publ. WO 2014/186469 (Board of Regents, the University of Texas System). This specification provides a method of generating chimeric antigen receptor (CAR)-modified T cells with long-lived in vivo potential to treat, for example, leukemia patients exhibiting minimal residual disease (MRD). In aggregate, this method describes how soluble molecules such as cytokines can be fused to the cell surface to augment therapeutic potential. The core of this method relies on co-modifying CAR T cells with a human cytokine mutein of interleukin-15 (IL-15), henceforth called mIL15. The mIL15 fusion protein comprises codon-optimized cDNA sequence of IL-15 fused to the full length IL15 receptor alpha via a flexible serine-glycine linker. This IL-15 mutein was designed in such a fashion to: (i) restrict the mIL15 expression to the surface of the CAR+ T cells to limit diffusion of the cytokine to non-target in vivo environments, potentially improving its safety profile as exogenous soluble cytokine administration has led to toxicities; and (ii) present IL-15 in the context of IL-15Ra to mimic physiologically relevant and qualitative signaling as well as stabilization and recycling of the IL15/IL15Ra complex for a longer cytokine half-life. T cells expressing mIL15 are capable of continued supportive cytokine signaling, which is critical to their survival post-infusion. The mIL15+CAR+ T cells generated by non-viral Sleeping Beauty System genetic modification and subsequent ex vivo expansion on a clinically applicable platform yielded a T cell infusion product with enhanced persistence after infusion in murine models with high, low, or no tumor burden. The mIL15 CAR+ T cells also demonstrated improved anti-tumor efficacy in both the high and low tumor burden models. A hu6H5 scFv was used to generate a K-CAR in a lentiviral vector.

### Combination Therapies

The therapies of this specification can be used without modification, relying on the binding of the antibodies or fragments to the surface antigens of HERV-K+ cancer cells in situ to stimulate an immune attack thereon. The method can be carried out using the antibodies or binding fragments to which a cytotoxic agent is bound. Binding of the cytotoxic antibodies, or antibody binding fragments, to the tumor cells inhibits the growth of or kills the cells.

Antibodies specific for HERV-K env protein may be used with other expressed HERV antigens. This may be useful for immunotherapy and antibody treatments of diseases in which several different HERVs are expressed. For example, HERV-E in prostate, ERV3, HERV-E and HERV-K in ovarian cancer, and ERV3, HERV-H, and HERV-W in other cancers.

Cytokines in the common gamma chain receptor family (γC) are important costimulatory molecules for T cells critical to lymphoid function, survival, and proliferation. IL-15 possesses several attributes desirable for adoptive therapy. IL-15 is a homeostatic cytokine that supports the survival of long-lived memory cytotoxic T cells, promotes the eradication of established tumors via alleviating functional suppression of tumor-resident cells, and inhibits activation-induced cell death (AICD). IL-15 is tissue restricted and only under pathologic conditions is it observed at any level in the serum, or systemically. Unlike other γC cytokines secreted into the surrounding milieu, IL-15 is trans-presented by the producing cell to T cells in the context of IL-15 receptor alpha (IL-15Ra). The unique delivery mechanism of this cytokine to T cells and other responding cells: (i) is highly targeted and localized, (ii) increases the stability and half-life of IL-15, and (iii) yields qualitatively different signaling than is achieved by soluble IL-15.

### Pharmaceutical Compositions

This specification is also directed to pharmaceutical compositions comprising a therapy that specifically binds to a HERV-K env protein, with a pharmaceutically acceptable carrier, excipient, or diluent. Such pharmaceutical compositions may be administered in any suitable manner, including parental, topical, oral, or local (such as aerosol or transdermal) or any combination thereof. Suitable regimens also include an initial administration by intravenous bolus injection followed by repeated doses at one or more intervals.

Pharmaceutical compositions of the compounds of the disclosure are prepared for storage by mixing a peptide ligand containing compound having desired purity with optional pharmaceutically acceptable carriers, excipients, or stabilizers (Remington's Pharmaceutical Sciences 18th ed., 1990), in lyophilized formulations or aqueous solutions. Acceptable carriers, excipients, or stabilizers are nontoxic to recipients at the dosages and concentrations used.

The compositions herein may also contain more than one active compound for the particular indication being treated, preferably those with complementary activities that do not hurt each other. Or in addition, the composition may comprise a cytotoxic agent, cytokine, growth inhibitory agent, or cardioprotectant. Such molecules are suitably present in combination in amounts effective for the purpose.

The invention is further illustrated by the following EXAMPLES, which are not intended to limit the scope or content of the invention in any way.

### Example 1

### Humanized Antibodies Produced Commercially and in the Inventors' Laboratories

The inventors synthesized and purified humanized antibodies targeting HERV-K tumor antigens. HERV-K envelope surface gene tumor antigens (KSU) were derived from human patient cancer cells, rather sequences in GenBank, which are HERV-K gene sequences from normal humans. These HERV-K sequences from cancer patients were shown by the inventors to contain variants that differentiated them from the normal HERV-K sequence. The inventors' humanized antibodies are specific for the HERV-K target found in human cancer cells. This specificity distinguishes the HERV-K target found in human cancer cells from the HERV-K target present in tissues from normal individuals or patients with non-cancer disorders. This specificity also distinguishes the inventors' humanized antibodies to HERV-K tumor antigens from other antibodies to HERV-K tumor antigens.

The human antibodies targeted the full-length surface protein of the HERV-K envelope gene, rather than a peptide or a small fragment of the gene. Full length envelope surface protein is only expressed in cancer cells obtained from cancer patients.

The scFv sequence from a murine antibody against HERV-K envelope surface fusion protein was submitted to a contract research organization (CRO) to produce humanized antibodies. The first CRO could not generate the light chain of the inventors' humanized antibody targeting HERV-K, showing the unexpectedness of producing a HER-K antagonist humanized antibody that includes the heavy chain and light chain.

A second contract research organization generated a chimeric antibody, but that antibody could not bind the HERV-K envelope surface fusion protein by ELISA, SPR, or Western blot assay, even though binding of the chimeric antibody and the inventors' non-humanized mouse antibody was readily detected using ELISA or Western blot assays. This result also shows the unexpectedness of producing an active HER-K antagonist humanized antibody that includes the heavy chain and light chain.

The inventors then generated three humanized antibodies. Only one of the three humanized antibodies (HUM1, expressed in bacterial cells) bound HERV-K antigen. Expressed HUM2 or HUM3 protein did not bind the recombinant SU protein well, and especially not to the SU proteins produced from breast cancer cells (MDA-MB-231).

The inventors then produced an additional humanized antibody, hu6H5. Hu6H5 was expressed in mammalian cells and its antitumor effects were determined. Both the HUM1 and hu6H5 antibodies bound to the full-length KSU antigen.

The difference between HUM1, HUM2, and HUM3 is shown in TABLE 1 for VH below and TABLE 2 for VL below. HUM1, HUM2, and HUM3 were all generated from the same bacterial expression vector. All have the same CDRs for VH and VL. Hu6H5 was generated from a mammalian vector based on the HUM1 sequence. HUM1, HUM2, and HUM3 all bound recombinant KSU protein. Only HUM1 bound the protein isolated from cancer cells.

This EXAMPLE unexpectedly shows that the medically useful functional property of antibody binding to protein isolated from cancer cells is not a property arising from the structure of VH and VL CDRs.

### Example 2

Comparison of hu6H5 Sequence with Sequences of Other Humanized Anti-HERV-K Antibodies, and with Sequences of Other Antibodies that Target Tumor Antigens

An anti-HERV-K antibody that targets amyotrophic lateral sclerosis (ALS), called GN-mAb-Env_K01, binds to an HERV-K envelope linear peptide with the SLDKHKHKKLQSFYP core sequence. See U.S. Pat. No. 10,723,787.

The hu6H5 antibody binds to the longer, full-length HERV-K envelope SU domain, and not a linear peptide.

In contrast to GN-mAb-Env_K01, the inventors' humanized antibody was specific for HERV-K ENV protein from a cancer cell target. The hu6H5 sequence shows little sequence similarity to the GN-mAb-Env_K01 humanized antibody. The identities are 60% for VH and 55% for VL.

A BLAST search showed that other antibodies that target cancer-relevant antigens have a minimal amount of homology with hu6H5 (6-15 peptides). These include 82% identity with the VH of anti-ErbB2 antibody (based on the crystal structure of the anti-ErbB2 Fab2C4) that targets HER2-positive breast cancer, and 88% identity with the VL of anti-DREG-55 [(anti-DREG-55 (L-selectin) immunoglobulin light chain variable region]. The anti-DREG-55 target, L-selectin, mediates adaptive and innate immunity in cancer.

The inventors also identified human germline sequences near the boundaries for CDRs in their humanized anti-HERV-K antibodies. These sequences include VRQAPGKGLEW (SEQ ID NO: 4). and LQMNSLRAEDTAVYYC (SEQ ID NO: 47).

### Example 3

### Binding Affinity of Anti-HERV-K Humanized Antibodies to HERV-K SU Protein, and Antibody Internalization

The inventors determined by ELISA assay that the HUM1 humanized antibody affinity toward the HERV-K envelope surface fusion protein was as effective as the affinity of their m6H5 at antibody concentrations above 0.00625 μg/ml and was more effective than most of inventors' other murine mAbs. See FIG. 2. The inventors' chimeric humanized anti-HERV-K antibody was shown by immunoblot to bind and their m6H5 mAb to two HERV-K Env surface proteins, ERVK6 and HERV-K envelope surface fusion protein.

To determine antibody internalization of humanized and murine antibodies, cells were incubated with HUM1 or m6H5 at 370° C. for different time intervals (0, 5, 30, and 45 minutes). At each time point, cells were fixed. Half of the cells were permeabilized and half were not permeabilized. Anti-human IgG 488 or anti-mouse IgG 488 was used to detect the percentages of antibody-bound HERV-K env protein that remained on the cell surface in the cells that were not permeabilized. A reduced surface expression of HERV-K positive cells was observed. The internalization of rate was determined in the cells that were permeabilized. The equation for the percentage of internalization at each point was 100—(% label at time 0).

Results demonstrated an increased expression of internalized HERV-K positive cells in cells treated with either HUM1 or murine 6H5 mAb, but HUM1 disappeared from the cell surface more rapidly than 6H5, indicating a more rapid uptake of the inventors' humanized antibody than their murine antibody. This rapid uptake supports the unique capability of HUM1 to deliver a payload more rapidly than m6H5.

### Example 4

### Cancer Cell Killing and Apoptosis

HERV-K transduction into 231K cells resulted in increased HERV-K expression in these cells. Flow cytometry results revealed that humanized (HUM1) and murine (6H5) anti-HERV-K antibodies bind to MDA-MB-231 cells, and that the inventors' therapeutic mAbs induce apoptosis more strongly in cells that express higher levels of HERV-K. Then, patterns of induction of early apoptosis and late apoptosis in BXPC 3 pancreatic cancer cells treated with hu6H5 and m6H5 were evaluated. Patterns of induction were similar for both humanized and mouse mAbs. However, hu6H5 treatment led to increases in both early and late apoptosis relative to m6H5 treatment, indicating the superiority of humanized mAb treatment over that of the murine mAb. Data from these studies indicate that the presence of increased levels of HERV-K antigen in or on breast cancer cells enables the inventors' humanized anti-HERV-K antibodies to target and kill those cells more effectively by inducing both early and late apoptosis.

### Example 5

Patterns of Breast Cancer Cell Signaling Pathway RNA Expression, with and without Induction of HERV-K Protein Overexpression

The effect of HERV-K overexpression in MDA-MB-231 breast cancer cells (231K) on RNA expression of cell signaling pathway genes, assessed by RNA sequencing (RNA-Seq), was compared with expression in 231C non-transduced cells. The inventors demonstrated that the PI3K-Akt and TNF signaling pathway were strongly upregulated (greater than 5-fold increase) in cells whose HERV-K expression was induced. In addition, MAPK, mTOR, and NF-κB and Ras and the Ras-associated protein Rap-1 showed a greater than 3-fold increase in 231K cells versus 231C cells.

A treatment group of immunodeficient mice (KC) were inoculated with 231K cells and compared with a control group of immunodeficient mice (C) inoculated with 231C cells. This in vivo cadre largely mirrored the results of the in vitro assay, showing major upregulation PI3K-Akt, TNF, MAPK, mTOR, and NF-κB, and more moderate upregulation of Ras and Rap-1 in tumors of the KC group of mice.

### Example 6

Patterns of Breast Cancer Cell Signaling Pathway Protein Expression in Response to Humanized and Murine mAb Treatment, with and without HERV-K Protein Overexpression

HERV-K was introduced into MDA-MB-231 human breast cancer cells by a retroviral vector, as described in EXAMPLE 4. The primary and secondary antibodies and their dilutions employed for the flow cytometric analysis of HERV-K and signal transduction pathway expression were:
m6H5: 1:200; anti-mouse: 1:1,000
Ras: 1:200; anti-rat: 1:1,000
p-ERK: 1:200; anti-mouse; 1:1,000
SIRT-1: 1:200; anti-rabbit: 1:1,000
CIDEA: 1:200; anti-mouse: 1:1,000
p-Rb: 1:200; anti-rabbit; 1:1,000
PE labelled Caspase 3: 1:200
Primary antibody incubation: one hour at 4° C.
Secondary antibody incubation: thirty minutes at 4° C.

The inventors observed reduced expression of HERV-K, Ras, p-ERK, and SIRT-1 in 231K cells treated with hu6H5, and the inhibitory effect of hu6H5 was much greater than was the effect of the murine m6H5 on these pathways. The documented positive effect of SIRT-1 expression on p53-mediated apoptosis and its negative regulation of p53-induced cellular senescence have led to the finding that inhibition of SIRT1 activity results in elevated p53 acetylation and transactivation and resultant enhanced apoptosis and inhibition of cell growth and multiplication. The change in expression observed for SIRT-1 after HERV-K antibody therapy has not been previously reported. Thus, hu6H5-induced inhibition of SIRT-1 expression would be beneficial for cancer treatment and additionally agrees on the inventors' previous findings that reduced HERV-K expression has a marked influence on the p53 signaling pathway. Likewise, 231K cells treated with hu6H5 showed a greater increase than 231C cells in the expression of pro-apoptotic Caspase 3 and the tumor suppressor gene Rb, further supporting the effectiveness of the inventors' humanized antibody in stimulating pathways that promote tumor cell death.

### Example 7

### Determination of Percentages of CD3 or CD8 BiTE Recombinant Proteins Using Anti-GST mAb and BiTE Binding to Target Cells

The percentage of peripheral blood mononuclear cells containing anti-HERV-K scFv linked to either anti-CD3 or anti-CD8 antibodies was determined by flow cytometry using an anti-GST antibody tag. The percentage for CD3 BiTEs ranged from 54.1% to 58.5%. The percentage for CD8 BiTEs ranged from 48.8% to 55.2%.

CD3BiTE/CD8BiTE binding to their respective targets was evaluated by ELISA. Wells were coated with CD3 epsilon or CD8 protein (1 μg/ml) followed by addition of increasing concentrations of BiTE. Mouse anti-GST and subsequently goat anti-mouse IgG-HRP (or Isotype) were added, followed by ABTS for color development. The ELISA results demonstrate that CD3BiTE bound to CD3 epsilon, but CD3BiTE also cross-reacted with CD8 protein. CD8BiTE bound CD8 protein but did not cross-react with CD3 epsilon.

### Example 8

### Effect of BiTE Treatment on Killing of Cancer Cells

Cell killing with either BiTE was robust. Both CD3 and CD8 BiTE treatment resulted in increased cell killing of human breast (MDA-MB-231, MCF7), pancreatic (BxPC3), and murine ovarian (ID8) cancer cells in vitro. Notably, in LDH assays the mean fluorescence intensity increased from 125 to nearly 3,500 when MDA-MB-231 cells were treated with either CD3 or CD8 BiTEs, and similar although less dramatic increases in cell killing were observed for the other cell lines. Direct microscopic observation of MDA-MB-231 cells treated with HERV-K CD3 BiTE at levels of 0, 0.03, 0.3, 30, and 300 ng/ml of BiTE also revealed dose-response and time effects of HERV-K targeted CD3 BiTE on killing of MDA-MB-231 cells, with higher BiTE concentrations and longer treatment times leading to increased cell death. However, cell death was not increased when cancer cells were treated with BiTEs only, without the presence of added peripheral blood mononuclear cells or T cells. The strong increase in cell killing in the presence of T cells supports the in vitro efficacy of our HERV-K-targeting BiTEs.

### Example 9

Design of Humanized Single Chain Variable Fragment (scFv) Antibody

Antibody numbering scheme and CDR definitions: The antibody-numbering server is part of KabatMan database and was used to number all antibody sequences of this EXAMPLE according to the enhanced Chothia scheme. In this humanization EXAMPLE, the inventors combined the Enhanced Chothia numbering with the Contact CDR definition of antibody sequence to position the CDRs of antibody light chain and heavy chains at these locations: H-CDR1 30-35, H-CDR2 47-58 H-CDR3 93-101, L-CDR1 30-36, L-CDR2 46-55, and L-CDR3 89-96.

Selection of the human template: To generate humanized scFv gene, six Complementary determine regions (CDRs) of mouse VH and VL were grafted onto selected human Frameworks (FRs) showing the highest amino acids sequence identify to the humanization of the antibodies. The human immunoglobulin germline sequences were used as the selected human FRs for mouse FWJ antibody clone. Human immunoglobulin germline sequences showing the highest amino acid sequences similarity in FRs between human and mouse FWJ VH and VL were identified independently using from V-quest server (http://www.imgt.org/IMGT_vquest) and Ig-BLAST server (http://www.ncbi.nlm.nih.gov/igblast). The selected heavy chain VHIII and the light KI chain based conserved germlines. The consensus human FRs was designed among selected germline gene for grafting CDRs residues of FWJ. The amino acid sequences in FRs of mouse VH and VL that differed from consensus human FRs were substituted with human residues, while preserving mouse residues at position known as Vernier zone residues and chain packing residues.

TABLE 1
VHs from HUM1, HUM2, and MuVH

| | |
|---|---|
| VH CDRs | SCKASGYSFTGYYMHWV000IGRVNPNSGGTSYNQKFKD---CARSKGNYFYAHDY |
| Hum2_VH | EVQLVESGGGLVQPGGSLKVSCKASGYSFTGYYMHWVRQASGKGLEWIGRVNPNSGGTSY |
| HUM1_VH | EVQLVESGGGLVQPGGSLRLSCKASGYSFTGYYMHWVRQAPGKGLEWIGRVNPNSGGTSY |
| FWJ_MUVH | QVKLQQSGPDLVKPGASVKISCKASGYSFTGYYMHWVKQSHGKSLEWIGRVNPNSGGTSY |
| | :*:*:** .**:**.*:::*****************:*: **.**************** |
| Hum2_VH | NQKFKDRFTISRDKSISTLYLQMSSLRSEDTAVYYCARSKGNYFYAMDYWGQGTLVTVSS |
| HUM1_VH | NQKFKDRATLSVDNSKNTAYLQMNSLRAEDTAVYYCARSKGNYFYAMDYWGQGTLVTVSS |
| FWJ_MUVH | NQKFKDKAILTVDKSSSTAYMELRSLTSEDSAVYYCARSKGNYFYAMDYWGQGTTVTVSS |
| | ******::: *:* .* *::: **:**:*********************** ***** |
| Hum2_VH | (SEQ ID NO: 7) |
| HUM1_VH | (SEQ ID NO: 6) |
| FWJ_MUVH | (SEQ ID NO: 8) |

“*” means that the residues or nucleotides in that column are identical in all sequences in the alignment.
“:” means that conserved substitutions are observed.
“.” means that semi-conserved substitutions are observed, i.e., amino acids having similar shape.

TABLE 2
VLs form HUM2, HUM3, and MuVL compared

| | |
|---|---|
| VL CDRs | CRASESVDSHGTSFMR---RASNLE---QQSNEDPPT |
| HUM2_VL | DIQMTQSPSSLSASVGDRVTITCRASESVDSHGTSFMHWYQQKPGKAPKFLIYRASMLES |
| HUM3_VL | DIQMTQSPSSLSASVGDRVTISCRASESVDSHGTSFMHWYQQKPGKSPKFLIYRASNLES |
| FWJ_MUVL | DIELTQSPASLAVSLGQRATISCRASESVDSHGTSFMHWYQQKPGQPPKFLIYRASNLES |
| | **::****:**:.*:*:*.**:************************.************* |
| HUM2_VL | GIPSRFSGSGSGTDFTLTISSVQPEDFAVYYCQQSNEDPPTFGGGTKVEIK |
| HUM3_VL | GIPSRFSGSGSGTDFTLTISSLQPEDFAIYYCQQSNEDPPTFGGGTKVEIK |
| FWJ_MUVL | GIPARFSGSGSRTDFTLTINPVETDDVAIYYCQQSNEDPPTFGGGTKLEIK |
| | ***:***.*** *******..::.:*.*:******************:*** |
| HUM2_VL | (SEQ ID NO: 9) |
| HUM3_VL | (SEQ ID NO: 10) |
| FWJ_MUVL | (SEQ ID NO: 11) |

As highlighted in yellow background in TABLE 2, the CDRs for both the mouse and the human CDRs are identical, but major differences in the remainder of the VL sequences may account in part for the inability of HUM2 and HUM3 to bind to the human cancer cell HERV-K envelope protein.

Construction of scFv and test of biological activity against Human KV and 231 antigens. The clone of variable heavy chain and light chain of FWJ_1 and FWJ_2 antibody gene were amplified and synthesized. The gene encoding the scFv is VH-linker-VL with a standard twenty amino acid linker (Gly₄Ser)₃GGGAR (SEQ ID NO: 14). The amplified gene was digested with BssHII and Nhel restriction enzymes and insert into a pET-based vector (PAB-myc) containing a pelB promotor for controlling periplasmic protein expression (Novagen, Madison, WI, USA) along with 6×histidine tag at the C-termini for purification by metal affinity chromatography and transformed into DH5α bacterial strain. The transformed clones were amplified in Luria Broth with ampicillin broth overnight. The plasmid DNAs were prepared and sent for DNA sequencing. The correct sequence of scFv plasmid was transformed into the T7 Shuffle bacterial strain and the transformed bacteria were used for soluble protein production in periplasmic compartment.

FWJ_1 and FWJ_2 scFv Gene and Translated Protein Sequences: The SEQUENCE LISTING delineates the heavy and light chains and linker arm of FWJ_1 and FWJ_2_scFv. In the engineering of the FWJ_1 and FWJ_2_scFv gene two epitope tags were engineered onto the carboxyl terminus: (1) a six His tag to facilitate purification of the encoded scFv by nickel affinity chromatography; and (2) a myc tag to facilitate rapid immunochemical recognition of the expressed scFv.

Induction of ScFv proteins in a bacterial host: The FWJ_1 and FWJ_2scFv clone were transformed into T7 shuffle bacterial strain. T7 shuffle cells and was grown in 1.4 L 2xYT plus ampicillin medium at 37° C. until log-phage (OD600=0.5), induced with 0.3 mM IPTG, and allowed to grow at 30° C. for an additional sixteen hours. After induction, the bacteria were harvested by centrifugation at 8000 g for fifteen minutes at 4° C., and the pellets were stored in −20° C. for at least two hours. The frozen pellets were briefly thawed and suspended in 40 ml of lysis buffer (1 mg/ml lysozyme in phosphate-buffered saline plus EDTA-free protease inhibitor cocktail (Thermo Scientific, Waltham, MA, USA). The lysis mixture was incubated on ice for an hour, and then 10 mM MgCl2 and 1 μg/ml DNase I were added, and the mixture was incubated at 25° C. for twenty minutes. The final lysis mixture was centrifuged at 12000 g for twenty minutes and the supernatants were collected. This supernatant was termed the periplasmic extract used for nickel column affinity chromatography.

Western blot analysis using FWJ 1 and FWJ 2 scFv protein: Lysate Ag and HERV-K envelope surface fusion protein were used as antigens target in dot-blot analyses. 2-5 μg Ag proteins as non-reduced conditional and 1 μg purified protein as negative control were loaded onto nitrocellulose membranes. The membrane was blocked using 3% skimmed milk in phosphate-buffered saline for three hours at room temperature. After that, the membrane was incubated with periplasmic extract of FWJ_1 and FWJ_2 scFv proteins overnight at 4° C. The membrane was washed with sodium phosphate-buffered saline with 0.05% Tween 20 buffer (PBST) three times. The washed membrane was incubated with anti-c Myc mouse IgG for one hour at room temperature to recognize the c-Myc tag on the scFv and identify the position of antigens bound by the scFv. After washing with PBST, the membrane was incubated with the goat anti-mouse IgG (H+L) HRP conjugate diluted (1:3000 v/v) in phosphate-buffered saline for one hour at room temperature, and specific immunoreactive bands were visualized with a mixture of TMB substrate.

The inventors identified anti-HERV-K mAb 6H5 heavy chain CDRs (H-CDR1 30-35, H-CDR2 47-58, H-CDR3 93-101), and light chain CDRs (L-CDR1 30-36, L-CDR2 46-55, and L-CDR3 89-96) and grafted them onto selected human frameworks (FRs) showing the highest amino acid sequence identity to optimize the humanization of the antibodies. Human immunoglobulin germline sequences showing the highest amino acid sequence similarity in FRs between human and mouse VH and VL were identified independently from the V-quest (http://www.imgt.org/IMGT_vquest) and Ig-BLAST (http://www.ncbi.nlm.nih.gov/igblast) servers. The amino acid sequences in FRs of mouse VH and VL that differ from consensus human FRs were substituted with human residues, while preserving mouse residues at positions known as Vernier zone residues and chain packing residues. The clone of VH and VL chains of candidate humanized antibody genes were amplified and synthesized. The gene encoding the scFv, which includes a VH-linker-VL with a standard twenty amino acid linker (Gly₄Ser)₃GGGAR (SEQ ID NO: 14), was inserted into a pET based vector (PAB-myc) containing a pelB promotor for controlling periplasmic protein expression (Novagen, Madison, WI, USA) along with a 6× histidine tag at the C-termini for purification by metal affinity chromatography and a myc tag to facilitate rapid immunochemical recognition of the expressed scFv. The correct sequences of the scFv plasmid were used for soluble protein production in the periplasmic compartment. Two hu6H5 clones (FWJ1 and FWJ2) were selected and binding affinities to antigen were determined. Both clones bound antigens produced from recombinant HERV-K Env surface fusion protein (KSU) and lysates from MDA-MB-231 breast cancer cells.

HuVH or HuVL with human IgG1 was cloned into a pcDNA 3.4 vector to produce VH-CH (human IgG1) or VL-CL (human Kappa). The plasmids were transiently transfected into Expi293 cells for mammalian expression. The ratio of H chain vs. L chain plasmids is 2:3. A Western Blot was used to determine expression, and the predicted MW of 49/23 kDa (heavy chain/light chain) under reducing conditions was detected. A Western blot was used to detect the VH chain and VL chain of the humanized anti-HERV-K antibody in an SDS-PAGE gel under reducing conditions. A 49 KDa molecular mass for the VH chain and a 23 KDa molecular mass for the VL chain was detected.

Size-exclusion chromatography (SEC) separation by size and/or molecular weight was further used to determine protein expression. Size-exclusion chromatography (SEC) separation by size and/or molecular weight was further used to determine protein expression. Only two peaks were detected and the concentration of peak 2 was greater than 99% of the total combined size of peak 1 and peak 2. The humanized 6H5 antibody (purity >95%) with an endotoxin level<1 EU/mg was used to determine antitumor effects in vitro and in vivo.

An ELISA assay was used to compare antigen binding sensitivity and specificity of hu6H5 vs. m6H5. See FIG. 2. No significant difference between these two parameters was detected.

An apoptosis assay was used to compare the efficacy of hu6H5 and m6H5 in killing cancer cells. The respective antibodies (1 or 10 μg per ml) were used to treat MDA-MB-231 breast cancer cells for 4 hours and 24 hours. Apoptosis assays were used to determine the cytotoxicity of mouse and humanized anti-HERV-K antibodies toward cancer cells. Cancer cells including MDA-MB-231-pLVXK (231K) (a breast cancer cell line transduced with pLVX vector that expresses HERV-K env protein) or MDA-MB-231-pLVXC (231C) (the same breast cancer cell line transduced with pLVX empty vector) were treated with either m6H5 or hu6H5 (1 or 10 μg per ml) for 4 hours or 16 hours. Annexin V and 7AAD were used to determine the percentage of apoptotic cells.

Cells treated with no antibody or with mIgG or human IgG were used as control. The results showed that hu6H5 a similar effect as m6H5 at killing these breast cancer cells. To further evaluate efficacy in cell killing, MDA-MB-231 cells were treated with various antibodies (10 μg/ml) for sixteen hour. Live cells and putative dead cells were identified using the co-stained Live/Dead Viability Assay, and the results showed that hu6H5 had a similar effectiveness to m6H5 at killing breast cancer cells. Live/dead cell viability assays were used to assess induction of cell death after anti-HERV-K antibody treatment. MDA-MB-231 cells were seeded overnight in 24-well plates. Cells were treated with various antibodies (10 μg/ml) and incubated for sixteen hours at 37° C. in a cell culture incubator. Calcein Am (4 μl/10 ml media) and Eth-D1 (20 μl/10 ml media), 200 μl per well, were then added and cells were incubated for thirty minutes at room temperature. EthD-1 penetrates cells with membrane damage and binds to nucleic acids to produce red fluorescence in dead cells. Live cells (green color; Calcein Am) and putative dead cells (red color; EthD-1) were identified using the co-stained Live/Dead Viability Assay. Human IgG or mouse IgG were used control. No red fluorescent cells were observed after treatment with control human or mouse IgG. However, red fluorescent cells were observed in cells treated with humanized or mouse 6H5 anti-HERV-K antibodies.

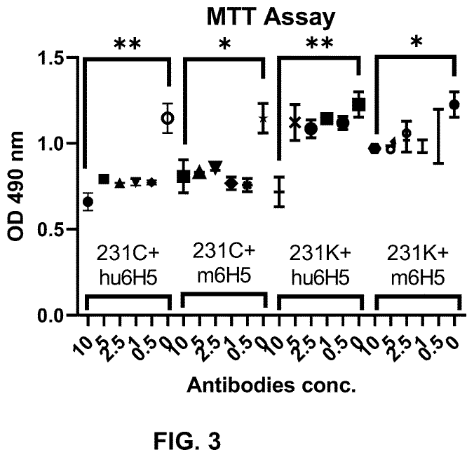

An MTT (3-(4,5-dimethylthiazol-2-yl)-2,5-diphenyltetrazolium bromide) assay was used to confirm that hu6H5 can inhibit cancer cell growth. See FIG. 3.

ADCC was used to determine the mechanism of BC cell killing, and our results support effector cell mediated secretion of cytotoxic molecules that lyse antibody-coated target cells. ADCC was used to determine the mechanism of antibody-induced cell killing. Greater ADCC lysis of cancer cells were observed in cells treated with hu6H5 than with m6H5, with increasing percentages of peripheral blood mononuclear cells.

Flow cytometry was used to determine if hu6H5 can downregulated the expression of p-ERK, Ras, and SIRT-1. 231C or 231K cells were treated with 10 μg per ml of hu6H5 for sixteen hours. The expression of HERV-K, SIRT-1, p-ERK and Ras in both cells under perm and non-perm. Down-regulated expression of HERV-K, p-ERK, Ras, and SIRT-1 was demonstrated in 231K treated with hu6H5 or 231C. Mice were inoculated with 231K or 231C cells (2 million by subcutaneous injection). The mice were then treated with hu6H5 antibody (n=3; 4 mg/kg, twice per week). Tumor growth was monitored and measured three times per week, and mouse survival was determined. Treatment of the mice with hu6H5 led to longer survival than treatment of another cohort of mice with the control antibody (n=4). Shorter survival was observed in mice inoculated with 231K cells than in mice inoculated with 231C cells, which indicates that overexpression of HERV-K in breast cancer cells shortens tumor-associated survival.

pLVXK is an HERV-K expression vector, and MDA-MB-231 pLVXK are MDA-MB-231 cells transduced with pLVXK. Likewise, pLVXC is control expression vector only, and MDA-MB-231 pLVXK are MDA-MB-231 cells transduced with pLVXC. NSG female mice (8-week-old), were inoculated with MDA-MB-231 pLVXC (231-C; subcutaneous, 2 million cells) vs, MDA_MB-231 pLVXK (231-K; subcutaneous, 2 million cells). On day 6, mice were treated with hu6H5 (4 mg/kg intraperitoneal, twice weekly for 3 weeks). Tumor growth was monitored and measured every other day. Higher survival was demonstrated in mice bearing 231-C and 231-K cells treated with antibodies. Tumor and lung tissues were collected from each mouse. Larger lymph nodes were detected in some mice bearing 231-K cells but not in mice bearing 231-C cells.

Hematoxylin and eosin (H&E) staining was further used to assess morphological features of tumor tissues and tissues from other organs (lungs and lymph nodes Tumor viability and tumor necrosis were quantitated by a pathologist by measuring the tumor areas by H&E staining. Humanized antibody treatment resulted in smaller tumor volumes, less tumor focality and number, less infiltrative borders, and decreased mitotic activities. A reduced percentage of tumor viability was observed in mice bearing 231-C cells or in 231-K cells treated with antibody. Reduced tumor variability was demonstrated in 231C or 231K cells treated with hu6H5, relative to their controls. Anti-Ki67 and anti-HERV-K mAb were used. Reduced tumor viability was demonstrated in mice treated with hu6H5 (20%) compared with control (60%). The antibody treatment groups were more uniform in appearance, with less pleomorphic nuclei and smaller nucleoli, and tumor-infiltrating lymphocytes were significantly increased in number.

Metastatic tumor cells were also found in lung tissues obtained from mice bearing 231-K cells, but not in mice bearing 231-C cells. Metastases to lung and lymph nodes were observed in mice inoculated with 231K cells. Metastases to lung or lymph nodes were observed only in mice inoculated with 231K cells. Reduced tumor viability and increased tumor necrosis was detected in lung of mice inoculated with 231K cells and treated with hu6H5. Visibly enlarged lymph nodes were seen in mice inoculated with 231K, but not in mice inoculated with 231C cells. Reduced tumor viability and increased tumor necrosis were detected in lymph nodes from mice inoculated with 231K cells and treated with hu6H5 (KAB) (B18; 40%) vs 231K cells with no added antibody (KCON) (B26>95%). These results show that HERV-K expression is a causal factor for tumor development, and especially for metastasis to distant organ sites. Our humanized anti-HERV-K antibody can reduce tumor viability, increase tumor necrosis, and decrease metastasis to the lungs and lymph nodes. Reduced percentages of tumor viability were observed in lungs of mice bearing 231-K treated with antibody compared to those not treated with antibody. Metastatic lymph nodes were detected only in mice inoculated with 231K cells. Greater than 95% tumor viability was detected in lymph nodes in mice bearing 231-K cells and reduced percentages of tumor viability were observed in lymph nodes in mice treated with antibody compared to mice not treated with antibody. Ascites were demonstrated in mice that carried 231K or 231C tumor cells without antibody treatment.

### Example 10

### Efficacy of a Bispecific T Cell Engager (BiTE) Targeting HERV-K

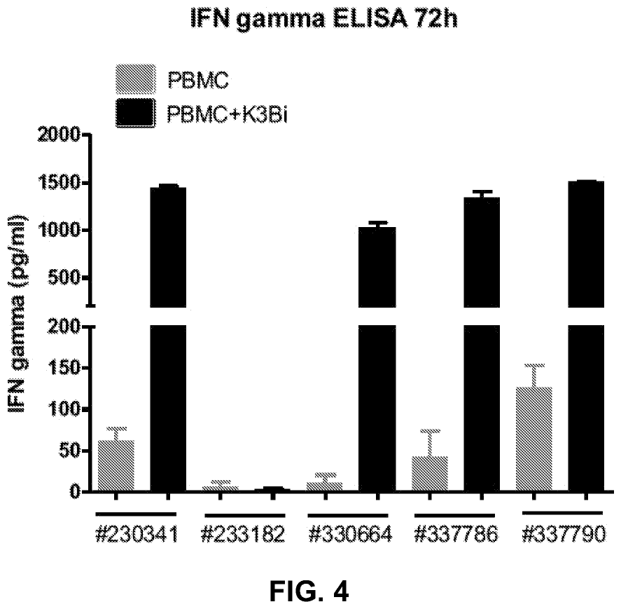

A BiTE directed against T cell CD3 or CD8 and the tumor-associated antigen HERV-K was produced, comprised of antibodies targeting either CD3 or CD8 and HERV-K. This BiTE was shown to elicit interferon-gamma (IFNγ) cytotoxic activity towards MDA-MB-231 breast cancer cells expressing major histocompatibility class (MHC) molecules loaded with HERV-K epitopes, with 20-30-fold increases in IFNγ expression after treatment with the BiTE. See FIG. 4.

A BiTE is a recombinant protein built as a single-chain antibody construct that redirects T cells to tumor cells, and that does not require expansion of endogenous T cells through antigen-presenting cells. BiTE molecules can be administered directly to patients and BiTE-mediated T cell activation does not rely on the presence of MHC class I molecules, as does CAR. Given the success of targeting HERV-K Env as a tumor-associated antigen (TAA), and that nearly all breast cancer cell lines express Kenv protein, the inventors hypothesize that a BiTE specific for Kenv and CD3 (K3Bi) effectively treats metastatic disease as did K-CAR. The inventors have designed and synthesized a K3Bi with dual specificity for Kenv and CD3. Thus, T cells target HERV-K+ tumor cells. The inventors have generated, purified, and validated the K3Bi and a CD8 BiTE (K8Bi). This was done using the mAb 6H5 that was also used in the CAR construct, and OKT3, an antibody against human CD3 used in other BiTEs, which was humanized and connected with a flexible linker plus two C-terminal epitope tags (MYC and FLAG) for purification and staining. A CD8 single chain antibody (scFv) obtained from OKT8 hybridoma cells was generated in the inventors' lab and used to produce K8Bi (VL-VH6H5 linker VH-VLCD8-MYC and FLAG). K3Bi and K8Bi were cloned into the pLJM1-EGFP Lenti or pGEX-6P-1vector for recombinant protein expression. The capacity of the K3Bi or K8Bi to bind to T cells and HERV-K⁺ breast cancer cell lines was determined by several immune assays. The inventors found that increased numbers of target cells bound to BiTE with increased BiTE concentrations.

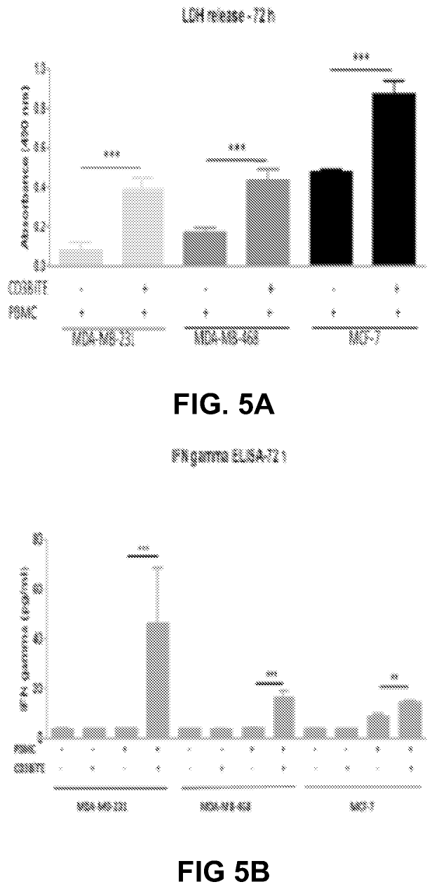

The inventors also examined the capacity of the K3Bi to induce T cell activation, proliferation, production of cytokines, and lysis of target tumor cells. Bulk PBMCs (50,000 per well) from healthy controls co-cultured with K3Bi (0, 1, 10, 100, and 1,000 ng/ml) and tumor cells (5,000 per well) to achieve effector cell: target cell ratios of 10:1 as described in Zhang et al., Cancer Immunol. Immunother., 63, 121-132 (2014). One result is shown in FIG. 5. PBMC+ MCF-7+ K3Bi exhibited increased cancer cell killing compared PBMC+ MCF-7 without K3Bi. See FIG. 5A. An LDH release assay was used for detection of cell viability and cytotoxicity, as the inventors did previously. See Zhou et al., Oncoimmunology, 4, e1047582 (2015). Enhanced IFNγ production, assayed by ELISA. See FIG. 5B was observed in MDA-MB-231, MDA-MB-468, and MCF-7 cells treated with K3Bi. Untreated cells, PBMC only, or BiTE only were used as controls and no IFNγ production was observed in these control groups.

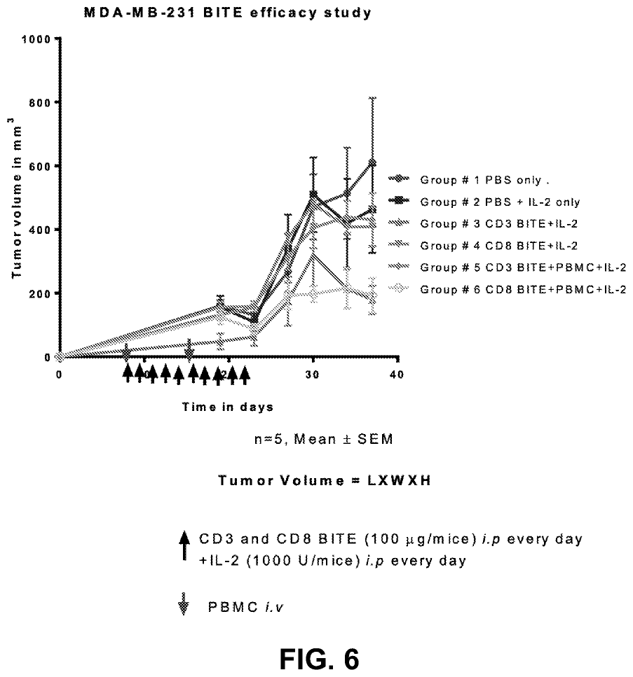

Treatment of immunodeficient NSG mice bearing HERV-K positive MDA-MB-231 breast cancer cells with PBMCs and CD3 HERV-K BiTE plus IL-2 or CD8 HERV-K BiTE plus PBMCs plus IL-2 resulted in greatly decreased tumor growth. See FIG. 6. Also, 231K cells were treated with hu6H5 vs human immunoglobulin G (huIgG) control antibody. A Live and Dead Cell Assay Kit (Calcein AM, 7-AAD) revealed that 231K cells exhibit increased killing after treatment with hu6H5 compared with control huIgG.

### Example 11

Staining Results of Normal Donor PBMC's Traduced with CAR-A and CAR-B Lentiviral Vectors

PBMCs from normal donors were transduced with two CAR-T lentiviral vector constructs, K-CAR-A (CAR-A) or K-CAR B (CAR-B). pWPT-GFP with psPAX2 and pMD2g. VH-VLhu6H5-CD8-CD28-4-1BB-CD3zeta. The protocol to generate HERV-Kenv CAR-T cells by an alternate to the Sleeping Beauty transduction process, namely lentiviral transduction, is:
1. Thaw PBMCs (2×107) and deplete monocytes by plastic adherence (one hour's incubation at 37° C., 5% C02).
2. Culture monocytes depleted PBMCs in RPMI 1640 supplemented with 10% FBS, 100 U/mL penicillin, 100 μg/mL streptomycin (complete medium). Stimulate T cells with anti-CD3/CD28 beads in a 3:1 bead:cell ratio with 40 IU/mL IL-2 for twenty-four hours.
3. Transduce activated T cells with CAR-A or CAR-B lentiviral particles (CD19 CAR as control).
4. Twenty-four hours after transducing, culture T cells in complete medium containing 300 IU/mL IL-2 and γ-irradiated (100 Gy) MDA-MB-231-Kenv (Kenv is the envelope protein of HERV-K) aAPC at a 2:1 aAPC/T cell ratio to stimulate CAR-T cell proliferation. Use γ-irradiated K562-CD19 as the control aAPC.
5. Remove Anti-CD3/CD28 beads on day 5. Replenish CAR-T cells with fresh media containing IL-2 every two-three days.
6. Use CAR-T cells when proliferation show a decrease from log-phase.

The CAR-A or CAR-B transduced cells were co-cultured with γ-irradiated (100 Gy) MDA MB 231 antigen presenting cells. Soluble IL-2 cytokine (50 U/ml) was added every other day. On day 14 the cells were harvested for staining. They were stained first for 20 minutes at 4° C. with a 1:1000 dilution of BV450 live and dead stain. After twenty minutes, the cells were washed and stained with K10-AF 488 protein (1 μg/ml), CD4 Amcyan, CD3 Pe cy7, and goat anti human IgG Fc AF 594 antibodies according to manufacturers' recommendations for thirty minutes at 4° C. and washed with phosphate-buffered saline. The cells were fixed with 4% PFA for fifteen-thirty minutes and washed before analyzing in a flow cytometer. The samples were positive for green fluorescent protein (GFP), as they were transfected with GFP+CAR-A/CAR-B.

The percentage of CD4+ cells was determined by gating those populations negative for BV450 and positive for respective colors. The percentage of CD4− (called CD8+ cells) were gated by selecting those populations negative for BV450 and negative for CD4 Amcyan color. The results show that the percentage of CD4+ PBMC's transduced with CAR-A/CAR-B that get stained with K10 labelled AF488 protein are higher than the percentage of naïve T cells that get stained with K10 labelled AF488 protein. This shows that T cells transduced with CAR-A or CAR-B are stained with the HERV-K10 protein.

T cells expressing a lentiviral CAR expression vector that bears a humanized or fully human HERV-K scFv will effectively lyse and kill tumor cells from several cancers. Humanized K-CARs expressed from lentiviral vectors are pan-cancer CAR-Ts.

### Example 12

### A HERV-K Specific Humanized Chimeric Antigen Receptor (K-CAR) Therapy

The inventors have produced a humanized single chain variable fragment (scFv) antibody (EXAMPLE 1), which bound antigens produced from recombinant HERV-K Env surface fusion protein (KSU) (EXAMPLE 3) and lysates from MDA-MB-231 breast cancer cells. A CAR produced from this humanized scFv is cloned into a lentiviral vector and is used combined with therapies that include but are not limited to K-CAR T cells plus checkpoint inhibitors, proinflammatory cytokines such as interleukin (IL)-12 and IL-18, oncolytic viruses, and kinase inhibitors (including but not limited to p-RSK, p-ERK).

### Example 13

Identification of Human Therapeutic Antibodies (hTAbs) from Very Rare B Cells that Exhibit Strong Target Specificity and High Sensitivity

Generation of fully human therapeutic antibodies from the human adaptive immune system: To directly use B cells from breast cancer patients as a source of high-affinity antibodies, the inventors performed an indirect ELISA or immunoblot with HERV-K Env recombinant fusion protein, which the inventors used to detect anti-HERV-K Env specific responses from several breast cancer patients. Patients with higher titers of anti-HERV-K antibodies were selected for single B cell assays. PBMCs from breast cancer patients were polyclonally activated: (1) using irradiated 3T3-CD40L fibroblasts for two weeks. This method can efficiently stimulate and expand CD40-B cells to large numbers in high purity (>90%) and induce secretion of their antibodies; and (2) ex vivo with recombinant human IL-21, IL-2, soluble CD40 ligand and anti-APO1 for four days. This second method can enable secretion from the highest percentage of B cells using minimal culture times. IL-21 promotes the differentiation to antibody-secreting cells. IL-2 stimulation in vitro can trigger human plasma cell differentiation, which requires T cell help to reach the induction threshold. sCD40L engages with CD40 expressed on the cell surface of B cells to mimic T cell-mediated activation. Since activation also induces cell death, anti-APO1 is used to rescue B cells from Fas-induced apoptosis. Few cytotoxic B cells were detected.

Development of a platform to determine the binding kinetics and cell-to-cell interactions of every cell in a microwell slab. Details of the microengraving process, which enables the screening and monitoring of B cell interactions over time to enable single-cell cloning of antibody-producing B cells. The arrays of nanowells in polydimethyl siloxane (PDMS) are fabricated, and cells from mammospheres from patient breast tumor tissues produced and cultured in the inventors' lab were used as targets for determining the efficacy of breast cancer cell killing. B cells and mammosphere cells (1:1 ratio) from the same donor were loaded onto a nanowell array (one cell per well) and the cells allowed to settle via gravity.—A dead tumor cell (red color) and B cell are shown in the same well. The anti-HERV-K antibody produced by this B cell was detected in the same position of the glass cover slide. The single B cell was then picked by a CellCelector for RT-PCR. Our results show that HERV-K specific memory B cells exhibited anti-HERV-K antibody expression and cytotoxicity toward their autologous mammosphere cells.

Therapeutic antibody discovery using an in vivo enrichment (IVE) adaptation: The platform will enable isolation of antibodies that not only bind target cancer cells but can also kill the cells. The platform will also enable the use of normal donors without memory B cells instead of breast cancer patient donors to generate hTAbs. Because B cells able to produce therapeutic antibodies for treatment are extremely rare even after ex vivo enrichment, the inventors developed the following platform to identify very rare hTAbs:

Groups (N=10/group) of wild type Balb/c mice (female, 6-week-old) were immunized on day 1 and boosted on week 3 and week 5. ELISPOT are used to determine IFNγ secretion by CD8+ T cells obtained from immunized mice (immunized with HERV-K transmembrane (TM) protein (mouse M1 to M4) or phosphate-buffered saline (M5 to M6). ELISA assays were used to detect the titers of anti-HERV-K IgG in immunized mouse sera. Higher titers of antibodies were detected in mice treated with HERV-K Env surface fusion protein regardless of CpG or CDN status. Anti-HERV-K antibody titers were detected by ELISA in human tumor mouse models inoculated with MDA-MB-231 (HTM1) or MDA-MB-468 (HTM2) and with humanized mice (HM1 and HM2) immunized with HERV-K surface fusion protein using anti-human IgG mAb.

EXAMPLE 13.1. Adapt an in vivo enrichment technique (IVE: ≈20-fold enhancement) in SCID/beige mice, allowing for rapid expansion and B cell activation, to produce large numbers of antigen-specific plasmablasts. See FIG. 4. This platform will produce fully human antibodies from B cells in as short a time as eight days. As a proof of principle, the inventors developed an in vivo enrichment technique to produce fully human anti-Zika antibodies in hybridoma cells generated from splenocytes on day 8 fusion with MFP-2 partner cells.

Recently, humanized mice (HM) and human tumor mice (HTM) were successfully generated by intravenous injection of CD34+ cells (1-2×10⁵/mouse) for humanized mice generation and immunization with HERV-K SU or PD-L1 recombined fusion proteins.

The inventors also co-implanted CD34+ hematopoietic stem cells with 5×10⁴-3×10⁶ breast cancer cells triple negative breast cancer patient derived xenografts (TNBC PDX cells, or MDA-MB-231 or MDA-MB-468 TNBC cells) in the mammary fat pad for human tumor mouse generation. The percentages of CD33, CD3, and CD19⁺ cells were quantified in huCD45⁺ cells obtained at four weeks post-inoculation of TNBC PDX cells, and in the MDA-MB-231 human tumor mouse model at seven weeks post-inoculation, with co-implantation of CD34⁺ hematopoietic stem cells. The percentage of hCD19 or hCD45 cells is higher in mice after a longer period of post-inoculation with CD34 cells. Exposure to antigen was associated with HERV-K expression in the tumor, and a higher antibody titer was detected (HTM2: 40 days vs. HTM1: 30 days). This result indicates that human tumor mice can produce anti-HERV-K antibodies in mice inoculated with breast cancer cells.

This result prompted the use of humanized mice or human tumor mice to generate fully hTAbs, and especially to use normal donors who were never exposed to antigen. NSG mice, which lack T-cell, B-cell, and NK-cell activity, are ideal candidates to establish humanized mice. Immunofluorescence staining was used to detect the expression of HERV-K using anti-HERV-K mAb 6H5 in an MDA-MB-231 tumor obtained from a human tumor mouse. F-actin was the control. huCD3⁺ cells were also detected in tumor tissues. Anti-HERV-K antibody titers were detected by ELISA in human tumor mice models inoculated with MDA-MB-231 (HTM1) or MDA-MB-468 (HTM2) and with HM1 and HM2 immunized with HERV-K SU Env protein using anti-human IgG mAb.

Mice with a higher engraftment rate of human CD45+ cells than was seen in earlier results, with no significant toxicity, were later developed.

In Vivo Enrichment Protocol 1. For donors with cancer with a higher titer of antibodies, the inventors use the protocol using humanized mice instead the standard SCID/beige mice. PBMCs (50×10⁶) from breast cancer patients are polyclonally activated by IL-21, IL-2, soluble CD40 ligand and anti-APO1, and premixed with antigens (HERV-K or PD-L1; 100 μg). B cells isolated from the above PBMCs using an EasySep™ Human B Cell Enrichment Kit (Stemcell Technologies) by negative selection are co-injected with CD34 cells in the mice treated with busulfan (Fisher: 30 mg/kg intraperitoneally) on day 0. Mice are treated with cytokine cocktails (days 1, 4, and 7) and boosted by antigens on day 2. This protocol can be completed relatively quickly (8 days).

In Vivo Enrichment Protocol 2. For normal donors without cancer and who have no memory B cells, the inventors use In Vivo Enrichment Protocol 1 with modifications: Mice are treated with cytokine cocktails (days 1, 7, and 14) and boosted by antigens on day 14 and day 21. Sera are collected from mice and binding affinity is tested by ELISA. After increased antibody titers are detected, spleens are harvested, analyzed, and used to make hybridomas. Higher antibody titers were detected in mice using In Vivo Enrichment Protocol 2 on week 2.

EXAMPLE 13.2. After, half of the spleen is harvested and used for flow cytometric analysis, microengraving and other analyses. Flow cytometric analysis of B cell surface and intracellular markers and CFSE labeling (Invitrogen CellTrace CFSE kit) is performed using: Anti-CD19 PECy5, anti-CD27 allophycocyanin, anti-CD38 PECy7, anti-IgG FITC, or anti-IgM PE isotype controls of mouse IgG1k conjugated to FITC, PE, PECy5, PECy7, Alexa 700, or allophycocyanin (all from BD Bioscience). Negative magnetic immunoaffinity bead separation (Miltenyi Biotec) is used to isolate total CD19+ B cells from spleen and stimulate with CpG2006 (10 ng/ml; Oligos, Inc.) in the presence of recombinant human B cell activating factor (BAFF; 75 ng/ml; GenScript), IL-2 (20 IU/ml), IL-10 (50 ng/ml), and IL-15 (10 ng/ml) (all from BD Biosciences) for seventy-two hours. Tumor-killing B cells directly from Protocol 1 or Protocol 2 are determined using our multi-well microengraving platform (up to 400,000 wells), with their autologous tumor cells or HERV-K+ TNBC cells as target cells. Cells that not only produce antibodies but can also bind antigen and kill cancer cells are determined.

EXAMPLE 13.3. The inventors then develop human hybridoma cells to ensure long-term antibody availability. To develop a fully human hybridoma, MFP-2 cells are used as a partner to generate hybridomas with the remaining half of the spleen using ClonaCell™-HY (Stemcell Technologies Inc.) following their protocol. Polyethylene glycol (PEG) is used for fusing human lymphocytes with MFP-2 cells and a methylcellulose-based semi-solid media in this kit is used for cloning and selection of hybridoma cells. The clones that grow out after selection are pipetted into 96-well plates and screened for reactivity to HERV-K Env protein by ELISA. The positive clones' isotypes are determined using a Human IgG Antibody Isotyping Kit from Thermo Fisher Scientific. The clones are then adapted to serum-free media conditions and expanded. Hybridoma supernatant is harvested, and antibody is purified using Hi-Trap protein A or protein G columns, depending on the isotype of the human antibody. Protein A columns have high affinity to antibodies of the isotype-IgG1, IgG2, and IgG4, and variable binding to antibodies of the isotype IgM, whereas Protein G columns exhibit high binding to antibodies of the isotype-IgG1, IgG2, IgG3, and IgG4, but do not bind IgM antibodies.

EXAMPLE 13.4. The inventors evaluate the antitumor efficacy of candidate B cells obtained from the above protocols in vitro, including effects on cell growth, proliferation, and apoptosis, as the inventors do routinely in our lab. In vivo studies to evaluate the efficacy of the hTAbs in immunodeficient mouse models are also done to evaluate efficacy, using breast cancer cell lines and primary tumor cells, and compared with matched uninvolved control breast cells.

### Example 14

### Combination Therapy

The inventors' breast cancer data from strongly support the use of combination therapy approaches involving HERV-K. Humanized and fully human antibodies targeting HERV-K should therefore enhance checkpoint blockade antibody treatment efficacy. Effective combined cancer therapies include but are not limited to combinations of (a) HERV-K hTAb (1.5 mg/kg), (b) K-CAR, (c) K-BiTE, (d) HERV-K shRNAs or CRISPR/Cas9 genome editing technology to knock down HERV-K gene expression, (e) or preventive or therapeutic HERV-K vaccines, including full-length and truncated HERV-K Env proteins and HERV-K Env peptides, and (a) anti-ICP antibody, (b) cancer chemotherapy, (c) 5-azacytidine, 5-aza-2′-deoxycytidine, or other epigenetic modulating agents, such as DNA methyltransferase inhibitors (DNMTi) and histone deacetylase inhibitors (HDACi), (d) EMT inhibitors, (e) inhibitors of cell migration or invasion, (f) induction of S or G2 phase cell cycle arrest, (g) inhibitors of PI3K/AKT/mTOR or MAPK/ERK signaling pathways, or (h) signal transduction to HIF1α.

The inventors assessed the baseline immune status in relation to HERV-K status in breast cancer patients using combined HERV-K and immune checkpoint assays. Expression of soluble immune checkpoint proteins was determined by Luminex assay in breast cancer patients including DCIS and aggressive breast cancer vs. normal donors. The inventors made comparison of expression of six ICPs in DCIS, aggressive breast cancer (aBC), and normal female donors. A striking finding was a significantly enhanced expression of the six circulating ICPs in the plasma of breast cancer patients A further finding was a marked drop in immune checkpoint protein levels in patients at six months or eighteen months post-surgery vs. pre-surgery (Timepoint 1). A positive association between soluble ICP molecule levels and HERV-K antibody titers induced by HERV-K expression in the tumor was observed. This result suggests that HERV-K antibody titers should influence ICP levels in breast cancer. The expression of HERV-K can thus control immune responses of breast cancer patients.

### Example 15

Sequence data anti-CD3 and CD8 BiTE

TABLE 3
Anti-CD8 Heavy chain alignment

| | | |
|---|---|---|
| H3 | TNTCAGGTGCAGCTGAAGCAGTCTGGGGCAGAGCTTGTGAAGCCAGGGGCCTCAGTCAAG | 60 |
| H6 | TTTGAGGTCCAGCTGCAGCAGTCTGGGGCAGAGCTTGTGAAGCCAGGGGCCTCAGTCAAG | 60 |
| H7 | --TCAGGTCCAACTGCAGCAGCCTGGGGCAGAGCTTGTGAAGCCAGGGGCCTCAGTCAAG | 58 |
| H1 | TTTGAGGTCCAGCTGCAGCAGTCTGGGGCAGAGCTTGTGAAGCCAGGGGCCTCAGTCAAG | 60 |
| | * **** **.***.***** ************************************** | |
| H3 | TTGTCCTGCACAGCTTCTGGCTTCAACATTAAAGACACCTATATACACTTCGTGAGGCAG | 120 |
| H6 | TTGTCCTGCACAGCTTCTGGCTTCAACATTAAAGACACCTATATACACTTCGTGAGGCAG | 120 |
| H7 | TTGTCCTGCACAGCTTCTGGCTTCAACATTAAAGACACCTATATACACTTCGTGAGGCAG | 118 |
| H1 | TTGTCCTGCACAGCTTCTGGCTTCAACATTAAAGACACCTATATACACTTCGTGAGGCAG | 120 |
| | ********************************************************** | |
| H3 | AGGCCTGAACAGGGCCTGGAGTGGATTGGAAGGATTGATCCTGCGAATGATAATACTTTA | 180 |
| H6 | AGGCCTGAACAGGGCCTGGAGTGGATTGGAAGGATTGATCCTGCGAATGATAATACTTTA | 180 |
| H7 | AGGCCTGAACAGGGCCTGGAGTGGATTGGAAGGATTGATCCTGCGAATGATAATACTTTA | 178 |
| H1 | AGGCCTGAACAGGGCCTGGAGTGGATTGGAAGGATTGATCCTGCGAATGATAATACTTTA | 180 |
| | ********************************************************** | |
| H3 | TATGCCTCAAAGTTCCAGGGCAAGGCCACTATAACAGCAGACACATCATCCAACACAGCC | 240 |
| H6 | TATGCCTCAAAGTTCCAGGGCAAGGCCACTATAACAGCAGACACATCATCCAACACAGCC | 240 |
| H7 | TATGCCTCAAAGTTCCAGGGCAAGGCCACTATAACAGCAGACACATCATCCAACACAGCC | 238 |
| H1 | TATGCCTCAAAGTTCCAGGGCAAGGCCACTATAACAGCAGACACATCATCCAACACAGCC | 240 |
| | ********************************************************** | |
| H3 | TACATGCACCTCTGCAGCCTGACATCTGGGGACACTGCCGTCTATTACTGTGGTAGAGGT | 300 |
| H6 | TACATGCACCTCTGCAGCCTGACATCTGGGGACACTGCCGTCTATTACTGTGGTAGAGGT | 300 |
| H7 | TACATGCACCTCTGCAGCCTGACATCTGGGGACACTGCCGTCTATTACTGTGGTAGAGGT | 298 |
| H1 | TACATGCACCTCTGCAGCCTGACATCTGGGGACACTGCCGTCTATTACTGTGGTAGAGGT | 300 |
| | *********************************************************** | |
| H3 | TATGGTTACTACGTATTTGAC-CACTGGGGCCAAGGCACCAC---TCTCACANTNNCNNN | 356 |
| H6 | TATGGTTACTACGTATTTGAC-CACTGGGGCCAAGGCACCAC---TCTCACANTNNCNNN | 356 |
| H7 | TATGGTTACTACGTATTTGAC-CACTGGGGCCAAGGCACCAC---TCTCACATNTNNNTN | 354 |
| H1 | TATGGTTACTACGTATTTGACTCACTTGGGCCAAGGCNNTNNNNNTNNCANNNTNNNNNN | 360 |
| | ********************* **** *********** .**...* *.* | |
| H3 | N- | 357 (SEQ ID NO: 23) |
| H6 | A- | 357 (SEQ ID NO: 25) |
| H7 | -- | 354 (SEQ ID NO: 27) |
| H1 | NN | 362 (SEQ ID NO: 21) |

TABLE 4
Anti-CD8 Light chain alignment

| | | |
|---|---|---|
| K4 | TTTTGACATTGTGCTGACCCAATCTCCTGCTTCCTTAGCTGTATCTCTGGGGCAGAGGGC | 60 |
| K6 | TNTTGATATTGTGCTAACTCAGTCTCCTGCTTCCTTAGCTGTATCTCTGGGGCAGAGGGC | 60 |
| K8 | ---TGACATCCAGCTGACTCAGTCTCCTGCTTCCTTAGCTGTATCTCTGGGGCAGAGGGC | 57 |
| | *** **:***.** **.****************************************** | |
| K4 | CACCATCTCATACAGGGCCAGCAAAAGTGTCAGTACATCTGGCTATAGTTATATGCACTG | 120 |
| K6 | CACCATCTCATACAGGGCCAGCAAAAGTGTCAGTACATCTGGCTATAGTTATATGCACTG | 120 |
| K8 | CACCATCTCATACAGGGCCAGCAAAAGTGTCAGTACATCTGGCTATAGTTATATGCACTG | 117 |
| | ********************************************************** | |
| K4 | GAACCAACAGAAACCAGGACAGCCACCCAGACTCCTCATCTATCTTGTATCCAACCTAGA | 180 |
| K6 | GAACCAACAGAAACCAGGACAGCCACCCAGACTCCTCATCTATCTTGTATCCAACCTAGA | 180 |
| K8 | GAACCAACAGAAACCAGGACAGCCACCCAGACTCCTCATCTATCTTGTATCCAACCTAGA | 177 |
| | ************************************************************ | |
| K4 | ATCTGGGGTCCCTGCCAGGTTCAGTGGCAGTGGGTCTGGGACAGACTTCACCCTCAACAT | 240 |
| K6 | ATCTGGGGTCCCTGCCAGGTTCAGTGGCAGTGGGTCTGGGACAGACTTCACCCTCAACAT | 240 |
| K8 | ATCTGGGGTCCCTGCCAGGTTCAGTGGCAGTGGGTCTGGGACAGACTTCACCCTCAACAT | 237 |
| | *********************************************************** | |
| K4 | CCATCCTGTGGAGGAGGAGGATGCTGCA-ACCTATTA-CTGTCAGCACATTA-GGGAGCT | 297 |
| K6 | CCATCCTGTGGAGGAGGAGGATGCTGCA-ACCTATTA-CTGTCAGCACATTA-GGGAGCT | 297 |
| K8 | CCATCCTGTGGAGGAGGAGGATGCTGCTGACCTATTAGCTGTCAGCACATTATGGGAGCT | 297 |
| | ***************************: ******** ************* ******* | |
| K4 | TACACGTTCGGAGGGGGACCAAGCNNNNNNN--- | 328 (SEQ ID NO: 29) |
| K6 | TACACGNNGANAANNNNNNNNNNCNNNNTNNNNN | 331 (SEQ ID NO: 31) |
| K8 | TACACGTTCGGNNNNNTNNCTNNTNNNNNNNNNC | 331 (SEQ ID NO: 33) |
| | ******: | |

### Order of the IgG Domains:

VL-VH6H5-VH-VLhuCD3 or CD8+c-myc tag+FLAG or VL-VHhu6H5-VH-VLhuCD3 or huCD8+c-myc tag+FLAG.
CD8 BiTE: See SEQ ID NOs: 35-36.
CD3 BiTE: See SEQ ID NOs: 37-38.

Mice were immunized with five MAPs and sera were collected and tested by ELISA using various HERV fusion proteins. Only HERV-K surface fusion protein was positive. Hybridoma cells were generated from the mice immunized with five MAPs and a scFv was selected. For scFv against MAPs of HERV-K (sequence for anti-HERV-K mAb), see SEQ ID NOs: 45-46.

### Example 16

Humanized Antibodies Targeting HERV-K can be Used for Antibody-Drug Conjugates to Deliver the Drugs into Cancer Cells and Tumors

Delivery of the recombinant gelonin toxin (r-Gel) was observed in HERV-K positive cancer cells using the anti-HERV-K 6H5-rGel antibody-drug conjugate.

Recombinant gelonin (r-Gel) toxin was conjugated with 6H5. Surface and cytoplasmic expression of HERV-K in DOV13 ovarian cancer cells was detected using anti-HERV-K 6H5 mAb. r-Gel expression was detected in DOV13 cells using anti-rGel antibodies after four hours treatment.

HERV-K env protein or r-Gel signals were detected in OVCAR3, SKBr3, MCF-7, and MDA-MB-231 cells after one hour internalization using anti-r-Gel antibody. The inventors observed colocalization of HERV-K env protein and rGel toxin in the target cell cytoplasm. Antitumor effects were compared in mice inoculated with MDA-MB-231 cells treated with 6H5 (p=0.0052), and 6H5-r-Gel (p<0.0001) compared with mice treated with control IgG

Gold nanoparticles (GNPs) were detected after two hours incubation with naked gold nanoparticle or 6H5-GNP by transmission electron microscopy (TEM) in MDA-MB-231 cells. Gold nanoparticles were detected in MDA-MB-231 or SKBr3 of tumors isolated from mice twenty-four hours post-intravenous-injection with the 6H5-GNP or 6H5scFV-GNP using a silver enhancement assay. Gold nanoparticles generate heat that kills targeted tumor cells when placed in a radiofrequency field.

### Example 17

### In Vivo Imaging of Anti-HERV-K Antibodies in Tumor Nodules of Mice

A higher density of 6H5 was detected in tumor nodules from mice twenty-four hours post-intravenous-injection with the anti-HERV-K-Alexa647 conjugate 6H5-Alexa647 by in vivo imaging using a Nuance system.

### LIST OF EMBODIMENTS

Specific compositions and methods of HERV-K antibody therapeutics. The scope of the invention should be defined solely by the claims. A person having ordinary skill in the biomedical art will interpret all claim terms in the broadest possible manner consistent with the context and the spirit of the disclosure. The detailed description in this specification is illustrative and not restrictive or exhaustive. This invention is not limited to the particular methodology, protocols, and reagents described in this specification and can vary in practice. When the specification or claims recite ordered steps or functions, alternative embodiments might perform their functions in a different order or substantially concurrently. Other equivalents and modifications besides those already described are possible without departing from the inventive concepts described in this specification, as persons having ordinary skill in the biomedical art recognize.

All patents and publications cited throughout this specification are incorporated by reference to disclose and describe the materials and methods used with the technologies described in this specification. The patents and publications are provided solely for their disclosure before the filing date of this specification. All statements about the patents and publications' disclosures and publication dates are from the inventors' information and belief. The inventors make no admission about the correctness of the contents or dates of these documents. Should there be a discrepancy between a date provided in this specification and the actual publication date, then the actual publication date shall control. The inventors may antedate such disclosure because of prior invention or another reason. Should there be a discrepancy between the scientific or technical teaching of a previous patent or publication and this specification, then the teaching of this specification and these claims shall control.

When the specification provides a range of values, each intervening value between the upper and lower limit of that range is within the range of values unless the context dictates otherwise.

Among the embodiments provided in this specification are:

1. An isolated antibody that binds to human endogenous retrovirus-K (HERV-K), comprising a heavy chain variable region (HCVR) and a light chain variable region (LCVR). Humanized anti-HERV-K antibody is able reduce tumor growth, especially reduce metastasis to lung, lymph nodes and other organs.

2. The antibody according to embodiment 1, comprising a humanized or human framework region.

3. The antibody according to embodiment 1, wherein the antibody is an HERV-K antagonist.

4. An isolated nucleic acid comprising a nucleotide sequence encoding the HCVR, the LCVR, or a combination thereof of embodiment 1.

5. An expression vector comprising the nucleic acid of embodiment 4.

6. A host cell transformed with an expression vector of embodiment 5.

7. A method of producing an antibody comprising a HCVR, a LCVR, or a combination thereof, the method comprising: growing the host cell of embodiment 1, under conditions such that the host cell expresses the antibody comprising the HCVR, the LCVR, or a combination thereof; and isolating the antibody comprising the HCVR, the LCVR, or combination thereof.

8. A method of treating cancer in a mammal, comprising administering an effective amount of the antibody according to embodiment 1 to a mammal in need thereof.

9. A method for treating cancer comprising administering, to an individual in need thereof, an effective amount of an antibody-drug conjugate comprising an antibody comprising a heavy chain variable region (VH) and a light chain variable region (VL), wherein: the VH region comprises a CDR1, a CDR2, and a CDR3, and the VL region comprises a CDR1, a CDR2, and a CDR3, wherein the antibody is conjugated to a cytotoxic drug, an auristatin or a functional peptide analog or derivate thereof via a linker.

10. The method of embodiment 9, wherein the antibody-drug conjugate is administered combined with one or more additional therapeutic agents.

11. The method of embodiment 9, wherein the one or more additional therapeutic agents includes a chemotherapeutic agent.

12. The method of embodiment 9, wherein the cancer is selected from the group consisting of melanoma, chronic lymphocytic leukemia, breast cancer, pancreatic cancer, head and neck cancer, ovarian cancer, cervical cancer, colorectal cancer, testicular cancer, stomach cancer, kidney cancer, endometrial cancer, uterine cancer, bladder cancer, prostate cancer, esophageal cancer, liver cancer, and non-small cell lung cancer.

13. The humanized antibody developed for CAR T, CAR NK, and BiTE studies.

14. The method of embodiment 9, wherein the antibody is a full-length antibody.

15. The method of embodiment 9, wherein the antibody is a human monoclonal IgG1 or IgG4 antibody.

16. The method of embodiment 9, wherein the auristatin is monomethyl auristatin E (MMAE) or monomethyl auristatin F (MMAF).

17. The method of embodiment 9, wherein the cytotoxic drug is emtansine (DM1); ozagamicin (calicheamicin); deruxtecan (DXd); govitecan (SN-38); mafodotin (MMAF); duocarmazine (duocarmycin); BAT8001 (maytansinoid) soravtansine (DM4); or tesirine (PBD).

18. The method of embodiment 9, wherein the linker is attached to sulphydryl residues of the antibody obtained by partial reduction of the antibody.

19. The method of embodiment 9, wherein the linker-auristatin is vcMMAF or vcMMAE.

20. The early detection, metastasis, or HERV-K plus immune checkpoint biomarkers, substantially as described herein.

21. Cancer cells overexpressing HERV-K as targets for the anti-HERV-K humanized antibodies and antibody-drug conjugates of the invention.

22. The hu6H5 clones (FWJ1 and FWJ2) generated from bacteria (HUM1 and HUM2) or mammalian cells.

23. A BiTE directed against T cell CD3 or CD8 and a humanized scFv against the tumor-associated antigen HERV-K, comprising antibodies targeting either CD3 or CD8 and HERV-K.

24. T cells expressing a lentiviral CAR expression vector that bears a humanized or fully human HERV-K scFv.

25. A humanized single chain variable fragment (scFv) antibody able to bind antigens produced from recombinant HERV-K Env surface fusion protein (KSU) and lysates from cancer cells expressed HERV-K Env proteins.

26. A CAR produced from the humanized scFv of embodiment 28, which is optionally cloned into a lentiviral vector.

27. A CAR produced from the humanized scFv of embodiment 28, which is cloned into a lentiviral vector, for use in combination therapies.

28. An improved in vivo enrichment method for rapid expansion and B cell activation for donors who have no memory B cells, comprising the steps of: treating mice with cytokine cocktails on days 1, 7, and 14, and boosting the mice by antigens on days 14 and 21.

29. Cells that not only produce antibodies, but the antibodies are also able to bind antigen and kill cancer cells, and cells that express the antigen can be killed by the antibodies.

30. A significantly enhanced expression of six circulating immune checkpoint proteins in the plasma of breast cancer patients.

31. A method of blockading of the immunosuppressive domain (ISD) with immune checkpoint inhibitors of HERV-K.

32. The method of embodiment 31, wherein the immune checkpoint inhibitors of HERV-K are selected from the group consisting of monoclonal antibodies and drugs targeting the ISD of HERV-K.

33. Humanized and fully human antibodies targeting HERV-K, for use in enhancing checkpoint blockade antibody treatment efficacy.

34. A method to produce new antibodies from mice immunized with 5 multiple antigen peptides (MAPS) that are generated from HERV-K SU protein produced by cancer patients.

35. A method to produce to produce HERV-K CAR A: VH-VLhu6H5-CD8-CD28-4-1 BB-CD3zeta.

### REFERENCES

A person having ordinary skill in the molecular biological art of can use these patents, patent applications, and scientific references as guidance to predictable results when making and using the invention:

### Patent References

U.S. Pat. No. 9,243,055 (Wang-Johanning). This patent discloses and claims cancer diagnostics and therapy. Methods and compositions for detecting, preventing, and treating HERV-K+ cancers are provided. One method is for preventing or inhibiting cancer cell proliferation by administering to a subject a cancer cell proliferation blocking or reducing amount of a HERV-K env protein binding antibody.
U.S. Pat. No. 10,723,787
International Pat. Publ. WO 2010/138803 (Board of Regents, the University of Texas System) discloses an isolated antibody that hinds to human endogenous retrovirus-K (HERV-K), comprising a heavy chain variable region (HCVR) and a light chain variable region (LCVR) (HERV-K protein recognized by an antibody, with a light chain variable region and a heavy chain variable region.
International Pat. Publ. WO 2014/186469 (Board of Regents, the University of Texas System). This patent publication concerns methods and compositions for immunotherapy using a modified T cell comprising a chimeric antigen receptor (CAR). CAR-expressing T-cells are produced using electroporation with a transposon-based integration system to produce a population of CAR-expressing cells that require minimal ex vivo expansion or that can be directly administered to patients for cancer treatment.
International Pat. Publ. WO 2019/104037 A1 (The Brigham and Women's Hospital, Inc.)

### Scientific References

Burstein & Winer, Refining therapy for human epidermal growth factor receptor 2-positive breast cancer: T stands for trastuzumab, tumor size, and treatment strategy. J. Clin Oncol., 27, 5671-5673 (2009).
Buscher et al., Expression of human endogenous retrovirus K in melanomas and melanoma cell lines. Cancer Res., 65, 4172-4180 (2005).
Cao, Human endogenous retroviruses in clear cell renal cell carcinoma: biological functions and clinical values.” Duvtlμress Web (Aug. 7, 2020).
Chae et al., Epithelial-mesenchymal transition (EMT) signature is inversely associated with T-cell infiltration in non-small cell lung cancer (NSCLC). Sci Rep., 8, 2918 (2018).
Chiappinelli et al., Inhibiting DNA Methylation Causes an Interferon Response in Cancer via dsRNA Including Endogenous Retroviruses. Cell., 162, 974-986 (2015).
Choi et al., Human B cell development and antibody production in humanized NOD/SCID/IL-2Rgamma(null) (NSG) mice conditioned by busulfan. J. Clin Immunol., 31, 253-264 (2011).
Denne et al., Physical and functional interactions of human endogenous retrovirus proteins Np9 and rec with the promyelocytic leukemia zinc finger protein. J. Virol., 81, 5607-5616 (2007).
Downey et al., Human endogenous retrovirus K and cancer: Innocent bystander or tumorigenic accomplice? Int. J. Cancer, 137, 1249-1257 (2015).
Ejthadi et al., A novel multiplex RT-PCR system detects human endogenous retrovirus-K in breast cancer. Arch Virol., 150, 177-184 (2005).
Elgueta et al., Molecular mechanism and function of CD40/CD40L engagement in the immune system. Immunol Rev., 229, 152-172 (2009).
Etkind, Lumb, Du, & Racevskis, Type 1 HERV-K genome is spliced into subgenomic transcripts in the human breast tumor cell line T47D. Virology., 234, 304-308 (1997).
Ettinger et al., IL-21 induces differentiation of human naive and memory B cells into antibody-secreting plasma cells. J. Immunol., 175, 7867-7879 (2005).
Feldhaus et al., Flow-cytometric isolation of human antibodies from a nonimmune Saccharomyces cerevisiae surface display library. Nature Biotechnol., 21, 163-170 (2003).
Fischer et al., Human endogenous retrovirus np9 gene is over expressed in chronic lymphocytic leukemia patients. Leuk. Res. Rep., 3, 70-72 (2014).
Gonzalez-Cao et al., Human endogenous retroviruses and cancer. Cancer Biol. Med., 13, 483-488 (2016).
Good, Bryant, & Tangye, Kinetics of human B cell behavior and amplification of proliferative responses following stimulation with IL-21. J. Immunol., 177, 5236-5247 (2006).
Grandi, HERV envelope proteins: Physiological role and pathogenic potential in cancer and autoimmunity. Frontiers in Microbiology. Web (Mar. 14, 2018).
Helsen et al. The chimeric TAC receptor co-opts the T cell receptor yielding robust anti-tumor activity without toxicity. Nature Commun., 9, 3049 (2018).
Herve et al., Autoantibodies to human endogenous retrovirus-Kare frequently detected in health and disease and react with multiple epitopes (Apr. 28, 2002) discloses antibodies specific to HERV-K (autoantibodies specific to HERV-K).
Hipp et al., IL-2 imprints human naive B cell fate towards plasma cell through ERK/ELK1-mediated BACH2 repression. Nature Commun., 8, 1443 (2017).
Hughes & Coffin, Evidence for genomic rearrangements mediated by human endogenous retroviruses during primate evolution. Nature Genetics, 29, 487-489 (2001). HERVs originated from thousands of ancient integration events which incorporated retrovirus DNA into germline cells.
Johanning et al., Expression of human endogenous retrovirus-K is strongly associated with the basal-like breast cancer phenotype. Science Reports, 7, 41960 (2017). The inventors observed the expression of HERVs, especially HERV-K sequences, in breast, lung, prostate, ovarian, colon, pancreatic, and other solid tumors. They also found that the expression of HERV-K env transcripts in breast cancer was specifically associated with basal breast cancer, an aggressive subtype.
Kleiman et al. HERV-K(HML-2) GAG/ENV antibodies as indicator for therapy effect in patients with germ cell tumors. Int. J. Cancer, 110, 459-461 (2004).
Koncz & Hueber, The Fas/CD95 receptor regulates the death of autoreactive B Cells and the selection of antigen-specific B cells. Front Immunol., 3, 207 (2012).
Kontermann & Brinkmann, Bispecific antibodies. Drug Discov. Today (2015).
Kontsekova et al., First-in-man tau vaccine targeting structural determinants essential for pathological tau-tau interaction reduces tau oligomerisation and neurofibrillary degeneration in an Alzheimer's disease model. Alzheimers Res. Ther., 6, 44 (2014).
Kraus et al. Vaccination directed against the human endogenous retrovirus-K envelope protein inhibits tumor growth in a murine model system. PLoS One, 8, e72756 (2013).
Krishnamurthy et al., Genetic Engineering of T Cells to Target HERV-K, an Ancient Retrovirus on Melanoma. Clin Cancer Res., 21, 3241-3251 (2015). The inventors observed the expression of HERVs, especially HERV-K sequences, in breast, lung, prostate, ovarian, colon, pancreatic, and other solid tumors.
Lander et al. Initial sequencing and analysis of the human genome. Nature, 409, 860-921 (2001). HERVs are well-known as genomic repeat sequences, with many copies in the genome, such that approximately 8% of the human genome is of retroviral origin.
Lanzavecchia, Corti, & Sallusto, Human monoclonal antibodies by immortalization of memory B cells. Current Opinion Biotechnol., 18, 523-528 (2007).
Larsson, Kato, & Cohen, Human endogenous proviruses. Current Topics Microbiol. Immunol., 148, 115-132 (1989). The most biologically active HERVs are members of the HERV-K family. HERV-K has a complete sequence capable of expressing all the elements needed for a replication-competent retrovirus.
Lemaitre et al, human endogenous retrovirus-derived gene that can contribute to oncogenesis by activating the ERK pathway and inducing migration and invasion. PLoS Pathog., 13, e1006451 (2017).
Li et al., Down-regulation of human endogenous retrovirus type K (HERV-K) viral env RNA in pancreatic cancer cells decreases cell proliferation and tumor growth. Clinical Cancer Research, 23, 5892-5911 (2017). The inventors observed the expression of HERVs, especially HERV-K sequences, in breast, lung, prostate, ovarian, colon, pancreatic, and other solid tumors.
Liu et al., Structure-based programming of lymph-node targeting in molecular vaccines. Nature., 507, 519-522 (2014).
Maldini, Dual CD4-based CART cells with distinct costimulatory domains mitigate HIV pathogenesis in vivo 1. Nature Medicine Web (Aug. 31, 2020).
Mazor et al., Isolation of engineered, full-length antibodies from libraries expressed in Escherichia coli. Nature Biotechnol., 25, 563-565 (2007).
Meijer et al., Isolation of human antibody repertoires with preservation of the natural heavy and light chain pairing. J. Mol Biol., 358, 764-772 (2006).
Morozov, Dao Thi, & Denner, The transmembrane protein of the human endogenous retrovirus—K (HERV-K) modulates cytokine release and gene expression. PLoS One., 8, e70399 (2013).
Natsume, Niwa, & Satoh, Improving effector functions of antibodies for cancer treatment: Enhancing ADCC and CDC. Drug Des. Devel. Ther., 3, 7-16 (2009).
Novak et al., Safety and immunogenicity of the tau vaccine AADvac1 in patients with Alzheimer's disease: A randomised, double-blind, placebo-controlled, phase 1 trial. Lancet Neurol., 16, 123-134 (2017).
Ono, Kawakami, & Ushikubo, Stimulation of expression of the human endogenous retrovirus genome by female steroid hormones in human breast cancer cell line T47D. J. Virol., 61, 2059-2062 (1987).
Ono, Yasunaga, Miyata, & Ushikubo, Nucleotide sequence of human endogenous retrovirus genome related to the mouse mammary tumor virus genome. J. Virol. 60, 589-598 (1986). The most biologically active HERVs are members of the HERV-K family. HERV-K has a complete sequence capable of expressing all the elements needed for a replication-competent retrovirus.
Oricchio et al., Distinct roles for LINE-1 and HERV-K retroelements in cell proliferation, differentiation and tumor progression. Oncogene, 26, 4226-4233 (2007).
Robinson-McCarthy et al., Reconstruction of the cell entry pathway of an extinct virus. PLoS Pathog., 14, e1007123 (2018).
Rycaj et al., Cytotoxicity of human endogenous retrovirus K-specific T cells toward autologous ovarian cancer cells. Clin Cancer Res., 21, 471-483 (2015). The inventors observed the expression of HERVs, especially HERV-K sequences, in breast, lung, prostate, ovarian, colon, pancreatic, and other solid tumors.
Scheid et al., Broad diversity of neutralizing antibodies isolated from memory B cells in HIV-infected individuals. Nature, 458, 636-640 (2009).
Seifarth et al., Retrovirus-like particles released from the human breast cancer cell line T47-D display type B- and C-related endogenous retroviral sequences. J. Virol., 69, 6408-6416 (1995).
Seitz et al., Reconstitution of paired T cell receptor alpha- and beta-chains from microdissected single cells of human inflammatory tissues. Proc. Natl. Acad. Sci. U.S.A., 103, 12057-12062 (2006).
Serafino et al., The activation of human endogenous retrovirus K (HERV-K) is implicated in melanoma cell malignant transformation. Exp. Cell Res., 315, 849-862 (2009).
Smith et al., Rapid generation of fully human monoclonal antibodies specific to a vaccinating antigen. Nature Protoc., 4, 372-384 (2009).
Song & Liu, A TLR9 agonist enhances the anti-tumor immunity of peptide and lipopeptide vaccines via different mechanisms. Science Reports., 5, 12578 (2015).
Spector & Blackwell, Understanding the mechanisms behind trastuzumab therapy for human epidermal growth factor receptor 2-positive breast cancer. J. Clin Oncol., 27, 5838-5847 (2009).
Wallace et al., Elevated HERV-K mRNA expression in PBMC is associated with a prostate cancer diagnosis particularly in older men and smokers. Carcinogenesis., 35, 2074-2083 (2014).
Wang-Johanning et al., Detecting the expression of human endogenous retrovirus E envelope transcripts in human prostate adenocarcinoma. Cancer, 98, 187-197 (2003). The inventors observed the expression of HERVs, especially HERV-K sequences, in breast, lung, prostate, ovarian, colon, pancreatic, and other solid tumors.
Wang-Johanning et al., Expression of human endogenous retrovirus k envelope transcripts in human breast cancer. Clinical Cancer Res. 7, 1553-1560 (2001).
Wang-Johanning et al., Expression of multiple human endogenous retrovirus surface envelope proteins in ovarian cancer. Int. J. Cancer., 120, 81-90 (2007).
Wang-Johanning et al., Human endogenous retrovirus K triggers an antigen-specific immune response in breast cancer patients. Cancer Res., 68, 5869-5877 (2008). The inventors observed the expression of HERVs, especially HERV-K sequences, in breast, lung, prostate, ovarian, colon, pancreatic, and other solid tumors. Wang-Johanning et al., Human endogenous retrovirus type K antibodies and mRNA as serum biomarkers of early-stage breast cancer. Int. J. Cancer., 134, 587-595 (2014).
Wang-Johanning et al., Immunotherapeutic potential of anti-human endogenous retrovirus-K envelope protein antibodies in targeting breast tumors. J. Natl Cancer Inst., 104, 189-210 (2012). The inventors showed that the HERV-K Env protein is commonly expressed on the surface of breast cancer cells. The inventors observed the expression of HERVs, especially HERV-K sequences, in breast, lung, prostate, ovarian, colon, pancreatic, and other solid tumors 30-34.
Wang-Johanning et al., Quantitation of HERV-K env gene expression and splicing in human breast cancer. Oncogene, 22, 1528-1535 (2003). The inventors observed the expression of HERVs, especially HERV-K sequences, in breast, lung, prostate, ovarian, colon, pancreatic, and other solid tumors.
Wang-Johanning et al., Tumor microenvironment predicts aggressive breast cancer: Combination of HERV-K, immune checkpoint and activation status of CD8+ T cells. Cancer Res., 77, Abstract nr LB-221 (2017).
Zahavi, Monoclonal Antibodies in Cancer Therapy. MDPI Web. (Jul. 20, 2020).
Zhang et al., An EpCAM/CD3 bispecific antibody efficiently eliminates hepatocellular carcinoma cells with limited galectin-1 expression. Cancer Immunol. Immunother., 63, 121-132 (2014).
Zhao et al. Expression of Human Endogenous Retrovirus Type K Envelope Protein is a Novel Candidate Prognostic Marker for Human Breast Cancer. Genes Cancer., 2, 914-922 (2011). The inventors observed the expression of HERVs, especially HERV-K sequences, in breast, lung, prostate, ovarian, colon, pancreatic, and other solid tumors.
Zhou et al., Activation of HERV-K Env protein is essential for tumorigenesis and metastasis of breast cancer cells. Oncotarget, 7, 84093-84117 (2016).
Zhou et al., Chimeric antigen receptor T cells targeting HERV-K inhibit breast cancer and its metastasis through downregulation of Ras. Oncoimmunology, 4, e1047582 (2015). The inventors observed the expression of HERVs, especially HERV-K sequences, in breast, lung, prostate, ovarian, colon, pancreatic, and other solid tumors.
Zhou, Zou, Zhang, & Marks, Internalizing cancer antibodies from phage libraries selected on tumor cells and yeast-displayed tumor antigens. J. Mol Biol., 404, 88-99 (2010).

### SEQUENCE LISTING

SEQUENCE LISTING

SEQ ID NO: 1
Sequence of the Mouse ScFV gene VH-Linker-VL
ATGGCCCAGGTGAAGCTGCAGCAGTCAGGACCTGACCTGGTGAAGCCTGGGGCTTCAGTGAAGAT
ATCCTGCAAGGCGTCTGGTTACTCATTCACTGGCTACTACATGCACTGGGTGAAGCAGAGCCATG
GAAAGAGCCTTGAGTGGATTGGACGTGTTAATCCTAACAGTGGTGGTACAAGCTACAACCAGAAG
TTCAAGGACAAGGCCATATTAACTGTAGACAAGTCATCCAGCACAGCCTACATGGAGCTCCGCAG
CCTGACATCTGAGGACTCTGCGGTCTATTACTGTGCAAGATCGAAAGGTAACTACTTCTATGCTA
TGGACTACTGGGGCCAAGGGACCACGGTCACCGTCTCCTCAAGTGGAGGCGGTTCAGGCGGAGGT
GGCTCTGGCGGTGGCGGATCGGACATCGAGCTCACTCAGTCTCCAGCTTCTTTGGCTGTGTCTCT
AGGGCAGAGGGCCACCATATCCTGCAGAGCCAGTGAAAGTGTTGATAGTCATGGCACTAGTTTTA
TGCACTGGTACCAGCAGAAACCAGGACAGCCACCCAAATTCCTCATCTATCGTGCATCCAACCTA
GAATCTGGGATCCCTGCCAGGTTCAGTGGCAGTGGGTCTAGGACAGACTTCACCCTCACCATTAA
TCCTGTGGAGACAGATGATGTTGCAATCTATTACTGTCAGCAAAGTAATGAGGATCCTCCGACGT
TCGGTGGAGGCACCAAGCTGGAAATCAAAC
SEQ ID NO: 2
Sequence of the Mouse ScFV gene: VH
CAGGTGAAGCTGCAGCAGTCAGGACCTGACCTGGTGAAGCCTGGGGCTTCAGTGAAGATATCCTG
CAAGGCGTCTGGTTACTCATTCACTGGCTACTACATGCACTGGGTGAAGCAGAGCCATGGAAAGA
GCCTTGAGTGGATTGGACGTGTTAATCCTAACAGTGGTGGTACAAGCTACAACCAGAAGTTCAAG
GACAAGGCCATATTAACTGTAGACAAGTCATCCAGCACAGCCTACATGGAGCTCCGCAGCCTGAC
ATCTGAGGACTCTGCGGTCTATTACTGTGCAAGATCGAAAGGTAACTACTTCTATGCTATGGACT
ACTGGGGCCAAGGGACCACGGTCACCGTCTCCTCAA
SEQ ID NO: 3
Sequence of the Mouse ScFV gene: >FWJ_VH
CDRs are highlighted
QVKLQQSGPDLVKPGASVKISCKASGYSFTGYYMHWVKQSHGKSLEWIGRVNPNSGGTSYNQKFK
DKAILTVDKSSSTAYMELRSLTSEDSAVYYCARSKGNYFYAMDYWGQGTTVTVSS
SEQ ID NO: 4
Sequence of the Mouse ScFV gene: VL
GACATCGAGCTCACTCAGTCTCCAGCTTCTTTGGCTGTGTCTCTAGGGCAGAGGGCCACCATATC
CTGCAGAGCCAGTGAAAGTGTTGATAGTCATGGCACTAGTTTTATGCACTGGTACCAGCAGAAAC
CAGGACAGCCACCCAAATTCCTCATCTATCGTGCATCCAACCTAGAATCTGGGATCCCTGCCAGG
TTCAGTGGCAGTGGGTCTAGGACAGACTTCACCCTCACCATTAATCCTGTGGAGACAGATGATGT
TGCAATCTATTACTGTCAGCAAAGTAATGAGGATCCTCCGACGTTCGGTGGAGGCACCAAGCTGG
AAATCAAAC
SEQ ID NO: 5
Sequence of the Mouse ScFV gene: >FWJ_VL
CDRs are highlighted
DIELTQSPASLAVSLGQRATISCRASESVDSHGTSFMHWYQQKPGQPPKFLIYRASNLESGIPAR
FSGSGSRTDFTLTINPVETDDVAIYYCQQSNEDPPTFGGGTKLEIK
SEQ ID NO: 6
Sequence of the Humanized ScFV gene: >HUM1-FWJVH
EVQLVESGGGLVQPGGSLRLSCKASGYSFTGYYMHWVRQAPGKGLEWIGRVNPNSGGTSYNQKFK
DRATLSVDNSKNTAYLQMNSLRAEDTAVYYCARSKGNYFYAMDYWGQGTLVTVSS
SEQ ID NO: 7
Sequence of the Humanized ScFV gene: >HUM2 FWJ_VH
EVQLVESGGGLVQPGGSLKVSCKASGYSFTGYYMHWVRQASGKGLEWIGRVNPNSGGTSYNQKFK
DRFTISRDKSISTLYLQMSSLRSEDTAVYYCARSKGNYFYAMDYWGQGTLVTVSS
SEQ ID NO: 8
Sequence of the Mouse ScFV gene: >FWJ_VH
QVKLQQSGPDLVKPGASVKISCKASGYSFTGYYMHWVKQSHGKSLEWIGRVNPNSGGTSYNQKFK
DKAILTVDKSSSTAYMELRSLTSEDSAVYYCARSKGNYFYAMDYWGQGTTVTVSS
SEQ ID NO: 9
Sequence of the Humanized ScFV gene: >HUM1_FWJ_VL
DIQMTQSPSSLSASVGDRVTITCRASESVDSHGTSFMHWYQQKPGKAPKFLIYRASNLESGIPSR
FSGSGSGTDFTLTISSVQPEDFAVYYCQQSNEDPPTFGGGTKVEIK
SEQ ID NO: 10
Sequence of the Humanized ScFV gene: >HUM2_FWJ_VL
DIQMTQSPSSLSASVGDRVTISCRASESVDSHGTSFMHWYQQKPGKSPKFLIYRASNLESGIPSR
FSGSGSGTDFTLTISSLQPEDFAIYYCQQSNEDPPTFGGGTKVEIK
SEQ ID NO: 11
Sequence of the Mouse ScFV gene: >FWJ_VL
DIELTQSPASLAVSLGQRATISCRASESVDSHGTSFMHWYQQKPGQPPKFLIYRASNLESGIPAR
FSGSGSRTDFTLTINPVETDDVAIYYCQQSNEDPPTFGGGTKLEIK
SEQ ID NO: 12
Final humanized version of scFv gene scFv-1
EVQLVESGGGLVQPGGSLRLSCKASGYSFTGYYMHWVRQAPGKGLEWIGRVNPNSGGTSYNQKFK
DRATLSVDNSKNTAYLQMNSLRAEDTAVYYCARSKGNYFYAMDYWGQGTLVTVSSGGGGSGGGGS
GGGGSDIQMTQSPSSLSASVGDRVTITCRASESVDSHGTSFMHWYQQKPGKAPKFLIYRASNLES
GIPSRFSGSGSGTDFTLTISSVQPEDFAVYYCQQSNEDPPTFGGGTKVEIK
SEQ ID NO: 13
Final humanized version of scFv gene scFv-2
EVQLVESGGGLVQPGGSLKVSCKASGYSFTGYYMHWVRQASGKGLEWIGRVNPNSGGTSYNQKFK
DRFTISRDKSISTLYLQMSSLRSEDTAVYYCARSKGNYFYAMDYWGQGTLVTVSSGGGGSGGGGS
GGGGSDIQMTQSPSSLSASVGDRVTISCRASESVDSHGTSFMHWYQQKPGKSPKFLIYRASNLES
GIPSRFSGSGSGTDFTLTISSLQPEDFAIYYCQQSNEDPPTFGGGTKVEIK
SEQ ID NO: 14
Standard 17 amino acid linker
SSGGGGSGGGGSGGGGS
SEQ ID NO: 15
Final humanized version of scFv gene: Humanized scFv-1
FWJ_HUMscFv-1
EVQLVESGGGLVQPGGSLRLSCKASGYSFTGYYMHWVRQAPGKGLEWIGRVNPNSGGTSYNQKFK
DRATLSVDNSKNTAYLQMNSLRAEDTAVYYCARSKGNYFYAMDYWGQGTLVTVSSGGGGSGGGGS
GGGGSDIQMTQSPSSLSASVGDRVTITCRASESVDSHGTSFMHWYQQKPGKAPKFLIYRASNLES
GIPSRFSGSGSGTDFTLTISSVQPEDFAVYYCQQSNEDPPTFGGGTKVEIK
SEQ ID NO: 16
Final humanized version of scFv gene: > Humanized scFv-
1 polynucleotide (HUM1)
GAGGTGCAGCTGGTGGAGAGCGGCGGCGGCCTGGTGCAGCCCGGCGGCAGCCTGAGGCTGAGCTG
CAAGGCCAGCGGCTACAGCTTCACCGGCTACTACATGCACTGGGTGAGGCAGGCCCCCGGCAAGG
GCCTGGAGTGGATCGGCAGGGTGAACCCCAACAGCGGCGGCACCAGCTACAACCAGAAGTTCAAG
GACAGGGCCACCCTGAGCGTGGACAACAGCAAGAACACCGCCTACCTGCAGATGAACAGCCTGAG
GGCCGAGGACACCGCCGTGTACTACTGCGCCAGGAGCAAGGGCAACTACTTCTACGCCATGGACT
ACTGGGGCCAGGGCACCCTGGTGACCGTGAGCAGCGGCGGCGGCGGCAGCGGCGGCGGCGGCAGC
GGCGGCGGCGGCAGCGACATCCAGATGACCCAGAGCCCCAGCAGCCTGAGCGCCAGCGTGGGCGA
CAGGGTGACCATCACCTGCAGGGCCAGCGAGAGCGTGGACAGCCACGGCACCAGCTTCATGCACT
GGTACCAGCAGAAGCCCGGCAAGGCCCCCAAGTTCCTGATCTACAGGGCCAGCAACCTGGAGAGC
GGCATCCCCAGCAGGTTCAGCGGCAGCGGCAGCGGCACCGACTTCACCCTGACCATCAGCAGCGT
GCAGCCCGAGGACTTCGCCGTGTACTACTGCCAGCAGAGCAACGAGGACCCCCCCACCTTCGGCG
GCGGCACCAAGGTGGAGATCAAG
SEQ ID NO: 18
Final humanized version of scFv gene: Humanized FWJ_HUMscFv-2
EVQLVESGGGLVQPGGSLKVSCKASGYSFTGYYMHWVRQASGKGLEWIGRVNPNSGGTSYNQKFK
DRFTISRDKSISTLYLQMSSLRSEDTAVYYCARSKGNYFYAMDYWGQGTLVTVSSGGGGSGGGGS
GGGGSDIQMTQSPSSLSASVGDRVTISCRASESVDSHGTSFMHWYQQKPGKSPKFLIYRASNLES
GIPSRFSGSGSGTDFTLTISSLQPEDFAIYYCQQSNEDPPTFGGGTKVEIK
SEQ ID NO: 19
Final humanized version of scFv gene: >> Humanized scFv-
2_polypeptide
GAGGTGCAGCTGGTGGAGAGCGGCGGCGGCCTGGTGCAGCCCGGCGGCAGCCTGAAGGTGAGCTG
CAAGGCCAGCGGCTACAGCTTCACCGGCTACTACATGCACTGGGTGAGGCAGGCCAGCGGCAAGG
GCCTGGAGTGGATCGGCAGGGTGAACCCCAACAGCGGCGGCACCAGCTACAACCAGAAGTTCAAG
GACAGGTTCACCATCAGCAGGGACAAGAGCATCAGCACCCIGTACCTGCAGATGAGCAGCCTGAG
GAGCGAGGACACCGCCGTGTACTACTGCGCCAGGAGCAAGGGCAACTACTICTACGCCATGGACT
ACTGGGGCCAGGGCACCCTGGTGACCGTGAGCAGCGGGGGGGGCGGCAGCGGCGGGGGGGGCAGC
GGCGGCGGCGGCAGCGACATCCAGATGACCCAGAGCCCCAGCAGCCTGAGCGCCAGCGTGGGCGA
CAGGGTGACCATCAGCTGCAGGGCCAGCGAGAGCGTGGACAGCCACGGCACCAGCTTCATGCACT
GGTACCAGCAGAAGCCCGGCAAGAGCCCCAAGTTCCTGATCTACAGGGCCAGCAACCTGGAGAGC
GGCATCCCCAGCAGGTTCAGCGGCAGCGGCAGCGGCACCGACTTCACCCTGACCATCAGCAGCCT
GCAGCCCGAGGACTTCGCCATCTACTACTGCCAGCAGAGCAACGAGGACCCCCCCACCTTCGGCG
GCGGCACCAAGGTGGAGATCAAG
SEQ ID NO: 20
OKT8 Heavy chains: (Anti-CD8 mAb sequence):
tttgaggtccagctgcagcagtctggggcagagcttgtgaagccaggggcctcagtcaagttgtc
ctgcacagcttctggettcaacattaaagacacctatatacacttcgtgaggcagaggcctgaac
agggcctggagtggattggaaggattgatcctgcgaatgataatactttatatgcctcaaagttc
cagggcaaggccactataacagcagacacatcatccaacacagcctacatgcacctctgcagcct
gacatctggggacactgccgtctattactgtggtagaggttatggttactacgtatttgaccact
tgggccaaggc
SEQ ID NO: 21
OKT8 Heavy chains: (Anti-CD8 mAb sequence): >H1
CDRs are highlighted
FEVQLQQSGAELVKPGASVKLSCTASGFNIKDTYIHFVRQRPEQGLEWIGRIDPANDNTLYASKF
QGKATITADTSSNTAYMHLCSLISGDTAVYYCGRGYGYYVFDHLGQG
SEQ ID NO: 22
OKT8 Heavy chains: (Anti-CD8 mAb sequence): >H3
tntcaggtgcagctgaagcagtctggggcagagcttgtgaagccaggggcctcagtcaagttgtc
ctgcacagcttctggcttcaacattaaagacacctatatacacttcgtgaggcagaggcctgaac
agggcctggagtggattggaaggattgatcctgcgaatgataatactttatatgcctcaaagttc
cagggcaaggccactataacagcagacacatcatccaacacagcctacatgcacctctgcagcct
gacatctggggacactgccgtctattactgtggtagaggttatggttactacgtatttgaccact
ggggccaaggcaccactctcaca
SEQ ID NO: 23
OKT8 Heavy chains: (Anti-CD8 mAb sequence): >H3
CDRs are highlighted
XQVQLKQSGAELVKPGASVKLSCTASGFNIKDTYIHFVRQRPEQGLEWIGRIDPANDNTLYASKF
QGKATITADTSSNTAYMHLCSLTSGDTAVYYCGRGYGYYVFDHWGQGTTLT
SEQ ID NO: 24
OKT8 Heavy chains: (Anti-CD8 mAb sequence): >H6
tttgaggtccagctgcagcagtctggggcagagcttgtgaagccaggggcctcagtcaagttgtc
ctgcacagcttctggcttcaacattaaagacacctatatacacttcgtgaggcagaggcctgaac
agggcctggagtggattggaaggattgatcctgcgaatgataatactttatatgcctcaaagttc
cagggcaaggccactataacagcagacacatcatccaacacagcctacatgcacctctgcagcct
gacatctggggacactgccgtctattactgtggtagaggttatggttactacgtatttgaccact
ggggccaaggcaccactctcaca
SEQ ID NO: 25
OKT8 Heavy chains: (Anti-CD8 mAb sequence): >H6
CDRs are highlighted
FEVQLQQSGAELVKPGASVKLSCTASGFNIKDTYIHFVRQRPEQGLEWIGRIDPANDNTLYASKF
QGKATITADTSSNTAYMHLCSLTSGDTAVYYCGRGYGYYVFDHWGQGTTLT
SEQ ID NO: 26
OKT8 Heavy chains: (Anti-CD8 mAb sequence): >H7
tcaggtccaactgcagcagcctggggcagagcttgtgaagccaggggcctcagtcaagttgtcct
gcacagcttctggcttcaacattaaagacacctatatacacttcgtgaggcagaggcctgaacag
ggcctggagtggattggaaggattgatcctgcgaatgataatactttatatgcctcaaagttcca
gggcaaggccactataacagcagacacatcatccaacacagcctacatgcacctctgcagcctga
catctggggacactgccgtctattactgtggtagaggttatggttactacgtatttgaccactgg
ggccaaggcaccactctcacat
SEQ ID NO: 27
OKT8 Heavy chains: (Anti-CD8 mAb sequence): >H7
CDRs are highlighted
QVQLQQPGAELVKPGASVKLSCTASGFNIKDTYIHFVRQRPEQGLEWIGRIDPANDNTLYASKFQ
GKATITADISSNTAYMHLCSLTSGDTAVYYCGRGYGYYVFDHWGQGTTLT
SEQ ID NO: 28
OKT8 Light chains: (Anti-CD8 mAb sequence): >K4
GACATTGTGCTGACCCAATCTCCTGCTTCCTTAGCTGTATCTCTGGGGCAGAGGGCCACCATCT
CATACAGGGCCAGCAAAAGTGTCAGTACATCTGGCTATAGTTATATGCACTGGAACCAACAGAA
ACCAGGACAGCCACCCAGACTCCTCATCTATCTTGTATCCAACCTAGAATCTGGGGTCCCTGCC
AGGTTCAGTGGCAGTGGGTCTGGGACAGACTTCACCCTCAACATCCATCCTGTGGAGGAGGAGG
ATGCTGCAACCTATTACTGTCAGCACATTAGGGAGCTTACACGTTCGGAGGGGGACCAAGC
SEQ ID NO: 29
OKT8 Light chains: (Anti-CD8 mAb sequence): >K4
CDRs are highlighted
DIVLTQSPASLAVSLGQRATISYRASKSVSTSGYSYMHWNQQKPGQPPRLLIYLVSNLESGVPAR
FSGSGSGTDFTLNIHPVEEEDAATYYCQHIRELTRSEGDQ
SEQ ID NO: 30
OKT8 Light chains: (Anti-CD8 mAb sequence): >K6
GATATTGTGCTAACTCAGTCTCCTGCTTCCTTAGCTGTATCTCTGGGGCAGAGGGCCACCATCT
CATACAGGGCCAGCAAAAGTGTCAGTACATCTGGCTATAGTTATATGCACTGGAACCAACAGAA
ACCAGGACAGCCACCCAGACTCCTCATCTATCTTGTATCCAACCTAGAATCTGGGGTCCCTGCC
AGGTTCAGTGGCAGTGGGTCTGGGACAGACTTCACCCTCAACATCCATCCTGTGGAGGAGGAGG
ATGCTGCAACCTATTACTGTCAGCACATTAGGGAGCTTACACG
SEQ ID NO: 31
OKT8 Light chains: (Anti-CD8 mAb sequence): >K6
CDRs are highlighted
DIVLTQSPASLAVSLGQRATISYRASKSVSTSGYSYMHWNQQKPGQPPRLLIYLVSNLESGVPAR
FSGSGSGTDFTLNIHPVEEEDAATYYCQHIRELT
SEQ ID NO: 32
OKT8 Light chains: (Anti-CD8 mAb sequence): >K8
GACATCCAGCTGACTCAGTCTCCTGCTTCCTTAGCTGTATCTCTGGGGCAGAGGGCCACCATCT
CATACAGGGCCAGCAAAAGTGTCAGTACATCTGGCTATAGTTATATGCACTGGAACCAACAGAA
ACCAGGACAGCCACCCAGACTCCTCATCTATCTTGTATCCAACCTAGAATCTGGGGTCCCTGCC
AGGTTCAGTGGCAGTGGGTCTGGGACAGACTTCACCCTCAACATCCATCCTGTGGAGGAGGAGG
ATGCTGCTGACCTATTAGCTGTCAGCACATTATGGGAGCTTACACGTTCGG
SEQ ID NO: 33
OKT8 Light chains: (Anti-CD8 mAb sequence): >K8
CDRs are highlighted
DIQLTQSPASLAVSLGQRATISYRASKSVSTSGYSYMHWNQQKPGQPPRLLIYLVSNLESGVPAR
FSGSGSGTDFTLNIHPVEEEDAADLLAVSTLWELTRS
SEQ ID NO: 34
CD8 BITE
acc ggt atg gat atc gag ctg acc cag agc cct agc agc ctg gcc gtg
tca ctg ggc cag aga gcc acc atc agc tgc aga gcc tcc gag agc gtg
gat agc cac ggc acc agc ctg atg cac tgg tat cag cag aag ccc ggc
cag ccc ccc aag ttc ctg atc tac cgg gcc agc aac ctg gaa agc ggc
atc ccc gcc aga ttt tcc ggc agc ggc agc aga acc gac ttc acc ctg
acc atc aac ccc gtg gag aca gac gac gtg gcc atc tac tac tgc cag
cag agc aac gag gac cct ccc acc ttt ggc gga ggc acc aag ctg gaa
ctg aag gag ggc gga gga agc gga ggg gga gga tct ggc gga ggc ggc
agc gcc cag gtg aag ctg cag cag agc ggc cct gat ctg gtg aag cct
ggc gcc agc gtg aag atc agc tgc aag gcc agc ggc tac agc ttc acc
ggc tac tac atg cac tgg gtg aaa cag agc cac ggc aag agc ctg gaa
tgg atc ggc aga gtg aac ccc aat agc ggc ggc acc agc tac aac cag
aag ttc aag gac aag gcc atc ctg acc gtg gac aag agc agc agc acc
gcc tac atg gaa ctg cgg agc ctg acc agc gag gac agc gcc gtg tac
tac tgc gcc cgg tcc aag ggc aac tac ttc tac gcc atg gac tac tgg
ggc cag ggc acc acc gtg acc gtg tct agc agc ggc ggc ggc gga agc
gaa gtg cag ctg cag cag tcc gga gca gaa ctg gtg aaa ccc gga gcc
agt gtg aag ctg agc tgt aca gcc tcc ggc ttt aat atc aag gac acc
tac atc cac ttc gtg cgc caa cgg cca gaa cag ggt ctg gag tgg att
ggc agg atc gat cca gca aat gat aac acc ctg tac gca agc aaa ttt
cag ggc aaa gcc acg ata acc gcc gat aca tct agt aat acg gct tac
atg cac ctc tgc tcc ctg act tcc ggg gac acc gcc gtg tat tat tgc
ggg cgc gga tac ggt tac gtg ttc gat cat tgg ggt cag ggc acc ctc
aca gtc tcc agt gca gga ggc gga ggt agt gga ggc gga ggc tct ggc
ggt ggc ggg tca gac ata gtg ctg aca cag tct ccc gcc agt ctg gca
gtg agc ctt ggc cag agg gct aca att tcc tat agg gcg tct aaa tcc
gtt agc act tca ggt tac tct tat atg cac tgg aac cag aag cct ggt
cag ccc agg ctt ctt att tac ctg gtc agc aat ctc gag tct ggc gtg
ccc gct aga ttt tcc ggc agc ggg agt ggg act gac ttc act ctg aac
atc cac cca gtg gag gaa gag gat gcc gca acc tac tat tgt cag cat
atc cgg gaa ttg acc aga tca gag ggc gga cct tcc tgg aag gag cag
aag ctg att agc gag gaa gat ctg gat tat aag gac gat gac aaa tga
gaa ttc
SEQ ID NO: 35
CD8 BITE
TGMDIELTQSPSSLAVSLGQRATISCRASESVDSHGTSLMHWYQQKPGQPPKFLIYRASNLESGI
PARFSGSGSRTDFTLTINPVETDDVAIYYCQQSNEDPPTFGGGTKLELKEGGGSGGGGSGGGGSA
QVKLQQSGPDLVKPGASVKISCKASGYSFTGYYMHWVKQSHGKSLEWIGRVNPNSGGTSYNQKFK
DKAILTVDKSSSTAYMELRSLTSEDSAVYYCARSKGNYFYAMDYWGQGTTVTVSSSGGGGSEVQL
QQSGAELVKPGASVKLSCTASGFNIKDTYIHFVRQRPEOGLEWIGRIDPANDNTLYASKFQGKAT
ITADTSSNTAYMHLCSLISGDTAVYYCGRGYGYYVFDHWGQGTTLTVSSAGGGGSGGGGSGGGGS
DIVLTQSPASLAVSLGQRATISYRASKSVSTSGYSYMHWNQQKPGQPPRLLIYLVSNLESGVPAR
FSGSGSGTDFTLNIHPVEEEDAATYYCQHIRELTRSEGGPSWKEQKLISEEDLDYKDDDDK-EF
SEQ ID NO: 36
CD3 BITE
acc ggt atg gat atc gag ctg acc cag agc cct agc ctg gcc gtg tca
ctg ggc cag aga gcc acc atc agc tgc aga gcc tcc gag agc gtg gat
agc cac ggc acc agc ctg atg cac tgg tat cag cag aag ccc ggc cag
ccc ccc aag ttc ctg atc tac cgg gcc agc aac ctg gaa agc ggc atc
ccc gcc aga ttt tcc ggc agc ggc agc aga acc gac ttc acc ctg acc
atc aac ccc gtg gag aca gac gac gtg gcc atc tac tac tgc cag cag
agc aac gag gac cct ccc acc ttt ggc gga ggc acc aag ctg gaa ctg
aag gag ggc gga gga agc gga ggg gga gga tct ggc gga ggc ggc agc
gcc cag gtg aag ctg cag cag agc ggc cct gat ctg gtg aag cct ggc
gcc agc gtg aag atc agc tgc aag gcc agc ggc tac agc ttc acc ggc
tac tac atg cac tgg gtg aaa cag agc cac ggc aag agc ctg gaa tgg
atc ggc aga gtg aac ccc aat agc ggc ggc acc agc tac aac cag aag
ttc aag gac aag gcc atc ctg acc gtg gac aag agc agc agc acc gcc
tac atg gaa ctg cgg agc ctg acc agc gag gac agc gcc gtg tac tac
tgc gcc cgg tcc aag ggc aac tac ttc tac gcc atg gac tac tgg ggc
cag ggc acc acc gtg acc gtg tct agc agc ggc ggc ggc gga agc gag
gtt cag ctg gtg gag tct ggc ggt ggc ctg gtg cag cca ggg ggc tca
ctc cgt ttg tcc tgt gca gct tct ggc tac tcc ttt acc ggc tac act
atg aac tgg gtg cgt cag gcc cca ggt aag ggc ctg gaa tgg gtt gca
ctg att aat cct tat aaa ggt gtt tcc acc tat aac cag aaa ttc aag
gat cgt ttc acg ata tcc gta gat aaa tcc aaa aac aca gcc tac ctg
caa atg aac agc ctg cgt gct gag gac act gcc gtc tat tat tgt gct
aga agc gga tac tac ggc gat agc gac tgg tat ttt gac gtc tgg ggt
caa gga acc ctg gtc acc gtc tcc tcg ggt gga ggc ggt tca ggc gga
ggt ggc tct ggc ggt ggc gga tcg gat atc cag atg acc cag tcc ccg
agc tcc ctg tcc gcc tct gtg ggc gat agg gtc acc atc acc tgt cgt
gcc agt cag gac atc cgt aat tat ctc aac tgg tat caa cag aaa cca
gga aaa gct ccg aaa cta ctg att tac tat acc tcc cgc ctg gag tct
gga gtc cct tct cgc ttc tct ggt tct ggt tct ggg acg gat tac act
ctg acc atc agc agt ctg caa ccg gag gac ttc gca act tat tac tgt
cag caa ggt aat act ctg ccg tgg acg ttc gga cag ggc acc aag gtg
gag atc aaa gag cag aag ctg att agc gag gaa gat ctg gat tat aag
gac gac gat gac aaa tga gaa ttc
SEQ ID NO: 37
CD3 BITE
TGMDIELTQSPSSLAVSLGQRATISCRASESVDSHGTSLMHWYQQKPGQPPKFLIYRASNLESGI
PARFSGSGSRIDFTLTINPVETDDVAIYYCQQSNEDPPTFGGGTKLELKEGGGSGGGGSGGGGSA
QVKLQQSGPDLVKPGASVKISCKASGYSFTGYYMHWVKQSHGKSLEWIGRVNPNSGGISYNQKFK
DKAILTVDKSSSTAYMELRSLTSEDSAVYYCARSKGNYFYAMDYWGQGTTVTVSSSGGGGSEVQL
VESGGGLVQPGGSLRLSCAASGYSFTGYTMNWVRQAPGKGLEWVALINPYKGVSTYNQKFKDRFT
ISVDKSKNTAYLQMNSLRAEDTAVYYCARSGYYGDSDWYFDVWGQGTLVTVSSGGGGSGGGGSGG
GGSDIQMTQSPSSLSASVGDRVTITCRASQDIRNYLNWYQQKPGKAPKLLIYYTSRLESGVPSRF
SGSGSGTDYTLTISSLQPEDFATYYCQQGNTLPWTFGQGTKVEIKEQKLISEEDLDYKDDDDK-
EF
SEQ ID NO: 38
FLAG-tag
DYKDDDDK
SEQ ID NO: 39
Myc-tag
EQKLISEEDL
SEQ ID NO: 40
Heavy chain sequence
Color scheme: Signal peptide - VH - CH (Human IgG1)
TGATTCTAGA is a stop codon
GCGGCCGCAAACTACAAGACAGACTTGCAAAAGAAGGCATGCACAGCTCAGCACTGCTCTGTTGC
CTGGTCCTCCTGACTGGGGTGAGGGCCGAGGTGCAGCTGGTGGAGAGCGGGGGGGGACTGGTGCA
GCCAGGAGGAAGCCTGAGACTGAGCTGTAAGGCTTCAGGATACAGCTTCACAGGGTACTATATGC
ACTGGGTGAGACAGGCCCCCGGAAAAGGACTGGAGTGGATCGGCAGAGTGAACCCCAACAGCGGA
GGCACCAGCTACAACCAGAAATTCAAGGACCGGGCCACCCTGAGCGTGGACAACAGCAAAAACAC
AGCCTACCTGCAGATGAACAGCCTGCGGGCCGAGGACACCGCCGTGTACTATTGTGCCAGAAGCA
AGGGCAACTACTTCTACGCCATGGACTATTGGGGCCAGGGAACACTGGTGACCGTGAGCAGCGCT
AGCACCAAGGGCCCATCGGTCTTCCCCCTGGCACCCTCCTCCAAGAGCACCTCTGGGGGCACAGC
GGCCCTGGGCTGCCTGGTCAAGGACTACTTCCCCGAACCGGTGACGGTGTCGTGGAACTCAGGCG
CCCTGACCAGCGGCGTGCACACCTTCCCGGCTGTCCTACAGTCCTCAGGACTCTACTCCCTCAGC
AGCGTGGTGACCGTGCCCTCCAGCAGCTTGGGCACCCAGACCTACATCTGCAACGTGAATCACAA
GCCCAGCAACACCAAGGTGGACAAGAAAGTTGAGCCCAAATCTTGTGACAAAACTCACACATGCC
CACCGTGCCCAGCACCTGAACTCCTGGGGGGACCGTCAGTCTTCCTCTTCCCCCCAAAACCCAAG
GACACCCTCATGATCTCCCGGACCCCCGAGGTCACATGCGTGGTGGTGGACGTGAGCCACGAAGA
CCCTGAGGTCAAGTTCAACTGGTACGTGGACGGCGTGGAGGTGCATAATGCCAAGACAAAGCCGC
GGGAGGAGCAGTACAACAGCACGTACCGTGTGGTCAGCGTCCTCACCGTCCTGCACCAGGACTGG
CTGAATGGCAAGGAGTACAAGTGCAAGGTCTCCAACAAAGCCCTCCCAGCCCCCATCGAGAAAAC
CATCTCCAAAGCCAAAGGGCAGCCCCGAGAACCACAGGTGTACACCCTGCCCCCATCCCGGGAGG
AGATGACCAAGAACCAGGTCAGCCTGACCTGCCTGGTCAAAGGCTTCTATCCCAGCGACATCGCC
GTGGAGTGGGAGAGCAATGGGCAGCCGGAGAACAACTACAAGACCACGCCTCCCGTGCTGGACTC
CGACGGCICCTTCTTCCTCTACAGCAAGCTCACCGTGGACAAGAGCAGGTGGCAGCAGGGGAACG
TCTTCTCATGCTCCGTGATGCATGAGGCTCTGCACAACCACTACACGCAGAAGAGCCTCTCCCTG
TCTCCGGGTAA
SEQ ID NO: 41
Amino acid sequence:
EVQLVESGGGLVQPGGSLRLSCKASGYSFTGYYMHWVRQAPGKGLEWIGRVNPNSGGTSYNQKFK
DRATLSVDNSKNTAYLQMNSLRAEDTAVYYCARSKGNYFYAMDYWGQGTLVTVSSASTKGPSVFP
LAPSSKSTSGGTAALGCLVKDYFPEPVTVSWNSGALTSGVHTFPAVLQSSGLYSLSSVVTVPSSS
LGTQTYICNVNHKPSNTKVDKKVEPKSCDKTHTCPPCPAPELLGGPSVFLFPPKPKDTLMISRTP
EVTCVVVDVSHEDPEVKFNWYVDGVEVHNAKTKPREEQYNSTYRVVSVLTVLHQDWLNGKEYKCK
VSNKALPAPIEKTISKAKGQPREPQVYTLPPSREEMTKNQVSLTCLVKGFYPSDIAVEWESNGQP
ENNYKTTPPVLDSDGSFFLYSKLTVDKSRWQQGNVFSCSVMHEALHNHYTOKSLSLSPGK
SEQ ID NO: 42
Light chain sequence
Color scheme: Signal peptide - VL- CL (Human Kappa)
TGATTCTAGA is a stop codon
GCGGCCGCAAACTACAAGACAGACTTGCAAAAGAAGGCATGCACAGCTCAGCACTGCTCTGTTGC
CTGGTCCTCCTGACTGGGGTGAGGGCCGACATTCAGATGACCCAGAGCCCCAGCAGCCTGAGCGC
CAGCGTGGGAGACAGAGTGACCATCACCTGCAGAGCCAGCGAGAGCGTGGACAGCCATGGCACCT
CCTTTATGCACTGGTACCAGCAGAAGCCCGGCAAGGCCCCCAAATTCCTGATTTACAGAGCCAGC
AACCTGGAGAGCGGCATCCCCTCAAGATTCTCAGGCAGCGGAAGCGGAACCGACTTCACACTGAC
TATCAGCAGCGTGCAGCCCGAAGATTTTGCTGTGTACTACTGCCAGCAGAGCAACGAAGATCCTC
CTACCTTCGGGGGCGGCACTAAGGTGGAGATCAAGCGTACGGTGGCTGCACCATCTGTCTTCATC
TTCCCGCCATCTGATGAGCAGTTGAAATCTGGAACTGCCTCTGTTGTGTGCCTGCTGAATAACTT
CTATCCCAGAGAGGCCAAAGTACAGTGGAAGGTGGATAACGCCCTCCAATCGGGTAACTCCCAGG
AGAGTGTCACAGAGCAGGACAGCAAGGACAGCACCTACAGCCTCAGCAGCACCCTGACGCTGAGC
AAAGCAGACTACGAGAAACACAAAGTCTACGCCTGCGAAGTCACCCATCAGGGCCTGAGTTCGCC
CGTCACAAAGAGCTTCAACAGGGGAGAGTGTTGATTCTAGA
SEQ ID NO: 43
Amino acid sequence
DIQMTQSPSSLSASVGDRVTITCRASESVDSHGTSFMHWYQQKPGKAPKFLIYRASNLESGIPSR
FSGSGSGTDFTLTISSVQPEDFAVYYCQQSNEDPPTFGGGTKVEIKRTVAAPSVFIFPPSDEQLK
SGTASVVCLLNNFYPREAKVQWKVDNALQSGNSQESVTEQDSKDSTYSLSSTLTLSKADYEKHKV
YACEVTHQGLSSPVIKSENRGEC
SEQ ID NO: 44
scFv against MAPs of HERV-K (sequence for anti-HERV-K mAb)
ATGGCCCAGGTGCAACTGCAGCAGTCAGGGGCTGAGCTTGTGAAGCCTGGGGCTTCAGTGAAGAT
GTCCTGCAAGGCTTCTGGCTACACCTTCACCAGCTACTGGATAACCTGGGTGAAGCAGAGGCCTG
GACAAGGCCTTGAGTGGATTGGAGATATTTATCCTGGTAGTGGTAGTACTAACTACAATGAGAAG
TTCAAGAGCAAGGCCACACTGACTGTAGACACATCCTCCAGCACAGCCTACATGCAGCTCAGCAG
CCTGACATCTGAGGACTCTGCGGTCTATTACTGTGCAAGATGGCGGGACGGGTACTATGCTATGG
ACTACTGGGGCCAAGGGACCACGGTCACCGTCTCCTCAAGTGAAGGCGGTTCAGGCGGAGGTTGC
TCTGGCGGTGGCGGATCGGACATCGAGCTCACTCAGTCTCCAACCACCATGGCTGCATCTCCCGG
GGAGAAGATCACTATCACCTGCAGTGCCAGCTCAAGTATAAGTTCCAATTACTTGCATTGGTATC
AGCAGAAGCCAGGATTCTCCCCTAAACTCTTGATTTATAGGACATCCAATCTGGCTTCTGGAGTC
CCAGCTCGCTTCAGTGGCAGTGGGTCTGGGACCTCTTACTCTCTCACAATTGGCACCATGGAAGC
TGAAGATGTTGCCACTTACTACTGCCAGCAGGGTAGTAGTATACCATTCACGTTCGGCTCGGGGA
CGAAGTTGGAGCTGAAACGGGCGGGCCGCAGGTGCGCCGGTGCCGTATCCG
SEQ ID NO: 45
scFv against MAPs of HERV-K (sequence for anti-HERV-K mAb)
MAQVQLQQSGAELVKPGASVKMSCKASGYTFTSYWITWVKQRPGQGLEWIGDIYPGSGSTNYNEK
FKSKATLTVDTSSSTAYMQLSSLTSEDSAVYYCARWRDGYYAMDYWGQGTTVTVSSSEGGSGGGC
SGGGGSDIELTQSPTTMAASPGEKITITCSASSSISSNYLHWYQQKPGFSPKLLIYRTSNLASGV
PARFSGSGSGTSYSLTIGTMEAEDVATYYCQQGSSIPFTFGSGTKLELKRAGRRCAGAVS
SEQ ID NO: 46
Human germline sequences near the boundary for CDRs in humanized
anti-HERV-K antibodies
VRQAPGKGLEW
SEQ ID NO: 47
Human germline sequences near the boundary for CDRs in humanized
anti-HERV-K antibodies
LQMNSLRAEDTAVYYC
SEQ ID NO: 48
Humanized CD8 heavy chain
CDRs are highlighted
GAGGTCCAGCTGGTACAGTCTGGGGCTGAGGTGAAGAAGCCTGGGGCTACAGTGAAAATCTCCTG
CAAGGTTTCIGGCTTCAACATTAAAGACACCTATATGCACTGGGTGCAACAGGCCCCTGGAAAAG
GGCTTGAGTGGATGGGAAGGATTGATCCTGCGAATGATAATACTTTATACGCAGAGAAGTTCCAG
GGCAGAGTCACCATAACCGCGGACACGTCTACAGACACAGCCTACATGGAGCTGAGCAGCCTGAG
ATCTGAGGACACGGCCGTGTATTACTGTGGTAGAGGTTATGGTTACTACGTATTTGACCACTGGG
GCCAAGGCACC
SEQ ID NO: 49
Humanized CD8 heavy chain
CDRs are highlighted
EVQLVQSGAEVKKPGATVKISCKVSGFNIKDTYMHWVQQAPGKGLEWMGRIDPANDNTLYAEKFQ
GRVTITADTSTDTAYMELSSLRSEDTAVYYCGRGYGYYVFDHWGQGT
SEQ ID NO: 50
Humanized CD8 Light chain
CDRs are highlighted
GACATTGTGCTGACCCAGTCTCCAGCCTCCTTGGCCGTGTCTCCAGGACAGAGGGCCACCATCAC
CTGCAGAGCCAGCAAAAGTGTCAGTACATCTGGCTATAGTTATATTCACTGGTATCAGCAGAAAC
CAGGACAACCTCCTAAACTCCTGATTTACCTTGTATCCAATAAAGACACTGGGGTCCCAGCCAGG
TTCAGCGGCAGTGGGTCTGGGACCGATTTCACCCTCACAATTAATCCTGTGGAAGCTAATGATAC
TGCTAAATTATTAGCGTCAGCACATTATGGGAGCTTACACGTCGG
SEQ ID NO: 51
Humanized CD8 Light chain
CDRs are highlighted
DIVLTQSPASLAVSPGQRATITCRASKSVSTSGYSYIHWYQQKPGQPPKLLIYLVSNKDTGVPAR
FSGSGSGTDFTLTINPVEANDTAKLLAVSTLWELTRR
SEQ ID NO: 52
CDR sequence
GYSFTGYY
SEQ ID NO: 53
CDR sequence
VNPNSGGT
SEQ ID NO: 54
CDR sequence
ARSKGNYFYAMDY
SEQ ID NO: 55
CDR sequence
ASESVDSHGTSF
SEQ ID NO: 56
CDR sequence
RASN
SEQ ID NO: 57
CDR sequence
QQSNEDPPT

What is claimed is:

1. An isolated HERV-K antagonist antibody that binds to human endogenous retrovirus-K (HERV-K) envelope protein, comprising:
(a) a humanized or human framework region;
(b) a heavy chain variable region (HCVR)
comprising the CDR sequences
(SEQ ID NO: 52)
GYSFTGYY,
(SEQ ID NO: 53)
VNPNSGGT,
and
(SEQ ID NO: 54)
ARSKGNYFYAMDY;
(c) a light chain variable region (LCVR)
comprising the CDR sequences
(SEQ ID NO: 56)
ASESVDSHGTSF,
(SEQ ID NO: 56)
RASN,
and
(SEQ ID NO: 57)
QQSNEDPPT
(d) binds to full-length HERV-K envelope SU domain.

2. The antibody according to claim 1, further comprising a sequence near the boundaries for CDRs selected from the group consisting of VRQAPGKGLEW (SEQ ID NO: 46). and LQMNSLRAEDTAVYYC (SEQ ID NO: 47).

3. The antibody according to claim 1, wherein the antibody is a HUM1 antibody.

4. The antibody according to claim 1, wherein the antibody is a hu6H5 antibody.

5. The antibody according to claim 1, for use in reducing tumor growth.

6. The antibody according to claim 1, for use in reducing metastasis to lung, lymph nodes, or other organs.

7. An isolated nucleic acid comprising a nucleotide sequence encoding the HCVR, the LCVR, or a combination thereof of claim 1.

8. An expression vector comprising the nucleic acid of claim 7.

9. A host cell transformed with an expression vector of claim 8.

10. A method of treating cancer in a mammal, comprising administering a therapeutically effective amount of the antibody according to claim 1 to a mammal in need thereof.

11. The method of claim 10, wherein the antibody is conjugated to a cytotoxic drug, an auristatin, or a functional peptide analog or derivate thereof via a linker.

12. The method of claim 10, wherein the cancer is selected from the group consisting of melanoma, chronic lymphocytic leukemia, breast cancer, pancreatic cancer, head and neck cancer, ovarian cancer, cervical cancer, colorectal cancer, testicular cancer, stomach cancer, kidney cancer, endometrial cancer, uterine cancer, bladder cancer, prostate cancer, esophageal cancer, liver cancer, and non-small cell lung cancer.

13. A humanized antibody for use in CAR T, CAR NK, or BiTE assays.

14. A humanized antibody for use in CAR T, CAR NK, or BiTE assays, wherein the assays are used to develop CAR T, CAR NK, or BiTEs.

15. An isolated antibody that binds to human CD3 T cells or CD8 T cells, comprising
(a) a heavy chain variable region (HCVR), comprising the CDR sequences from SEQ ID NO. 21, SEQ ID NO. 23, SEQ ID NO. 25, or SEQ ID NO. 27; and
(b) a light chain variable region (LCVR), comprising the CDR sequences from SEQ ID NO. 29, SEQ ID NO. 31, SEQ ID NO. 32, or SEQ ID NO. 33.

16. A BiTE directed against T cell CD3 or CD8 and a humanized scFv against the tumor-associated antigen HERV-K, comprising antibodies targeting either CD3 or CD8 and HERV-K.

17. T cells expressing a lentiviral CAR expression vector that bears a humanized or fully human HERV-K scFv.

18. A humanized single chain variable fragment (scFv) antibody able to bind antigens produced from recombinant HERV-K Env surface fusion protein (KSU) and lysates from cancer cells that express HERV-K Env proteins.

19. A CAR produced from the humanized scFv of claim 18.

20. A method of blockading of the immunosuppressive domain (ISD) with immune checkpoint inhibitors of HERV-K.

21. The method of claim 20, wherein the immune checkpoint inhibitors of HERV-K are selected from the group consisting of monoclonal antibodies and drugs targeting the ISD of HERV-K.
